(19) United States
(12) Patent Application Publication
(10) Pub. No.: US 2023/0184785 A1
(43) Pub. Date: Jun. 15, 2023

(54) DEVICES, ASSAYS AND METHODS OF TESTING PREECLAMPSIA

(71) Applicant: CEDARS-SINAI MEDICAL CENTER, Los Angeles, CA (US)

(72) Inventors: S. Ananth Karumanchi, Los Angeles, CA (US); Anders H. Berg, Culver City, CA (US); Ravi Thadhani, Boston, MA (US)

(73) Assignee: CEDARS-SINAI MEDICAL CENTER, Los Angeles, CA (US)

(21) Appl. No.: 17/924,010

(22) PCT Filed: May 18, 2021
(86) PCT No.: PCT/US2021/033012

§ 371 Date: Nov. 8, 2022

Related U.S. Application Data
(60) Provisional application No. 63/026,549, filed on May 18, 2020.

(51) Int. Cl. G01N 33/68; G01N 33/543

(52) U.S. Cl. CPC G01N 33/689; G01N 33/54388; G01N 2333/4704; G01N 2333/71; G01N 2333/912; G01N 2800/368

(57) ABSTRACT

Devices based on semi-quantitative “sandwich” lateral flow immunoassay and methods of using the devices are provided to determine the presence and estimate the quantity of Flt-1 protein found in the plasma, serum, whole blood, saliva, urine or another bodily fluid of pregnant women in order to predict or screen for the risk of preeclampsia in pregnant women. Assays based on the devices are also provided.

### CROSS-REFERENCE TO RELATED APPLICATIONS

This application includes a claim of priority under 35 U.S.C. § 119(e) to U.S. provisional patent application No. 63/026,549, filed May 18, 2020, the entirety of which is hereby incorporated by reference.

### FIELD OF INVENTION

This invention relates to immunochromatography or lateral flow devices and assays (e.g., point-of care devices and assays) to detect markers indicative of preeclampsia in pregnant women as a point-of-care tool or a lab based tool.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Preeclampsia is a condition that pregnant women develop. It is marked by high blood pressure in women who have not had high blood pressure before. Preeclamptic women will have a high level of protein in their urine and often also have swelling in the feet, legs, and hands. This condition usually appears late in pregnancy, though it can happen earlier and may even develop just after delivery.

If undiagnosed, preeclampsia can lead to eclampsia, a serious condition that can put a woman and her baby at risk, and in rare cases, cause death. Women with preeclampsia who have seizures are considered to have eclampsia. Even after giving birth, women with preeclampsia may still perceive signs and symptoms of preeclampsia that lasts for 1 to 6 weeks and sometimes even longer.

Signs and symptoms of preeclampsia include swelling, protein in the urine, and high blood pressure, as well as one or more of rapid weight gain caused by a significant increase in bodily fluid, abdominal pain, severe headaches, change in reflexes, reduced urine or no urine output, dizziness, excessive vomiting and nausea, and vision changes.

Current standards to diagnose preeclampsia include having high blood pressure and one or more of the following complications after the 20^{th} week of pregnancy: proteinuria, a low platelet count, impaired liver function, signs of kidney problems other than protein in the urine, fluid in the lungs (pulmonary edema), and new-onset headaches or visual disturbances. A blood pressure reading in excess of 140/90 mm Hg is abnormal in pregnancy. As such, tests at the hospitals or clinics are often necessary such as blood test, urine analysis, fetal ultrasound or biophysical profile test.

A simple, quick and reliable testing, for example, a point-of-care test, that can be performed at the time and place of patient care, either in the clinics or by the pregnant women at home, would provide convenience to the pregnant women and the medical professional to test and monitor any sign of preeclampsia or its related disorders like eclampsia, idiopathic fetal growth restriction, or hemolysis, elevated liver enzymes, low platelet count (HELLP) syndrome.

Therefore, it is an objective of the present invention to provide devices that can collect and detect markers of preeclampsia relatively simply and suitable for use by user at home or at the clinic.

It is another objective of the present invention to provide methods of manufacturing and using the devices.

### SUMMARY

The following embodiments and aspects thereof are described and illustrated in conjunction with compositions and methods which are meant to be exemplary and illustrative, not limiting in scope.

Integrated devices for collection and measurement of soluble fms-like tyrosine kinase 1 proteins (sFlt-1) and/or bound Flt-1 proteins (e.g., bound to its ligands, vascular endothelial growth factor, VEGF, or placental growth factor, PlGF) in a biological sample of a subject are provided, which can be used by medical professionals at the point-of-care or the subject that the biological sample is obtained from. Alternatively, the devices can be sent to a lab for processing. Various embodiments herein provide that the device is a dipstick or lateral flow immunoassay device for use in screening women with suspicion or symptoms of preeclampsia.

Various embodiments of lateral flow assay devices are provided for collection and detection of the presence or absence of a measurable level of soluble fms-like tyrosine kinase 1 (sFlt-1) or bound Flt-1 proteins, as the analyte, in a sample. The lateral flow assay devices in various embodiments can contain a sample receiving region, a development region, and an indication region, each prepared from a porous material and can be in capillary contact with each other permitting a sample fluid to wick; where the indication region can have at a first location a first capture reagent or a modification capable of immobilizing the first capture reagent, and the first capture reagent can be capable of forming a complex binding another epitope of the analyte. The lateral flow assay device can further contain a first detection reagent that has a detectable label and an antibody targeting sFlt-1, the first detection reagent can be capable of forming a complex binding an epitope of the analyte and being transported from the development region to the indication region.

Various embodiments provide lateral flow devices for detection of an analyte that includes one or more circulating fms-like tyrosine kinase 1 (Flt-1) protein isoforms in a biological sample, and the lateral flow device comprising (1) a substrate, which can include or define a sample receiving region, a development region, an indication region, and a quality control region, wherein the substrate can be or at least can be composed of a porous material and each region can be in capillary contact with at least one other region to permit a sample fluid to wick from the sample receiving region to the indication region; (2) a first capture reagent or a modification capable of immobilizing the first capture reagent at a first location of the indication region; and (3) a first detection reagent that can include a detectable label modified (e.g., attached) to an anti-Flt-1 antibody. In various aspects, the first capture reagent can be capable of forming a complex binding a first epitope of the Flt-1, and the first detection reagent can be capable of forming a complex binding a second epitope of the Flt-1 (e.g., the second epitope is different from the first epitope) and being transported from the development region to the indication region.

In some embodiments of the lateral flow devices, the indication region additionally can include a second capture reagent or a modification capable of immobilizing the first capture reagent at a second location of the indication region.

In further embodiments, a lateral flow device can additionally comprise (4) a second capture reagent comprising an antibody capable of binding the first detection reagent, wherein the first detection reagent can be capable of being transported from the development region to the quality control region, and the second capture reagent can bind to a non-epitope binding domain of the first capture reagent and is not cross-reactive with the analyte.

In some embodiments of the lateral flow devices, the first detection reagent, the analyte, and the first capture reagent can form a complex and give off a signal to indicate the presence and/or level of the analyte in the biological sample based on the presence and/or intensity of the detectable label.

In some embodiments of the lateral flow devices, wherein the first detection reagent, the analyte, and the first capture reagent form a first complex and give off a first signal at the first location in the indication region, and the first detection reagent and the second capture reagent can form a second complex and give off a second signal at the quality control region, and wherein the presence of both the first signal at the first location in the indication region and the second signal at the quality control indicates the presence of the analyte in the sample.

In further embodiments, the lateral flow (assay) devices can also comprise a second capture reagent capable of binding to a house-keeping molecule in the sample, optionally a second detection reagent for the house-keeping molecule, wherein the first detection reagent, the analyte, and the first capture reagent can form a first complex and give off a first signal at the first location in the indication region, and the house-keeping molecule and the second capture reagent can form a second complex and give off a second signal at the second location in the indication region, and wherein the presence of both the first signal at the first location and the second signal at the second location can indicate the presence of the analyte in the sample and that the assay has fidelity.

Generally, the sample receiving region when contacted with the biological sample takes up the biological sample and permits release of the biological sample towards the indication region. For example, a biological sample is obtained from an oral mucosa, and so the sample receiving region when contacted with the oral mucosa takes up oral fluid and is saturated with the oral fluid to permit release of the oral fluid towards the indication region.

Some embodiments of the lateral flow devices can further contain an end flow region, which is or is at least composed of a porous material which conducts flow of the biological sample in the lateral flow device.

In various embodiments, the analyte can be any and all isoforms of soluble Flt-1, membrane-bound Flt-1, or a combination of both. In various embodiments, the first mobile detection reagent can be a monoclonal or polyclonal antibody immunoreactive with the Flt-1. In various embodiments, the first detectable label can be selected from a group consisting of a colloidal metal, colored particles, a liposome filled with a colored substance, an enzyme, a radiolabel, a chromophore and a fluorophore.

In some embodiments, the lateral flow devices can further comprise a housing having a cavity and an inspection site on the housing, wherein the indication region extends into the cavity along the housing to the inspection site to enable visual inspection of the first location and/or the second location of the indication region. In various embodiments, visual inspection can be read by a human. In various embodiments, visual inspection can be read by a machine.

In some embodiments, an Ahlstrom Grade 1281 pad can be the sample receiving component of the lateral flow devices and can be positioned in/as the sample receiving region; an Ahlstrom Grad 6614 pad can be a conjugate pad of the lateral flow devices and can be positioned in/as the development region; a nitrocellulose CN95 membrane is (as least part of) can be an indication component of the lateral flow devices and can be positioned in/as the indication region; and/or an Ahlstrom Grade 243 pad can be an end flow component of the lateral flow devices and can be positioned in/as the end flow region.

In some aspects, the sample receiving component (e.g., an Ahlstrom Grade 1281 pad) and/or the indication component (e.g., an Ahlstrom Grade 1281 pad) can contain a detergent and magnesium chloride to reduce nonspecific binding. For example, the sample receiving component and/or the indication pad component (e.g., Ahlstrom Grade 1281 pad) can be presaturated with 0.5% Pluronic F127 detergent and 1M magnesium chloride as matrix reagents to block nonspecific binding within the development region and/or the indication region.

In various embodiments of the lateral flow devices, the sample receiving region, the development region and the indication region can be in the form of a strip positioned above a base. In further embodiments, the lateral flow devices are configured for collecting the biological sample, wherein the biological sample is plasma, serum, whole blood, saliva, and/or urine from a pregnant woman.

Assays are provided for diagnosing the presence or absence of risk of preeclampsia in a pregnant woman, which can include contacting a biological sample (e.g., saliva, plasma or whole blood, or urine) of the pregnant woman with the sample receiving region of the devices, and diagnosing the presence of risk of preeclampsia when a positive indication of the presence of sFlt-1 isoforms on the devices is observed, or diagnosing the absence of risk of preeclampsia when no positive indication of the presence of sFlt-1 on the devices is observed. In further aspects, a pregnant woman diagnosed with the absence of risk of preeclampsia is monitored at home or in a clinic; and a pregnant woman diagnosed with the presence of risk of preeclampsia is referred to emergency room or hospital for further medical evaluation.

Various embodiments provide methods of assaying a biological sample, or detecting a level, or a presence or absence, of an analyte in the biological sample, the analyte comprising soluble fms-like tyrosine kinase 1 (sFlt-1), bound Flt-1, or both, with a lateral flow device described herein. The methods can include applying the biological sample to the sample receiving region of the lateral flow device, so as to permit the biological sample to flow to the indication region, and detecting the level, or the presence or absence, of the first detectable label at the first location in the indication region. Generally, in the presence of the analyte, the first detection reagent, the analyte, and the first capture reagent form a complex.

In some embodiments of the methods of assaying a biological sample, or detecting a level, or a presence or absence, of the analyte in the biological sample, the first detectable label can be at a detectable level within 15 minutes from the application of the biological sample.

In various embodiments, the biological sample is plasma, serum or whole blood from a pregnant woman. In various embodiments, the sFlt-1 or bound Flt-1 can comprise an isoform encoded by mRNA of sFlt-1-il3-short, sFlt1-il3-long, sFlt1-il4, sFlt1-e15a, sFlt1-e15b, or mFlt-1.

In some embodiments, the analyte comprises sFlt-1, and the biological sample can be obtained from a pregnant woman 18 weeks or later of pregnancy or until 20 weeks postpartum.

Further embodiments of the methods of assaying a biological sample, or detecting a level, or a presence or absence, of the analyte in the biological sample, can further include selecting a pregnant woman 18 weeks or later of pregnancy, before applying the biological sample obtained from the pregnant woman to the lateral flow device, wherein the analyte comprises sFlt-1.

Various embodiments provide methods of assaying a biological fluid sample of a pregnant woman, or determining the presence or absence of circulating fms-like tyrosine kinase 1 (Flt-1) protein isoforms in the biological fluid sample, with a lateral flow device described herein, and the methods can include applying the biological fluid sample to the sample receiving region of the lateral flow device, whose first immobile capture reagent is a monoclonal or polyclonal antibody only immunoreactive to Flt-1, so as to permit the biological fluid sample to flow to the indication region, and detecting the presence or absence of the detectable label of the first detection reagent at the first location in the indication region, wherein, in the presence of Flt-1, the first detection reagent, the analyte, and the first capture reagent can form a complex that can be deposited at the first location in the indication region, and wherein the first detection reagent and the second capture reagent form another complex that can be deposited at the quality control region.

In some aspects, the methods can further include detecting an amount of the detectable label of the first detection reagent at the first location in the indication region to indicate a quantity of the Flt-1 protein fragments in the biological fluid sample.

In some aspects, sFlt-1, including one or more of its isoforms or mRNA splicing variants, or its fragments can be detected with the lateral flow devices, or in the methods disclosed herein, in a biological sample of plasma, serum, or both; whereas Flt-1, sFlt-1 (including one or more isoforms), or both, can be detected with the lateral flow devices, or in the methods disclosed herein, in a biological sample of whole blood.

Various embodiments of the invention provide for methods of determining a predisposition to preeclampsia or its related disorder, diagnosing preeclampsia or its related disorder, determining the likelihood of recurrence of preeclampsia or its related disorder, providing a prognosis for a subject with preeclampsia or its related disorder in a subject, or selecting a subject with preeclampsia or its related disorder for treatment with a therapy. These methods generally include contacting a sample obtained from the subject with the sample receiving region of a lateral flow device described herein, and detecting an amount of fms-like tyrosine kinase 1 (Flt-1) in the sample above a reference level. In some aspects, the reference level can be an amount of sFlt-1 from a sample of a non-pregnant woman or a woman not having preeclampsia, or an average amount of sFlt-1 from samples of a group of non-pregnant women or women not having preeclampsia. The size of the group can be for example, about 25, 50, 100, 200, 250, 500. In some aspects, the reference level can be a cut-off value discussed herein.

Exemplary preeclampsia or its related disorders can include preeclampsia or eclampsia with severe features, wherein the severe features comprises severe hypertension, or hypertension with one or more of thrombocytopenia, renal insufficiency, cerebral or visual symptoms, impaired liver function, and pulmonary edema; or an adverse outcome related to preeclampsia, wherein the adverse outcome related to preeclampsia comprises elevated liver function test, low platelet count, placental abruption, pulmonary edema, cerebral hemorrhage, convulsion, acute renal insufficiency, or maternal death; or the preeclampsia-related disorder comprises eclampsia, idiopathic fetal growth restriction, or hemolysis, elevated liver enzymes, low platelet count (HELLP) syndrome.

In various embodiments of the detection methods or processes described herein, the methods or processes can further include digitally measuring an amount of the first signal and the amount of the second signal, such as operating an application on a mobile or portable device. In other embodiments, the methods or processes can further include operating a densitometry instrument to obtain the amount of the Flt-1 protein fragments.

Various embodiments of the invention provide for methods of administering a therapy for treatment and/or management of preeclampsia or eclampsia to a patient in need thereof, or selecting a patient for the therapy, and the methods include detecting an amount of fms-like tyrosine kinase 1 (Flt-1) protein fragments using a lateral flow device described herein, and administering an effective amount of a steroid, magnesium sulfate, an anti-Flt-1 antibody and/or therapeutic apheresis to lower the sFlt1 in the patient in need thereof for treating and/or reducing the progression of preeclampsia or eclampsia.

Methods of manufacturing the lateral flow assay devices, as well as using the devices are also provided. In various embodiments, methods of manufacturing a lateral flow device for detecting circulating fms-like tyrosine kinase 1 (Flt-1) protein fragment include (1) providing a base and (2) providing a substrate positioned above the base, the substrate defining: a sample receiving region, an indication region, and optionally a development region positioned between the sample receiving region and the indication region, or said optional development region overlapping with the sample receiving region and/or overlapping with the indication region, wherein each region comprises a porous material and is in capillary contact with at least one other region, thereby permitting a fluid to wick from the sample receiving region to the indication region; (3) immobilizing a first capture reagent or a modification capable of binding the first capture reagent at a first location in the indication region, wherein the first capture reagent comprises a monoclonal or polyclonal antibody specifically immunoreactive with Flt-1, or an antigen-binding fragment thereof; (4) providing a first detection reagent comprising a detectable label and an antibody or fragment thereof capable of binding the Flt-1, wherein the first detection reagent is capable of being transported from the development region to the indication region, whereby the sample receiving region is configured to receive a fluid sample containing one or more analytes and to permit the fluid sample to wick to the indication region, and when the analytes comprise Flt-1, a complex is formed comprising the first detection reagent, the Flt-1, and the first capture reagent, and the complex indicates the presence of the Flt-1 through the detectable label at the first location in the indication region.

In some embodiments, the methods of manufacturing a lateral flow device can further include (5) providing at a second location in the indication region a second capture reagent or a modification capable of immobilizing the second capture reagent, (6) providing a second detection reagent capable of being transported to the indication region, the second detection reagent is capable of binding a house-keeping molecule in the fluid sample, and the second detection reagent, the house-keeping molecule and the second capture reagent form a complex to indicate a presence of the house-keeping molecule through the second detection reagent, wherein the second mobile detection reagent is not cross-reactive with Flt-1, with the first detection reagent or with the first capture reagent, and optionally (7) further providing an end flow region comprising a porous material and positioned such that a fluid is conducted from the sample receiving region through the indication region.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1A is a diagram showing a lateral flow device of an embodiment of the invention. Components are not drawn to scale.

FIG. 1B is an image showing the result of half strip test of DuoSet capture latex conjugate. FIG. 1C is an image showing the result of half strip test of Clone 49560 latex conjugate.

FIG. 1D is an image showing the result of half strip test of Clone 611926 latex conjugate. FIG. 1E is an image showing the result of strip test of biotin-avidin dose response. FIG. 1F is an image showing the result of half strip test of Clone 611926 CNB and gold conjugates with buffer. FIG. 1G is an image showing the result of full strip test of salt with 40:1 latex conjugate and ‘good’ plasma. FIG. 1H is an image showing the result of full strip test comparing polystreptavidin and streptavidin at 30-minute development time. FIG. 1I is an image showing the result of full strip test of saliva sample screen with polystreptavidin test line. FIG. 1J is an image showing the result of full strip test of spiking saliva with high sFlt-1 level plasma. FIG. 1K is an image showing the result of full strip test of client sample panel 6.

FIGS. 2-9 are top-down images of 92 plasma samples, each run on an immunochromatographic assay device in the shape of a strip, where samples were wicked to laterally flow in the indicated direction by the arrow. The coloration at the top (downstream) of each device in each image, indicated by a line (“—”), shows a control of the assay. The coloration that is upstream of the control in each device, indicated by a double line (“=”), shows an indication region where the analyte, sFlt-1, in a complex with a colored detection reagent is immobilized by a capture reagent specific to the analyte. Known concentrations of sFlt-1 in each sample and name of each sample are annotated above each device. Visual grade (“VG”) of the coloration (“=”) at the indication region is determined and designated below the devices, where a more intense coloration is designated with a larger integer as its VG. FIG. 2 depicts plasma samples 1-12. FIG. 3 depicts plasma samples 13-24. FIG. 4 depicts plasma samples 25-36. FIG. 5 depicts plasma samples 37-48. FIG. 6 depicts plasma samples 49-60. FIG. 7 depicts plasma samples 61-72. FIG. 8 depicts plasma samples 73-84. FIG. 9 depicts plasma samples 85-92.

FIG. 10A is a chart plotting the visual grade information of each of the 92 samples in an ascending order based on their known concentrations of sFlt-1.

FIG. 10B is a chart plotting visual grade information of 89 of the 92 samples in an ascending order based on their known concentrations of sFlt-1. Three samples denoted 061 d3, 061 wk2, and 047 d0 are not plotted.

FIG. 11 is an image of the visual grade scale used to assign a visual intensity score to test lines and control lines. A visual grade of ≥2 is considered visible by a user and a visual grade of ≤1 is considered not visible by a user.

FIG. 12 depicts exemplary embodiments of cassette designs with double-sample port or single-sample port.

FIGS. 13A-13C are images of the visual grade scale used to assign a visual intensity score for saliva samples tested in the dipstick immunoassay devices. FIGS. 13A and 13B show the imaging results from full strips run with 15 μL of spiked and unspiked saliva samples that were developed for 15 minutes and 30 minutes, respectively. FIG. 13C shows the imaging results from full strips run with 15 μL of spiked and unspiked saliva samples.

FIG. 14A shows results from full strips run with 15 μL of saliva sample 4.

FIG. 14B shows results from full strips run with 15 μL of saliva samples 4 and 6 that were thawed after frozen.

FIG. 14C shows results from full strips run with 15 μL of saliva samples 9 and 10 that were thawed after frozen. This experiment tested interference by freezing and thawing and centrifuging samples.

FIG. 15A results from full strips run with saliva samples on both dipstick and lateral flow immunoassay devices. This experiment also optimized volume of saliva added.

FIG. 15B shows results from full strips run with 15 μL of spiked and unspiked saliva samples.

FIG. 16A shows results from full strips run with 15 μL of saliva samples that were pre-diluted in chase buffer.

FIG. 16B shows results from full strips run with 15 μL of saliva samples and where the polystreptavidin capture reagent concentration was lowered.

FIG. 17 show a generic cassette with an assay of sFlt-1 in Example 11.

FIG. 18 is a chart showing visual grade result against sFlt-1 concentration.

FIG. 19 is a chart showing visual grade data compared to their known concentration of sFlt-1 from testing the 92 characterized samples in house.

FIG. 20 is a chart showing visual grade data compared to their known concentration of sFlt-1 from testing the 295 characterized samples in house.

FIG. 21A is a measurement using Axxin Densitometry peak values of plasma sFLT1 of known concentrations on the lateral flow device. FIG. 21B is a visual grade result measuring plasma sFLT1 of known concentrations on the lateral flow device.

FIGS. 22A and 22B exemplify that negative LFA test results (defined by peak values less than 1000 units by Axxin Densitometer) have 97% negative predictive value (NPV) for prediction of PE with severe features (sPE) within 2 weeks. FIG. 22A depicts a result overview of 190 patient's samples measured using a LFA device, and whether they developed or did not develop sPE in the subsequent two weeks. (*Two-week outcome consistent with PE with severe features (sPE)). FIG. 22B is a graph showing the changes of negative predictive value (NPV) and positive predictive value (PPV) at varying LFA values; and that at a cut-point of 1000, Gl PE test has a 97% NPV.

FIG. 23A is a top-down view in a picture of a lateral flow device (strip), with a sample pad (as a sample receiving region), a conjugate pad (as a development region), the membrane (as an indication region), and a wick pad (as an end flow region). This corresponds to the side view shown in FIG. 1A.

FIG. 23B is a picture of a strip placed into a borosilicate glass tube for a test, as described in Example 20.

FIG. 23C shows images of strips placed in the holder for insertion into the drawer of an Axxin Densitometer, as described in Example 20.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., March, Advanced Organic Chemistry Reactions, Mechanisms and Structure 7^{th} ed., J. Wiley & Sons (New York, N.Y. 2013); and Sambrook and Russel, Molecular Cloning: A Laboratory Manual 4^{th} ed., Cold Spring Harbor Laboratory Press (Cold Spring Harbor, N.Y. 2012), provide one skilled in the art with a general guide to many of the terms used in the present application. For references on how to prepare antibodies, see D. Lane, Antibodies: A Laboratory Manual 2^{nd} ed. (Cold Spring Harbor Press, Cold Spring Harbor N.Y., 2013); Kohler and Milstein, (1976) Eur. J. Immunol. 6: 511; Queen et al. U.S. Pat. No. 5,585,089; and Riechmann et al., Nature 332: 323 (1988); U.S. Pat. No. 4,946,778; Bird, Science 242:423-42 (1988); Huston et al., Proc. Natl. Acad. Sci. USA 85:5879-5883 (1988); Ward et al., Nature 334:544-54 (1989); Tomlinson I. and Holliger P. (2000) Methods Enzymol, 326, 461-479; Holliger P. (2005) Nat. Biotechnol. September; 23(9):1126-36).

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

A “subject” means a human or an animal. In various embodiment, the subject is a woman (human). In various embodiments, the subject is a pregnant human. In further aspects, the subject is a pregnant woman. In yet another aspect, the subject is a woman having recently given birth, such as in the past month or months. In some embodiments, the subject is a post-partum human. In some embodiments, the subject is a pregnant human or a post-partum human. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, mouse, rat, dog, cat, horse, or cow, but are not limited to these examples.

A “patient in need of” or “subject in need” of treatment for a particular disease, disorder, or condition may be a subject suspected of having that disease, disorder, or condition, diagnosed as having that disease, disorder, or condition, already treated or being treated for that disease, disorder, or condition, not treated for that disease, disorder, or condition, or at risk of developing that disease, disorder, or condition. In various embodiments, a patient in need thereof is a human patient.

“Sample” or “biological sample” in various embodiments refers to bodily fluid, tissue or a specimen obtained from a subject. Exemplary biological samples used in the present invention (e.g., for the sFlt-1 assay in the lateral flow devices (or also referred to as immunochromatographic devices)) include but are not limited to plasma, serum, whole blood, saliva, urine, and mucus. In various embodiments, the sample is plasma. In various embodiments, the sample is whole blood. In various embodiments, saliva samples are clarified saliva. Clarified saliva refers to whole saliva that has been filtered through a swab or other form of mechanical filtration, or that has been frozen and/or centrifuged to pelletize mucins. In various embodiments, the biological sample for use with the devices is a plasma. Biological samples may be freshly obtained, or frozen and then thawed before testing. In various embodiments, the devices utilize freshly obtained plasma (without refrigeration or freezing, and within 6 hours, 12 hours or 1 day after isolation from a mammalian body; or methods of using the devices include contacting freshly obtained plasma with the devices, generally the sample receiving region of the devices. In other embodiments, the devices utilize freshly obtained saliva; and methods of using the devices include contacting freshly obtained saliva with the devices, e.g., the sample receiving region of the devices. In certain embodiments, the blood or a component thereof is plasma and one or more methods described herein comprises removing a volume of the subject's blood and separating the blood into plasma and cellular components.

The term “antibody” refers to an intact immunoglobulin or to a monoclonal or polyclonal antigen-binding fragment with the Fc (crystallizable fragment) region or FcRn binding fragment of the Fc region, referred to herein as the “Fc fragment” or “Fc domain”. Antigen-binding fragments may be produced by recombinant DNA techniques or by enzymatic or chemical cleavage of intact antibodies. Antigen-binding fragments include, inter alia, Fab, Fab′, F(ab′)2, Fv, dAb, and complementarity determining region (CDR) fragments, single-chain antibodies (scFv), single domain antibodies, chimeric antibodies, diabodies and polypeptides that contain at least a portion of an immunoglobulin that is sufficient to confer specific antigen binding to the polypeptide. The Fc domain includes portions of two heavy chains contributing to two or three classes of the antibody. The Fc domain may be produced by recombinant DNA techniques or by enzymatic (e.g., papain cleavage) or via chemical cleavage of intact antibodies. An antibody can be a chimeric, humanized or human antibody. An antibody can be an IgG1, IgG2, IgG3 or IgG4 antibody. In some aspects, an antibody herein has an Fc region that has been modified to alter at least one of effector function, half-life, proteolysis, or glycosylation.

The term “antibody fragment,” refers to a protein fragment that comprises only a portion of an intact antibody, generally including an antigen binding site of the intact antibody and thus retaining the ability to bind antigen. Examples of antibody fragments encompassed by the present definition include: (i) the Fab fragment, having V_{L}, C_{L}, V_{H} and CH1 domains; (ii) the Fab′ fragment, which is a Fab fragment having one or more cysteine residues at the C-terminus of the CH1 domain; (iii) the Fd fragment having V_{H} and CH1 domains; (iv) the Fd′ fragment having V_{H} and CH1 domains and one or more cysteine residues at the C-terminus of the CH1 domain; (v) the Fv fragment having the V_{L} and V_{H} domains of a single arm of an antibody; (vi) the dAb fragment which consists of a V_{H} domain; (vii) isolated CDR regions; (viii) F(ab′)2 fragments, a bivalent fragment including two Fab′ fragments linked by a disulphide bridge at the hinge region; (ix) single chain antibody molecules (e.g., single chain Fv; scFv); (x) “diabodie” with two antigen binding sites, comprising a heavy chain variable domain (V_{H}) connected to a light chain variable domain (V_{L}) in the same polypeptide chain; (xi) “linear antibodies” comprising a pair of tandem Fd segments (V_{H}-CH1-V_{H}-CH1) which, together with complementary light chain polypeptides, form a pair of antigen binding regions. An antibody or antibody fragment can be scFvs, camelbodies, nanobodies, IgNAR (single-chain antibodies derived from sharks) and Fab, Fab′ or F(ab′)₂ fragment.

“Specifically immunoreactive,” “selectively immunoreactive,” “selectively binds” or “specifically binds” in various embodiments refers to the ability of an antibody or antibody fragment thereof described herein to bind to a target, such as an analyte in the biological sample, with a K_{D} 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. In some embodiments, “specifically immunoreactive” to one antigen or a target molecule of an antibody or antibody fragment indicates that the antibody or antibody fragment does not bind or binds to a non-antigen at a level that is at least two-, three-, or four-order of magnitude lower compared to when it binds to the intended antigen or target molecule. Specific binding can be influenced by, for example, the affinity and avidity of the polypeptide agent and the concentration of polypeptide agent. The person of ordinary skill in the art can determine appropriate conditions under which the polypeptide agents described herein selectively bind the targets using any suitable methods, such as titration of a polypeptide agent in a suitable cell binding assay.

Membrane materials generally contain porous structures. In some embodiments, the porous structure of the membrane is large/void enough to allow transport of sample fluid or test buffers, as well as the migration of analyte, antibodies (including detectable label-modified antibodies), and bound complexes between analyte and antibodies, at least driven by a capillary force. In some embodiments, the porous structure of the membrane has layers where at least the exterior layer(s) are “tight” enough to trap or enclose the analyte, antibodies (including detectable label-modified antibodies) and bound complexes within the membrane. Membrane materials and/or structures are selected to afford desirable speed to result, assay sensitivity, reproducibility at the threshold (cut-off value for the analyte) and in a dynamic range.

Elevated concentrations of soluble fms-like tyrosine kinase 1 (sFlt-1) or membrane-bound Flt-1 proteins (mFlt-1) in salivary, plasma or whole blood, urine, or another blood fluid are associated with an increased risk or presence of preeclampsia in pregnant mothers. One or more isoforms of Flt-1 (e.g., Flt-1 proteins produced from different mRNA isoforms) can be measured in the devices or methods described in the present invention, including but are not limited to those encoded by mRNAs of sFlt-1-il3-short, sFlt1-il3-long, sFlt1-il4, sFlt1-e15a, sFlt1-e15b and/or mFlt-1. Further descriptions of Flt-1 isoforms are provided in Placenta, volume 30, issue 3, pages 250-255, March 2009, and in Scientific Reports, volume 7, Article number: 12139 (2017), which is hereby incorporated by reference. For example, Genbank accession number AF063657 provides a nucleotide (mRNA) and amino acid sequences of human Flt-1. Additional examples of sFlt-1 mRNA splice variants are seen in Genbank accession numbers U01134, BC039007, AI188382, N47911, AA035437, BF061039, BG435852; and Flt1 (previous nomenclature: VEGFR-1) is seen in Genbank accession number NM002019. sFlt-1 is a soluble form of Flt-1, which lacks the transmembrane and cytoplasmic domains of the full-length Flt-1 receptor. sFlt-1 binds to VEGF with high affinity but does not stimulate mitogenesis of endothelial cells. Without wishing to be bound by a particular theory, sFlt-1 acts as a “physiologic sink” to bind to and deplete the trophoblast cells and maternal endothelial cells of functional growth factors required for the proper development and angiogenesis of the fetus and/or the placenta. In some aspects, a lateral flow device described herein is configured for detecting sFlt-1, including one or more of its isoforms or mRNA splicing variants, or its fragments in a biological sample of plasma, serum, or both. In some aspects, a lateral flow device described herein is not configured for detecting full-length Flt-1 in a biological sample of plasma or serum. In some aspects, a lateral flow device described herein is configured for detecting Flt-1, sFlt-1 (including one or more isoforms), or both, in a biological sample of whole blood.

In some embodiments, symptoms of pre-eclampsia include any one or more of the following: (1) a systolic blood pressure (BP)>140 mmHg and a diastolic BP>90 mmHg after 20 weeks gestation, (2) new onset proteinuria (1+ by dipstik on urinanalysis, >300 mg of protein in a 24 hour urine collection, or random urine protein/creatinine ratio >0.3), or (3) non-resolution of hypertension and proteinuria by 12 weeks postpartum. In some embodiments, the symptoms of pre-eclampsia include 2 or more of the 3 aforementioned parameters. In some embodiments, the symptoms of pre-eclampsia include all 3 of the aforementioned parameters. In some embodiments, the symptoms of pre-eclampsia include renal dysfunction and glomerular endotheliosis or hypertrophy. In some embodiments, symptoms of eclampsia further include any of the following symptoms due to pregnancy or the influence of a recent pregnancy: seizures, coma, thrombocytopenia, liver edema, pulmonary edema, or cerebral edema.

In some embodiments, devices disclosed herein are used to identify preeclampsia with severe features (defined as preeclampsia with severe hypertension (≥160 mmHg systolic or ≥110 mmHg diastolic) or hypertension with any of the following features: thrombocytopenia (<100,000 platelets/mL), renal insufficiency (serum creatinine concentrations >1.2 mg/dL), cerebral or visual symptoms, impaired liver function (elevated blood concentrations of liver transaminases, ALT or AST ≥80 (U/L) or pulmonary edema).

In some embodiments, devices disclosed herein are used to identify adverse outcomes related to preeclampsia. Adverse maternal outcomes included: elevated liver function tests (aspartate aminotransferase (AST) or alanine aminotransferase (ALT) (≥80 U/L)), low platelet count (≤100K/uL), placental abruption (clinical or pathological diagnosis), pulmonary edema, cerebral hemorrhage, convulsion (in the absence of a preexisting seizure disorder), acute renal insufficiency (creatinine >1.1 mg/dL), or maternal death. The adverse fetal/neonatal outcomes include small for gestational age birth weight (≤10th percentile for gestational age) with or without abnormal umbilical artery Doppler (absent or reverse flow), fetal death, and neonatal death.

### Devices

Various embodiments of present invention provide integrated devices are provided for collection of a biological sample and measurement of sFlt-1 or bound Flt-1 (e.g., mFlt-1), including any of the isoforms, in the biological sample (e.g., saliva, blood, blood serum, whole blood, or urine), which can be used by medical caregivers at the point-of-care, out-patient use, in-patient use, sent to a laboratory, or by everyday consumers. In some aspects, the devices are used for collection of a biological sample. In some aspects, the devices are used for detecting a level of sFlt-1 and/or bound Flt-1, as well as any of the isoforms, in a biological sample. In some aspects, the devices are used for detecting a level of sFlt-1 in a biological sample. Various embodiments of the devices include a layer or layers of porous structured materials (e.g., membranes), which allows for transport of molecules through advection, diffusion or a capillary force by fluid, and one or more reagents entrapped or embedded in the porous structured materials, which allows for interaction with one or more target molecules from the biological sample and detection of the presence and/or quantity of the target molecules. In certain aspects, the transport of molecules is along the porous structured materials from one end to the other, the reagents include antibodies or antibody fragment to allow for specificity in the interaction with target molecules, and the detection of the presence and/or quantity of the target molecules is through a detectable label, hence the devices configured for lateral flow immunochromatographic assays.

Various embodiments provide that a device contain components (detachable to each other), regions (connected to one another or on a same piece of unbroken material), or a combination thereof, which include one or more of 1) a sample receiving component/region, 2) a development region/component, 3) an indication region/component, and 4) a wicking component/region (e.g., as an end flow or an underside).

Typically, sample receiving components/regions include a material that preferably is a hydrophilic material which facilitates absorption and transport of a fluid sample to an underlying or nearby chromatographic substrate. Cotton fibers, rayon fibers, glass fibers, or a combination thereof are exemplary materials to form a sample receiving component/region. Suitable materials to form a sample receiving component/region include cellulose acetate, hydrophilic polyester, and other materials having similar properties. Further, a combination of absorbent materials also may be used. Non-limiting examples of useful materials include bonded cellulose acetate, bonded polyolefin, or hydrophilic polyester, such as those materials commercially available from Filtrona Fibertec Company (Colonial Heights, Va.). Other useful materials include absorbent matrices, such as Grade 939, Grade 989, Grade 1278, or Grade 1281, available from Ahlstrom Corporation. One embodiment provides that a sample receiving region/component is prepared from Ahlstrom Grade 1281 for collecting of saliva or plasma or urine and transportation of sFlt-1 or bound Flt-1 in the saliva or plasma to a chromatographic substrate in the device. Sample receiving components/regions are in various aspects in the form of a pad, strip, membrane, layers of membrane, or container. Further embodiments provide the sample receiving components/regions are preferably coated or treated with a buffered solution containing a salt, a carrier protein and/or a surfactant. The presence of the salt, carrier protein and/or surfactant reduces non-specific adsorption of the analyte, e.g., Flt-1. In various embodiments, a concentration of about 0.1M, 0.2 M, 0.3M, 0.4M, 0.5M, 0.6M, 0.7M, 0.8M, 0.9M, 1M, 1.1M, 1.2M, 1.3M, 1.4M or 1.5M MgCl₂ and about 0.1%, 0.2%, 0.3%, 0.4%, 0.5%, 0.6%, 0.7%, 0.8%, 0.9%, or 1% PLURONIC F127 can be effective for this purpose. In various embodiments, a concentration of about 0.5M-1.5M MgCl₂ and about 0.5%-1.5% PLURONIC F127 can be effective for this purpose. In various embodiments, a concentration of about 0.75M-1.25M MgCl₂ and about 0.75%-1.25% PLURONIC F127 can be effective for this purpose. In various embodiments, a concentration of about 1.5M-2.0M MgCl₂ and about 1.5%-2.0% PLURONIC F127 can be effective for this purpose. In various embodiments, the following concentrations can be effective for this purpose: (1) 0.6M MgCl₂ and 1% PLURONIC F127; (2) 1.0M MgCl₂ and 1% PLURONIC F127; (3) 1.5M MgCl₂ and 1% PLURONIC F127; (4) 1.0M MgCl₂ and 0.5% PLURONIC F127; or (5) 1.0M MgCl₂ and 2% PLURONIC F127.

In various embodiments, the sample receiving components/regions are about 22 mm in length. In various embodiments, the sample receiving components/regions are about 20-25 mm in length. In various embodiments, the sample receiving components/regions are about 15-20 mm in length. In various embodiments, the sample receiving components/regions are about 25-30 mm in length. In various embodiments, the sample receiving components/regions are about 10-15 mm in length.

Typically, development regions/components include a material which facilitates transport of free antibodies or antibody fragments or a complex formed by analyte-antibody or analyte-antibody fragment to an underlying or nearby chromatographic substrate, allowing interaction or binding between analyte and antibody (or antibody fragment). Non-limiting examples of useful material for the conjugate pad include Grade 6614, Grade 8980 and Grade 8914, available from Ahlstrom Corporation. In some aspects, the development regions/components contain a conjugate pad. In some aspects, the development regions/components contain overlaying conjugate pad on top of a chromatographic substrate. In some aspects, the conjugate pad has overlapping contact in one area with the material in the sample receiving component/region. In other aspects, the development component does not form an individual region, as a development region is merged with the sample receiving region; and hence, the device may include a sample pad and a conjugate pad in a combined sample receiving (and development) region, a membrane as an indication region modified at a certain location with a capture reagent or a modification to immobilize the capture reagent, and a wick pad as an end flow region. In various embodiments, the development regions/components are about 22 mm in length. In various embodiments, the development components/regions are about 20-25 mm in length. In various embodiments, the development components/regions are about 15-20 mm in length. In various embodiments, the development components/regions are about 25-30 mm in length. In various embodiments, the development components/regions are about 10-15 mm in length. In various embodiments, the development components/regions are about 15-25 mm in length.

In some instances, the chromatographic substrate (e.g., membrane pad) can include an anti-analyte antibody-particle conjugate at least in a first location and an analyte-conjugate protein at least in a second location. One embodiment provides that a development region/component has a conjugate pad that is prepared with Ahlstrom Grade 6614 and contains one or more anti-Flt-1 antibodies that are either modified with a detectable label, or with a functional group that is capable of binding to an indication region on the chromatographic substrate of the device. A concentration of about 0.1% (or about 0.01%-1%, 0.02%-0.8% m 0.04%-0.6%, 0.06%-0.4%, 0.08%-0.2%, 0.01%-0.05%, 0.06%-0.10%, 0.11%-0.15%, 0.16%-0.20%, 0.21%-0.25%, 0.26%-0.30% 0.31%-0.35%, 0.36%-0.40%, 0.41%-0.45%, or 0.46%-0.50% w/w or v/v) colored (e.g., red) latex bead-modified anti-hVEGFR antibody with a number ratio of 40:1 (latex bead:antibody) (or between 60:1 and 20:1, between 70:1 and 10:1, or between 50:1 and 30:1; ora ratio of about 20:1, 25:1, 30:1, 35:1, 40:1, 45:1, 50:1, 55:1, or 60:1) is effective for binding the analyte, sFlt-1; alternatively or in combination, a concentration of 22.5 μg/mL anti-hVEGFR antibody (or between) is effective for this purpose. Concentrations of about 15 μg/mL, 16 μg/mL, 17 μg/mL, 18 μg/mL, 19 μg/mL, 19.5 μg/mL, 20 μg/mL, 20.25 μg/mL, 20.50 μg/mL, 20.75 μg/mL, 21 μg/mL, 21.25 μg/mL, 21.5 μg/mL, 21.75 μg/mL, 22 μg/mL, 22.25 μg/mL, 22.50 μg/mL, 22.75 μg/mL, 23 μg/mL, 23.25 μg/mL, 23.5 μg/mL, 24 μg/mL, 24.25 μg/mL, 24.5 μg/mL, 24.75 μg/mL, 25 μg/mL, 25.5 μg/mL, 26 μg/mL, 27 μg/mL, 28 μg/mL, 29 μg/mL, or 30 μg/mL anti-hVEGFR antibody. Other colored latex beads can also be used; for example, blue, orange, yellow-green, range or dark red.

In various embodiments, a concentration of about 0.1% (or about 0.01%-1%, 0.02%-0.8% m 0.04%-0.6%, 0.06%-0.4%, 0.08%-0.2%, 0.01%-0.05%, 0.06%-0.10%, 0.11%-0.15%, 0.16%-0.20%, 0.21%-0.25%, 0.26%-0.30% 0.31%-0.35%, 0.36%-0.40%, 0.41%-0.45%, or 0.46%-0.50% w/w or v/v) colored (e.g., red) latex bead-modified anti-sFLT-1 antibody and/or anti-mFLT-1 antibody with a number ratio of 40:1 (latex bead:antibody) (or between 60:1 and 20:1, between 70:1 and 10:1, or between 50:1 and 30:1; or a ratio of about 20:1, 25:1, 30:1, 35:1, 40:1, 45:1, 50:1, 55:1, or 60:1) is effective for binding the analyte, sFlt-1; alternatively or in combination, a concentration of 22.5 μg/mL anti-hVEGFR antibody (or between) is effective for this purpose. Concentrations of about 15 μg/mL, 16 μg/mL, 17 μg/mL, 18 μg/mL, 19 μg/mL, 19.5 μg/mL, 20 μg/mL, 20.25 μg/mL, 20.50 μg/mL, 20.75 μg/mL, 21 μg/mL, 21.25 μg/mL, 21.5 μg/mL, 21.75 μg/mL, 22 μg/mL, 22.25 μg/mL, 22.50 μg/mL, 22.75 μg/mL, 23 μg/mL, 23.25 μg/mL, 23.5 μg/mL, 24 μg/mL, 24.25 μg/mL, 24.5 μg/mL, 24.75 μg/mL, 25 μg/mL, 25.5 μg/mL, 26 μg/mL, 27 μg/mL, 28 μg/mL, 29 μg/mL, or 30 μg/mL anti-hVEGFR antibody. Other colored latex beads can also be used; for example, blue, orange, yellow-green, range or dark red.

In these embodiments, the sFLT-1 or mFLT-1 can be any one or more isoforms of Flt-1 or fragments thereof, including but are not limited to those encoded by mRNAs of sFlt-1-il3-short, sFlt1-il3-long, sFlt1-il4, sFlt1-e15 a, sFlt1-e15b and mFlt-1.

Indication regions/components of the devices generally refer to an area where analytes or target molecules in the biological sample are indicated of their presence and/or amounts. Various embodiments provide the presence and/or amounts of analytes are detected and/or quantified due to detectable labels that are in association with antibodies or antibody fragments that bind and are in complex with the analytes or target molecules. Exemplary indication regions/components include a chromatographic substrate, such as a chromatographic membrane (e.g., in the form of a pad). In some instances, the chromatographic substrate (e.g., membrane pad) can include an anti-analyte antibody-particle conjugate at least a first location and an analyte-conjugate protein at least a second location.

In some embodiments, the chromatographic substrate (e.g., membrane pad) further comprises an anti-IgG antibody (or another anti-immunoglobulin antibody) at a third location—as an internal quality control region—which indicates binding of plasma IgG to the quality control region, and is an indicator of successful separation and flow of plasma by capillary action across the entirety of the sample pad, conjugate pad, and membrane. Development and appearance of a third colorimetric band at this site after sample application indicates validity of the assay reaction on the devices. In further embodiments, an indication region/component overlaps at least in some area with the development region/component, or they share a common chromatographic substrate.

Non-limiting materials for indication regions/components include nitrocellulose membrane, UNISART CN95 membrane, CN 140 membrane and CN 150 membrane. In various embodiments, the chromatographic substrate is CN95 membrane for detecting sFlt-1 in a biological sample of saliva, plasma or whole blood.

In various embodiments, the chromatographic substrate is about 25 mm in length. In various embodiments, the chromatographic substrate is about 22-27 mm in length. In various embodiments, the chromatographic substrate is about 20-30 mm in length. In various embodiments, the chromatographic substrate is about 15-20 mm in length. In various embodiments, the chromatographic substrate is about 25-30 mm in length. In various embodiments, the chromatographic substrate is about 30-35 mm in length. In various embodiments, the chromatographic substrate is about 10-15 mm in length. In various embodiments, the chromatographic substrate is about 15-25 mm in length.

A wicking component/region, also referred to as a wick or when in the shape of a pad, a wicking pad, is generally a material to facilitate drawing of a liquid/fluid through the chromatographic substrate, or the development region/component and the indication region/component. A wick is typically placed at the end flow region of a strip/dipstick shaped device or on the underside of a cassette structured device. Non-limiting examples of useful materials for the wicking component/region include Grade 243 and Grade 319 pad, available from Ahlstrom Corporation.

In various embodiments, the wicking component/region is about 22 mm in length. In various embodiments, the wicking component/region is about 20-25 mm in length. In various embodiments, the wicking component/region is about 20-30 mm in length. In various embodiments, the wicking component/region is about 25-30 mm in length. In various embodiments, the wicking component/region is about 20-25 mm in length. In various embodiments, the wicking component/region is about 15-20 mm in length. In various embodiments, the wicking component/region is about 35-40 mm in length.

In various embodiments, the lateral flow assay further comprises a separation component to separate red blood cells from whole blood. In various embodiments, separation component can be placed between the sample receiving component/region and the development region/component and therefore the red blood cells are separated before the remainder of the sample moves into the development region/component. In other embodiments, separation component can be placed above the sample receiving component/region and therefore the red blood cells are separated before the remainder of the sample moves into the development region/component. In other embodiments, separation component can be placed below the sample receiving component/region and therefore the red blood cells are separated before the remainder of the sample moves into the development region/component.

Orientations

In some embodiments, liquid in the biological samples carrying analytes moves from the chromatographic substrate to a wick or absorbent pad. In some embodiments, the sample receiving region/component and the development region/component may be connected. In other embodiments, the sample receiving region/component and the development region/component may be combined. In further embodiments, the development region/component and the indication region/component may be combined. In some embodiments, at least a portion of the pad in the sample receiving region/component and the pad in the development region/component, or the combined pad, overlap(s) the chromatographic substrate. In some embodiments, the pad in the sample receiving region/component and the pad in the development region/component, or the combined pad, and the wicking pad are not connected. In some embodiments, the wicking pad can be separated from the chromatographic membrane by an impermeable membrane, except for the area where the wicking pad overlaps a portion of the chromatographic membrane.

In some embodiments, the indication region/component of the devices can be configured to direct flow of a liquid through the indication region/component in a generally horizontal orientation, e.g., substantially along a single horizontal plane from a first end of the indication region/component to the second end of the indication region/component. In other embodiments, the indication region/component can be configured to direct flow of a liquid through the indication region/component in a generally vertical orientation, e.g., substantially through a plurality of vertical planes, i.e., from the bottom of the indication region/component to the top of the indication region/component; or from the top of the indication region/component to the bottom of the indication region/component. In some embodiments, the indication region/component can be configured to split the flow of a liquid through the indication region/component into multiple paths. In some embodiments, the liquid may flow along from a first path to a second path that is curved, straight, or substantially parallel to the first path.

In some aspects, the sample receiving region is a component that is detachable from (and re-connectable to) the immunoassay region of the devices. In one example, the sample receiving region as a detachable component is an absorbent pad, and saliva produced by a subject (e.g., by unstimulated passive drooling) is collected by placing the absorbent pad under a subject's tongue for about 5 minutes or an another time such as about 7 minutes, 6 minutes, 4 minutes, 3 minutes, 2 minutes, 1 minute or 30 seconds so as to collect a sufficient amount of saliva for the assay. The absorbent pad or the sample receiving component containing the absorbent pad is then inserted into the immunoassay region, and the saliva enters (be fluidically connected to) the immunoassay region by, for example, capillary force. Salivary sFlt-1 (and/or one or more of its isoforms) is then detected and measured by a semi-quantitative lateral flow immunochromatographic assay in the immunoassay region.

In some embodiments, a device is a strip (e.g., lateral flow device) or dipstick including 1) an absorbent pad for collecting a biological sample (as a sample receiving region/component); 2) a conjugate pad imbedded with unbound purified monoclonal signal antibodies against sFlt-1 (and/or it one or more of its isoforms) which have been labeled with intensely colored gold nanoparticles and nonbinding monoclonal control antibodies labeled with gold nanoparticles of a different color (as a development region/component); (3) a membrane pad for passive diffusion of biological sample and conjugate antibodies, allowing time for binding reaction between salivary sFlt-1 and anti-sFlt-1 antibodies; (4) a test pad coated with a covalently-linked capture antibodies raised against sFlt-1; (5) a control line coated with covalently-linked capture antibodies raised against the monoclonal IgG control antibodies; ((3)-(5) as a chromatographic substrate in the indication region/component); (6) a wicking pad to draw a fixed amount of sample fluid or buffer across all sections of the immunoassay module. With this device, salivary sFlt-1 (and/or any one or more of its isoforms) is bound by the gold-labeled signal antibodies which will then be captured and accumulate on the test pad, producing a colored band. In other embodiments, the gold-labeled signal antibodies can be substituted with an alternative-labeled signal antibodies.

A further embodiment of the device, preferably in the form of a strip (e.g., lateral flow device) or dipstick, provides that the sample receiving region/component is an absorbent pad prepared with Ahlstrom Grade 1281 pad/paper, the development region/component is a conjugate pad prepared with Alhstrom Grad 6614 pad/paper, the indication region/component contains a nitrocellulose membrane, e.g., CN95 membrane selected, and the end flow region/component is a wicking pad prepared with Ahlstrom Grade 243 pad/paper. This device can be used for detecting sFlt-1 in plasma samples of a subject. This device can also be used for detecting one or more isoforms of sFlt-1 in plasma samples of a subject.

In various embodiments, the devices provide a “thermometer” type result, with the length of the zone that is saturated and colored by the antibody/antigen/antibody complex being proportional to the concentration of the antigen and the length of this colored zone will thus reflect a semi-quantitative assessment of the concentration. In one aspect of the exemplary strip/dipstick device above, the test pad is coated with a fixed and limited amount of capture antibody that is titrated, such that samples with low concentrations of Flt-1 will be bound almost entirely at the front end of the test pad and samples with moderate concentrations of Flt-1 will result in saturation of the capture antibody at the front of the test band, and additional Flt1-1 will be captured and accumulate farther down the test pad. The width of the test band colored by the antibody/Flt-1/antibody sandwich is proportional to the concentration of Flt-1 in the sample, therefore visually indicating the amount of Flt-1, producing a semi-quantitative reaction. As the salivary sample diffuses past the test pad, the control antibodies will then encounter the control line, causing capture and accumulation of the colored control antibodies, producing a second colored band which confirms the successful diffusion of saliva and conjugate antibodies. Other biological samples can be used as well.

In other embodiments, the devices provide a threshold-based approach to generating signals to indicate a positive result that is above a threshold. Typically, these devices have a test line and a control line, where target molecules that are detectably labeled with antibodies or antibody fragments migrate to the test line area, which they are concentrated by being bound by embedded capture antibody or antibody fragment, or by functional groups that specifically bind and immobilize capture antibodies that have the target molecules captured.

The devices allowing for the semi-quantitative reaction are used in determining the risk of having or developing preeclampsia in the subject whose biological sample is tested. Various instances the devices are provided in a test kit which also includes a color chart that provides a scale of colors based on a standard curve of Flt-1 concentrations in the medium that a test biological sample is in. In one aspect, the concentration of Flt-1 from the biological sample above or exceeding a designated clinical threshold is interpreted as a positive result indicating a high risk of preeclampsia; and the concentration of Flt-1 not exceeding or below a designated clinical threshold is interpreted as a negative result, which does not indicate a risk of preeclampsia. FIG. 11 depicts an exemplary color scale for visual grading of the test lines for Flt-1 in a biological sample.

Additional features in various embodiments of the devices include recovery of the biological sample by centrifugation of a device, and recovered sample (e.g., saliva, plasma or whole blood, or urine) is re-tested for Flt-1 using quantitative immunoassays to verify or confirm the result from the semi-quantitative test with the device.

Further embodiments of the devices are provided in a kit with reagents provided below, such as one or more of antibodies against target molecules and against control, chase buffer, additives, containers, and instruction manuals.

Antibodies Against Target Molecules and Control

Detection and/or capture reagent are generally antibodies or antibody fragment that are specific to the target molecule, herein Flt-1. In various embodiments, the antibodies are immunoreactive with soluble Flt-1; and in some embodiments, the antibodies are immunoreactive with bound Flt-1, e.g., Flt-1 bound to its ligands—VEGF and PlGF. Various embodiments provide the antibodies can be utilized in reverse orientation, where antibodies modified with a detectable label are coined “detection antibody” and antibodies modified with functional groups that can be immobilized by related functional groups at certain area(s) of the indication region of the device are coined “capture antibody”. Several monoclonal anti-Flt-1 antibodies are tested for the immunochromatographic devices (lateral flow devices). Exemplary anti-Flt-1 antibodies suitable for modification with a detectable label or with a functional group such that the modified anti-Flt-1 antibodies can be immobilized in an area of the chromatographic substrate of the devices are, for a human subject's biological sample, anti-human sFlt1-14 or anti-human VEGFR-1 antibodies, such as anti-hVEGFR DuoSet capture or detection antibody.

Some antibodies are lyophilized with one or more stabilizing additives, such as trehalose, sucrose, or a combination of both. In various embodiments, one or more antibodies in the presence with additives such as trehalose require curing to fully immobilize onto a membrane (e.g., nitrocellulose membrane). In some aspects, the membrane as the porous material of the devices is cured, e.g., through the exposure of a membrane to a high temperature for a duration of time (e.g., 45° C. for 1-12 hours or 1 day, 2 days or 3 days).

In some embodiments, the antibodies targeting the analyte (e.g., Flt-1) are polyclonal antibodies; where in some aspects, the detectable label modified antibody is a polyclonal antibody. In some embodiments, the antibodies targeting the analyte (e.g., Flt-1) are monoclonal antibodies; where in some aspects, the detectable label modified antibody is a monoclonal antibody. In some embodiments, the antibodies targeting the analyte (e.g., Flt-1) are a combination of a polyclonal antibody and a monoclonal antibody. Monoclonal anti-Flt-1 antibodies are in some embodiments suitable for being a component in the immunochromatographic devices. Polyclonal anti-Flt-1 antibodies are in other embodiments suitable for being a component in the immunochromatographic devices. In further embodiments, a combination of monoclonal and polyclonal anti-Flt-1 antibodies are used as the detection antibody and the capture antibody. Various aspects of the devices contain one type of antibody (polyclonal or monoclonal) selected based on the binding capability to the analyte target after the antibody is modified with a detectable label, e.g., the detectable-labeled antibody (such as colored latex particle-modified, polyclonal anti-VEGFR-1 antibody) remain at least 100%, 99%, 98%, 97%, 96%, 95%, 94%, 93%, 92%, 91%, 90%, 80%, 70%, 60% or 50% of the binding capability (e.g., measured by dissociation constant) of the antibody before it is modified with the detectable label.

Control antibodies are in various embodiments included in the devices. The indication region/component of the devices can comprise a first indicator and a second indicator. In some aspects, the second indicator signals the presence of the target molecule, e.g., Flt-1 (in an amount above a threshold or cut-off value designed with the devices). In some aspects, the first indicator and the second indicator are complementary. When both the first indicator and the second indicator provide an indication, a joint indication is provided to a user. The first indicator can be, for example, a control. The control can indicate that the detection means has been sufficiently exposed to a medium. In one aspect, the first indicator can signal a portion of at least one of a line, word, symbol, or character and the second indicator signals a different portion of the at least one of a line, word, symbol, or character. In another aspect, the first indicator can signal a first line, word, symbol, or character and the second indicator signals a different line, word, symbol, or character. In one aspect, the first indicator is intended as control, the second indicator is intended for target molecule Flt-1, and the signal of only the control indicator without a signal of the target molecule indicator signals to a user the absence or undetectability of the target molecule, whereas a joint signal of both the first indicator and the second indicator signals to a user the presence or detectability of a target molecule. Wherein only a signal of the target molecule indicator is visible bot not the control indicator, a user is advised to repeat the assay in another device or in another assay.

In some embodiments, for samples obtained from bodily fluid of a subject, control antibodies that bind to a housekeeping molecule in the sample such as immunoglobulin G are suitable for the assay or for the devices. In some embodiments, the control antibodies do not cross-react with the target molecule, Flt-1.

Detectable Label and/or Linkable Functional Groups to Modify Antibodies

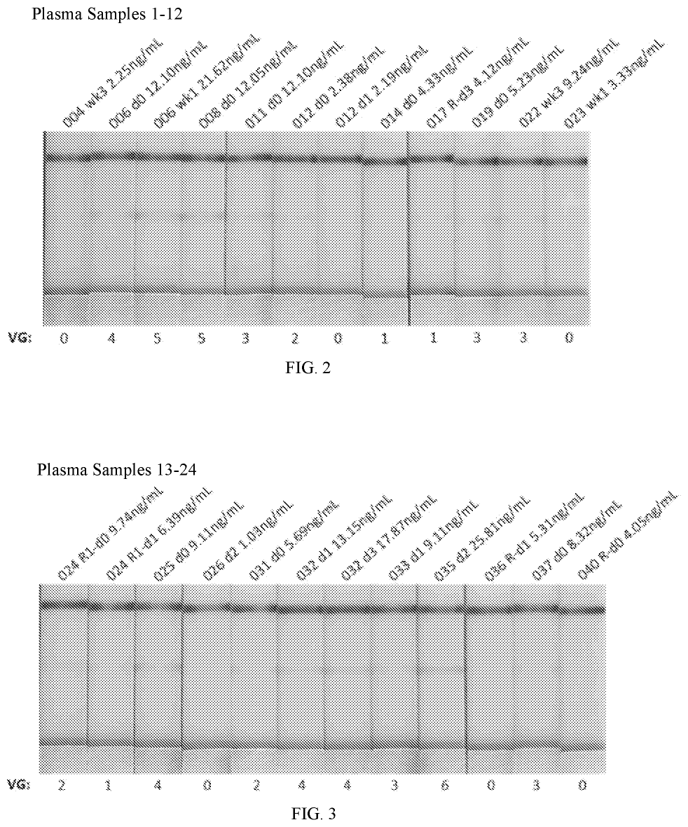

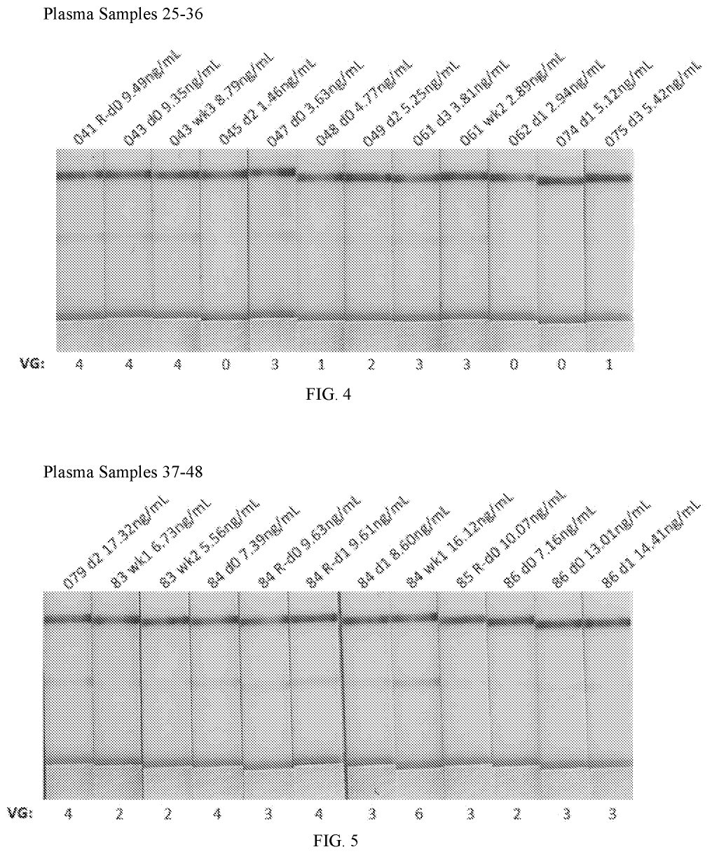

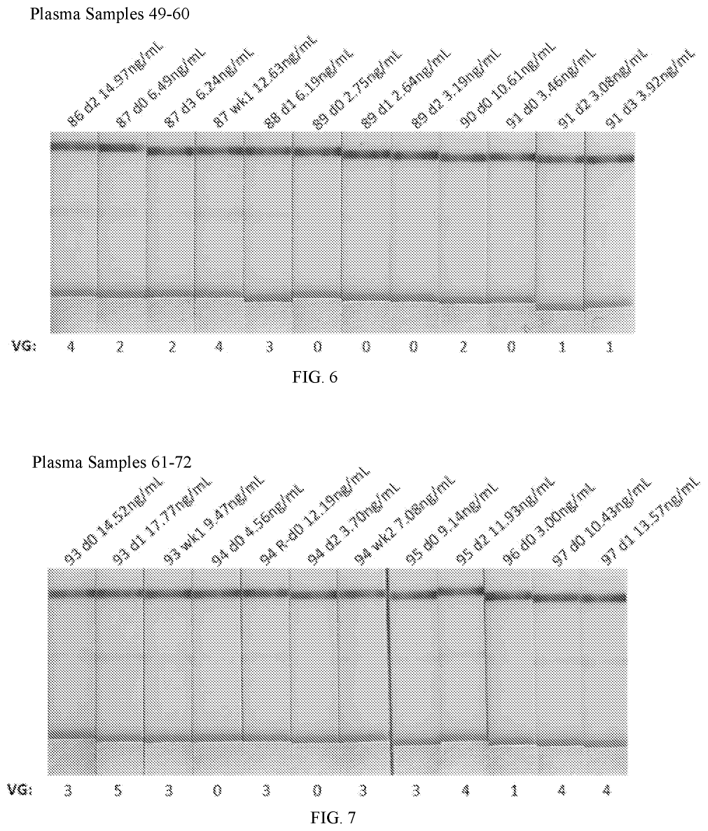

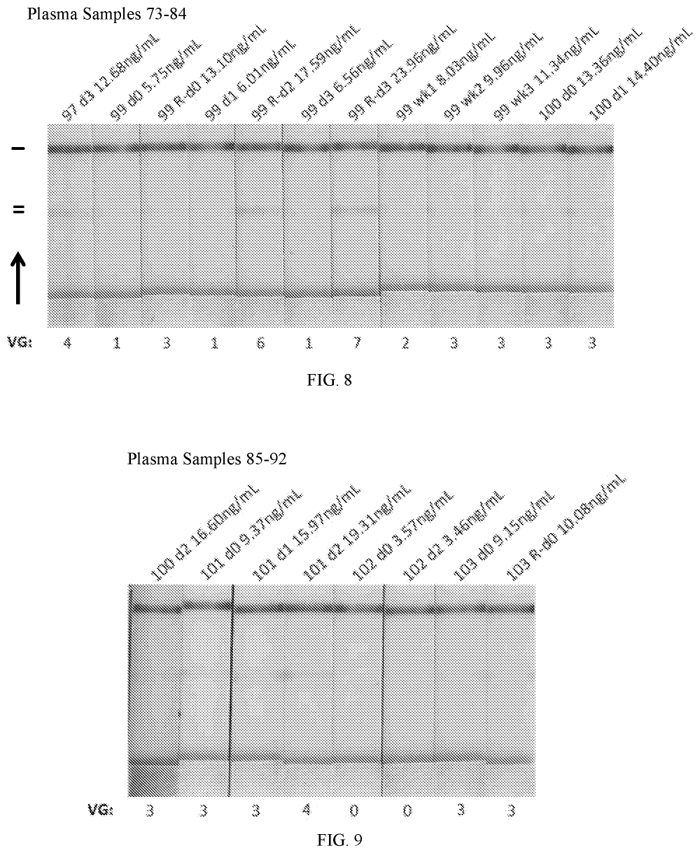

Various embodiments provide that the immunochromatographic devices (e.g., lateral flow devices) are intended for users to conduct assays and interpret visually the assay results without the aid of a digital reader, although the assay results can still be read via a densitometer or a reader. When detectable label-modified anti-Flt-1 antibodies bind with Flt-1, the signal of the detectable label represents the presence of Flt-1; the intensity of the detectable label signal indicates the concentration of Flt-1. In embodiments of “thermometer”-type approach to generating signal, e.g., where an anti-sFlt-1 antibody is evenly immobilized continuously across a length along the lateral flow direction in an indication region of the immunochromatographic devices, the length of the detectable label signal correlates with the amount of Flt-1, especially when the migration of the complex formed between Flt-1 and the detectable label-modified anti-Flt-1 antibody (“detection” antibody) is slow enough to allow for sufficient contact between the complex and the immobilized anti-Flt-1 antibody (“capture” antibody) and for the complex to “occupy” (bind and saturate) the immobilized antibody as the flow progresses. In embodiments of threshold-based approach of generating signal, e.g., exemplified in FIGS. 2-9 and Examples, a higher concentration of the target molecule leads to more deposition of the complex formed from the target molecule and its detection antibody to an area of indicator where capture antibody is immobilized, therefore the intensity of the signal of the indicator correlates to the concentration of the target molecule in the biological sample. Typically, the devices contain at least equal or a greater amount of capture antibody than the detection antibody.

In various embodiments, detectable labels are visually detectable labels such as colloidal gold, cellulose nanobeads, colored latex particles, and nanoshells (silica core with functionalized gold shell). In some embodiments, detectable labels are one or more metal nanoparticles, such as gold nanoparticles, or quantum dots. In other embodiments, detectable labels are one or more of a liposome filled with a colored substance, an enzyme, a radiolabel, chromophore and a fluorophore. One or more of the detectable labels are modified onto antibodies against Flt-1. In some embodiments, the devices contain one or more anti-Ftl-1 antibodies that are modified with colored latex particles in a number ratio between the latex particle and the antibody of at least 1:1, 5:1, 10:1, 15:1, 20:1, 25:1, 30:1, 35:1, 40:1, 45:1, 50:1, 60:1, 70:1, 75:1, 80:1, 90:1, or 100:1. In one embodiment, the anti-Flt-1 antibodies in the devices are not modified with gold nanoparticles or nanoshells. In some embodiments, the detectable labels are viewable with visual examination. In other embodiments, detectable labels are visible or quantifiable with the aid of digital readers or other equipment.

Detectable labels are modified onto analyte-binding antibodies via one or more of chemical and/or physical conjugation/bonding techniques to form detection antibodies. In some instances, pairs of functional groups are utilized, where one reactive functional group is modified onto the analyte-binding antibody and the corresponding reactive functional group is modified onto the detectable label, such that the detectable label comes in contact and is conjugated with the analyte-binding antibody. Preferably, the reactive chemical functional groups for the conjugation between the analyte-binding antibody and the detectable label do not cross react with the analyte, in order to avoid interference of the binding between analyte and analyte-binding antibody. Also preferably, the reactive chemical functional groups and their amounts do not cause aggregation, or less than 5%, 6%, 7%, 8%, 9% or 10% aggregation of the conjugated complexes.

A functional group can also be modified onto an analyte-binding antibody to form a capture antibody, wherein the functional group modified onto the analyte-binding antibody is capable of binding with another functional group that is present in an indication area of the devices, such that the capture antibody is immobilized in the indication area. In some embodiments, capture antibodies are present in the development region/component of the devices, and the capture antibodies bind with the target molecule (Flt-1) and the detection antibodies also bind with the target molecule, forming a “sandwich” complex, followed by the sandwich complex migration to the indication region/component of the devices, where the complex is immobilized/deposited at the indication area. In other embodiments, capture antibodies are pre-immobilized at the indication area of the devices, and the target molecule or a detection antibody-bound target molecule migrates to the indication area and be bound by the capture antibody.

Exemplary functional group modifications for conjugation include pairs of functional groups such as streptavidin and biotin, polystreptavidin and biotin, neutravidin and biotin, and avin and biotin.

Additives

Various embodiments provide the devices utilize one or more additives with the reagents or embedded with one or more regions of the porous materials. Exemplary additives include but are not limited to carrier proteins, surfactants (such as amphiphilic molecules), salts, or buffers containing carrier proteins, surfactants or salts. In one embodiment, additive includes TRIS buffer, magnesium chloride, choline chloride, or a combination thereof. In another embodiment, the devices contain a detectably labeled anti-VEGFR-1 antibody in the presence with one of the three additives selected from the group consisting of TRIS buffer, magnesium chloride, and choline chloride.

Chase Buffer (or Chase Solution)

Various embodiments provide the devices utilize one or more chase buffers. In some embodiments, chase buffer is used to facilitate wicking of samples in the devices. In one aspect, sample is added to the sample receiving region of the devices first, then the device (its sample receiving region) is submerged in chase buffer, and capillary force facilitates the flow of the chase buffer carrying sample molecules into and/or across the development region. Other embodiments provide that chase buffer is used to mix with and/or pre-dilute samples, and the chase buffer-sample is then added to the sample receiving region of the devices. Further embodiments provide biological sample is directly added to the sample receiving region of the devices, and no chase buffer is subsequently added to the devices.

Exemplary chase buffers are water-based buffer solution with one or more additives. For example, a chase buffer is PLURONIC F127 in phosphate-buffer saline, such as 0.75% PLURONIC F127 in phosphate buffered saline, or another concentration of PLURONIC polymers like about 0.1%, 0.2%, 0.3%, 0.4%, 0.5%, 0.6%, 0.7%, 0.8%, 0.9%, 1%, 2%, 3%, 4%, 5%, or any range in between any two of the listed amounts. In some embodiments, other PLURONIC poloxamers are included in the chase buffer, including but are not limited to PLURONIC F68, poloxomer 407, PLURONIC P85, and PLURONIC F108.

Thresholds

Generally, a minute amount of Flt-1 is present in the plasma, saliva, and/or whole blood of a non-pregnant female, or in a pregnant female not having preeclampsia, not prone to develop preeclampsia and not exhibiting symptoms of preeclampsia. This amount of “normal” presence of Flt-1 is in some embodiments less than 5 ng/mL. In other embodiments, the amount of “normal” presence of Flt-1 is in some embodiments less than 4 ng/mL. In other embodiments, the amount of “normal” presence of Flt-1 is in some embodiments less than 3 ng/mL. In other embodiments, the amount of “normal” presence of Flt-1 is in some embodiments less than 2.5 ng/mL. In other embodiments, the detection limit of a lateral flow device disclosed herein is above 5.5 ng/mL, 6 ng/mL, 6.5 ng/mL, 7 ng/mL, 7.5 ng/mL or 7.25 ng/mL in undiluted biological samples.

Cut-off values, also referred to designated clinical threshold, for the semi-quantitative assay in the devices are designated for assigning a positive value when the concentration of the analyte in a sample exceeds the cut-off value to indicate a predisposition or high likelihood to develop the symptom or condition that the analyte is associated with, or for assigning a negative value when the concentration of the analyte in a sample is below the cut-off value to indicate a low likelihood or no sign of developing the symptom or condition that the analyte is associated with. In many instances, cut-off values for the provided devices herein are determined based on, or within 5%, 10%, 15% or 20% larger than, a “normal” amount of sFlt-1 in the respective source of samples in an average non-pregnant female or pregnant female not having preeclampsia (or the average “normal” amount in a population of non-pregnant females or pregnant females not having preeclampsia). In some embodiments, the cut-off values are 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75 or 10 ng/mL. In some particular embodiments, the cut-off value is 5 ng/mL. In some particular embodiments, the cut-off value is 4 ng/mL. In some particular embodiments, the cut-off value is 8 ng/mL. In some particular embodiments, the cut-off value is 7 ng/mL. In some particular embodiments, the cut-off value is 6 ng/mL. In some embodiments, the devices are configured to or is accompanied by a color chart that can indicate an analyte concentration that is at least 5% higher than the cut-off value. In some embodiments, the devices are configured to or is accompanied by a color chart that can indicate an analyte concentration that is at least 5% lower than the cut-off value.

In some embodiments, wherein the measuring the analyte includes using a densitometer, the cut-off value is 1000 Units. In various embodiments, the cut-off value is 100 Units, 200 Units, 300 Units, 400 Units, or 500 Units. In various embodiments, the cut-off value is 600 Units, 700 Units, 800 Units, or 900 Units. In various embodiments, the cut-off value is 450 Units. In various embodiments, the cut-off value is 500 Units. In various embodiments, the cut-off value is 540 Units. In various embodiments, the cut-off value is 550 Units. In various embodiments, the cut-off value is between 100 Units and 200 Units. In various embodiments, the cut-off value is between 200 Units and 300 Units. In various embodiments, the cut-off value is between 300 Units and 400 Units. In various embodiments, the cut-off value is between 400 Units and 500 Units. In various embodiments, the cut-off value is between 500 Units and 600 Units. In various embodiments, the cut-off value is between 600 Units and 700 Units. In various embodiments, the cut-off value is between 700 Units and 800 Units. In various embodiments, the cut-off value is between 800 Units and 900 Units. In various embodiments, the cut-off value is between 900 Units and 1000 Units. In various embodiments, the cut-off value is between 1000 Units and 1100 Units. In various embodiments, the cut-off value is between 1100 Units and 1200 Units. In various embodiments, the cut-off value is between 1200 Units and 1300 Units. In various embodiments, the cut-off value is between 1300 Units and 1400 Units. In various embodiments, the cut-off value is between 1400 Units and 1500 Units.

In various embodiments, the cut-off value chosen for a densitometer can be chosen to correlate the densitometer units to a cut-off value that is a concentration of any one or more isoforms of Flt-1 or fragments thereof (soluble or membrane bound). In various embodiments, the cut-off value chosen for a densitometer can be chosen to correlate the densitometer units to a cut-off value that is a concentration of any one or more isoforms of Flt-1 or fragments thereof (soluble or membrane bound), and the development/run time on the lateral flow device of the present invention; for example, about 5-25 minutes; particularly, it can be about 5, 10, 15, 20, or 25 minutes. Particular embodiments can be about 15 minutes.

In various embodiments, the immunochromatographic devices (or lateral flow device) disclosed herein are configured to allow for a cut-off value at 7.5 ng/mL of Flt-1 for an assay sample obtained from plasma or whole blood of a subject, where a concentration of Flt-1 equal or greater than 7.5 ng/mL is indicated positive in the device for risk of preeclampsia of the subject.

In another embodiment, the immunochromatographic devices (or lateral flow device) disclosed herein are configured to allow for a cut-off value at 5 ng/mL of Flt-1 for an assay sample obtained from saliva of a subject, where a concentration of Flt-1 equal or greater than 5 ng/mL is indicated positive in the device for risk of preeclampsia of the subject.

In various embodiments, the cut-off value can be determined based on sensitivity of the assay. Thus, a lower cut-off value can be utilized for assays that have higher sensitivities.

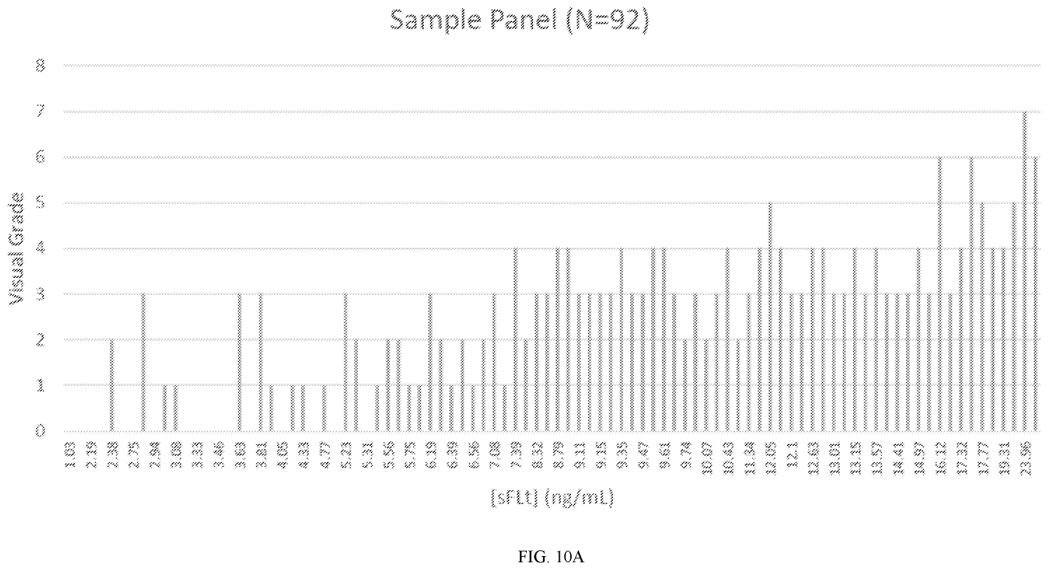

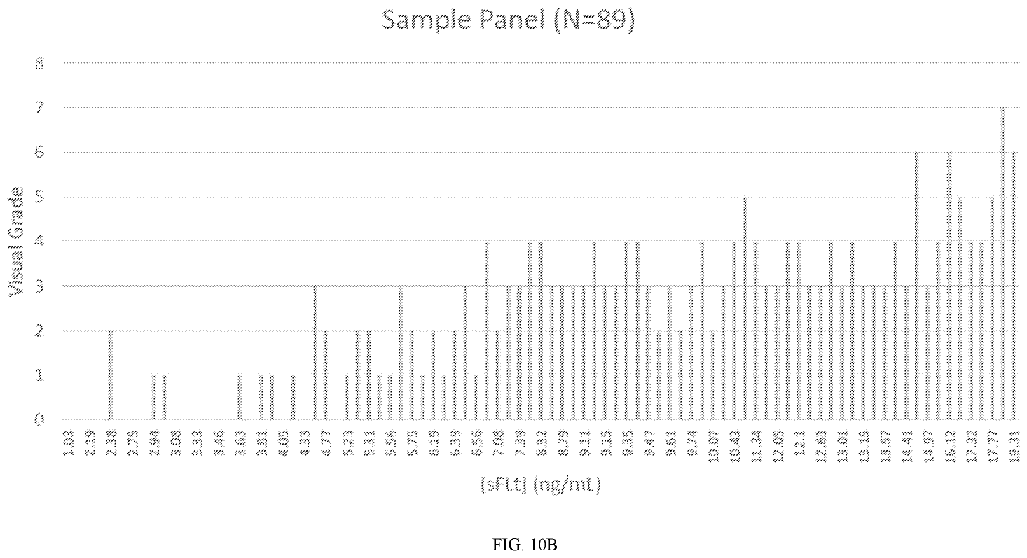

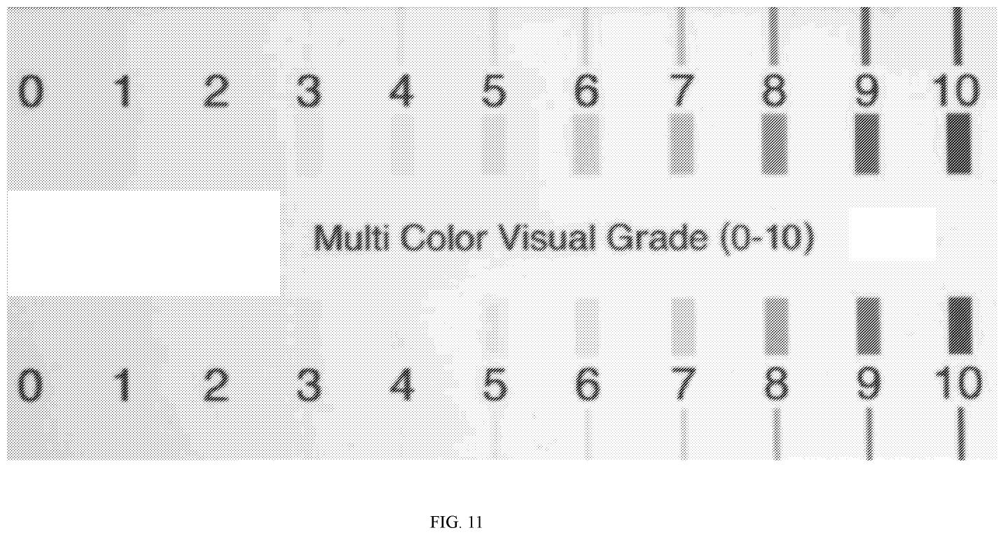

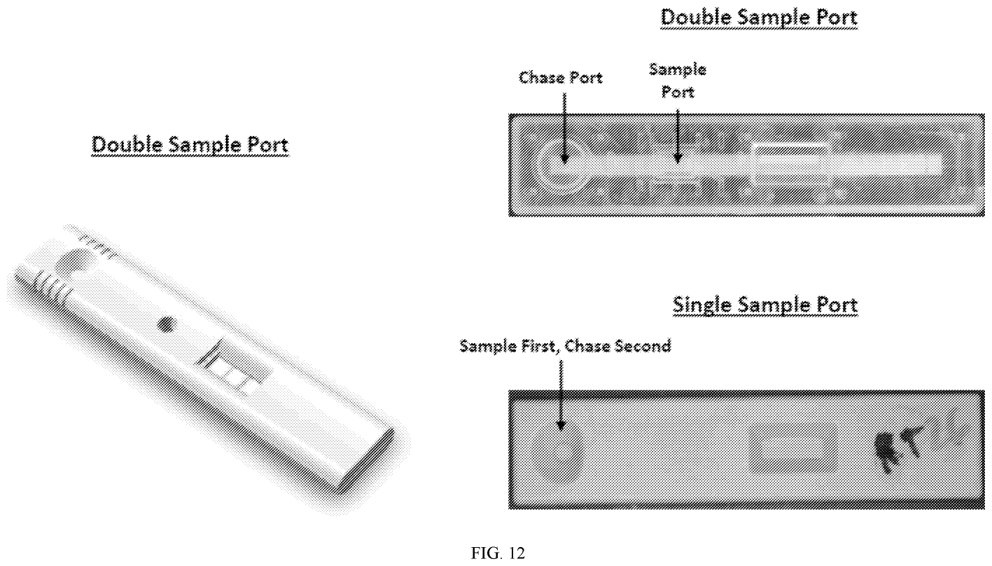

The devices are suitable for use in various environments including but are not limited to medical practitioner's office, clinical laboratory, home use, central hospital laboratory, pharmacy, and other point-of-care testing facilities or environments. One example of intended users of the devices is expectant mothers, who can use the devices at home. An exemplary format of the devices is a strip that can be used in a dipstick or lateral flow immunoassay manner for assays. Another exemplary format of the devices is a cassette, such as an off-the-shelf cassette. An example of the cassette is shown in FIG. 12.

Sensitivity

Various embodiments of the devices provide that the devices are configured to discern an analyte concentration, e.g., concentration of Flt-1 including any isoforms thereof in the biological sample, that is at least above 7 ng/mL, above 7.5 ng/mL, above 8 ng/mL or above 9 ng/mL. For example, the devices are configured to provide for different visual grades for analyte amounts comparing 7 ng/mL and 8 ng/mL, or comparing 7 ng/mL and 9 ng/mL or greater. In further aspects of these embodiments, the devices are configured to provide negative indication of the analyte if the analyte is not present or less than 3 ng/mL in the biological sample; and provide positive indication of the presence of the analyte as well as its quantity based on intensity of the detectable label in the indication region of the analyte.

Other embodiments provide that the devices have a sensitivity of at least 1 ng/mL, 2 ng/mL, 3 ng/mL, 4 ng/mL, or 5 ng/mL. In still other embodiments, the devices have a sensitivity of at least 3 ng/mL, 4 ng/mL, 5 ng/mL, or 6 ng/mL. In still other embodiments, the devices have a sensitivity of at least 6 ng/mL, 7 ng/mL, 8 ng/mL, 9 ng/mL, or 10 ng/mL. In still other embodiments, the devices have a sensitivity of at least 11 ng/mL, 12 ng/mL, 13 ng/mL, 14 ng/mL, or 15 ng/mL. In still other embodiments, the devices have a sensitivity of at least 20 ng/mL, 25 ng/mL, 30 ng/mL, 40 ng/mL or 50 ng/mL. The sensitivity of the devices is in various embodiments a threshold above which the presence of the analyte is visually detectable. The sensitivity or discernibility of the devices on sFlt-1 is commonly related to the antibody, the functional group in the conjugation with detectable label, or both. For example, polystreptavidin as the functional group in the conjugation between a detectable label and an anti-sFlt-1 antibody shows a very strong separation between 0 ng/mL and 30 ng/mL spiked antigen. In some embodiments, a separation so strong that a minute amount (e.g., 2 ng/mL and below) will show a visibly strong test line/indication is not desirable. In further aspects, samples are appropriately tested positive and even semi-quantitatively (detectable label intensity in correlation with the amount of analyte) with the devices containing about 0.1 mg/mL or ±1 order of magnitude of polystreptavidin, about 2 mg/mL or ±1 order of magnitude of streptavidin. In some embodiments, a 0.1 mg/mL polystreptavidin test line provides a visual cut off around 4 ng/mL of the analyte at 10 minutes while 2.0 mg/mL streptavidin provides a visual cut off around 9 ng/mL at 10 minutes and a cutoff of approximately 4 ng/mL at 15 minutes.

In various embodiments, the devices in configured for, or supports, a lateral flow assay which is composed of a sample receiving region, optionally a development region, an indication region, and the end-flow/wicking region, and the lateral flow assay can have a length of less than 100 mm, a width of less than 6 mm, and a thickness of less than 2.0 mm. In various embodiments, the lateral flow assay can have a length of less than 90 mm, a width of less than 5 mm, and a thickness of less than 1.6 mm. In various embodiments, the lateral flow assay can have a length of about 84 mm, a width of about 4 mm, and a thickness of less than 1.57 mm. In various embodiments, the lateral flow assay can have a length of about 84 mm, a width of about 4 mm, and a thickness of less than 1.55 mm. In various embodiments, example, the length of the lateral flow assay can be about 90 mm, 89, mm, 88 mm, 87 mm, 86 mm, 85 mm, 84 mm, 83 mm, 82 mm, 81 mm, or 80 mm. In other examples, the width of the lateral flow assay can be about 6 mm, 5 mm, 4 mm, 3 mm, 2 mm, or 1 mm. Various combinations of the aforementioned length and width are encompassed by embodiments of the present invention.

In various embodiments, the devices in configured for, or supports, a lateral flow assay which is composed of a sample receiving region, optionally a development region, an indication region, and the end-flow/wicking region, and the lateral flow assay can have a length of less than about 12 mm, a width of less than about 6 mm, and a thickness of less than about 1.5 mm. In some embodiments, the lateral flow assay can have a length of about 10 mm, a width of about 4 mm or less, and a thickness of about 1 mm or less. In some embodiments, the lateral flow assay can have a length of about 10 mm, a width of about 3 mm or less, and a thickness of about 1 mm or less. For example, the length of the lateral flow assay can be about 24 mm, 23, mm, 22 mm, 21 mm, 20 mm, 19 mm, 18 mm, 17 mm, 16 mm, 15 mm, 14 mm, 13 mm, 12 mm, 11 mm, 10 mm, 9 mm, 8 mm, 7 mm, 6 mm, or 5 mm. In other examples, the width of the lateral flow assay can be about 6 mm, 5 mm, 4 mm, 3 mm, 2 mm, or 1 mm. Various combinations of the aforementioned length and width are encompassed by embodiments of the present invention.

Devices disclosed herein are provided to allow for sensitivity and specificity of the assay and to be able to distinguish around a defined positive/negative threshold reproducibly in a semi-quantitative (threshold) assay format.

The devices in various embodiments are self-contained, disposable, single-use devices. Under various conditions, the devices are used for conducting an assay and providing visual result within 15 minutes, 14 minutes, 13 minutes, 12 minutes, 11 minutes, 10 minutes, 9 minutes, 8 minutes, 7 minutes, 6 minutes, 5 minutes, 4 minutes, 3 minutes, 2 minutes or 1 minute. In various instances, the visual indication and result of the assay on the devices are stable for at least 10 minutes, 15 minutes, 20 minutes, 30 minutes, 40 minutes, 50 minutes or 60 minutes after the assay is completed, such that in methods of using the devices to determine likelihood or unlikelihood of having preeclampsia, results in the indication region can be read or visually examined immediately after the assay is completed or, even re-read or re-examined in about 10 minutes, 15 minutes, 20 minutes, 30 minutes, 40 minutes, 50 minutes or 60 minutes after the assay is completed. In many instances, the devices have a shelf-life of about one month, two months, three months, four months, or longer, or at least two months, when stored at room temperature, preferably in a sterile package. In other embodiments, the devices have a shelf-life of about five months, 6 months, 7 months 8 months, 9 months 10 months, 11 months, 12 months or longer. In other instances, the devices are also stable and can be stored in refrigeration (e.g., at 4° C.) for about 6, 7, 8, 9, 10, 11, 12, 13, 14, 15, 16, 17 or 18 months, or at least for 12 months. In other instances, the devices are also stable and can be stored in refrigeration (e.g., at 4° C.) for about 1 year, 1.5 years, 2 years, 2.5 years, 3 years or longer.

The devices in various embodiments have a dimension or size that is portable. In one embodiment, the devices or at least the sample receiving region of the devices are configured to placement into the mouth of a human, and/or underneath the tongue, such that saliva is in contact with the sample receiving region of the devices.

Various aspects of the devices utilize a biological sample of a volume of about 10 μL, 15 μL, 20 μL, 30 μL, 40 μL, 50 μL, 100 μL, 200 μL, 300 μL, 400 μL, 500 μL, 1 mL, 2 mL, 3 mL, 4 mL, 5 mL or 10 mL of a sample. In some aspects, a device requires 10-20 μL, 20-30 μL, 30-40 μL, 40-50 μL, 50-100 μL, 100-200 μL, 200-300 μL, 300-400 μL, 400-500 μL, 500 μL-1 mL, 1-2 mL, 2-3 mL, 3-4 mL, 4-5 mL, or 5-10 mL.

In particular embodiments, a lateral flow device includes a sample receiving region, optionally a development region, an indication region, and the end-flow/wicking region, wherein the lateral flow device can have a length of less than 84 mm (or no greater than 84 mm), a width of less than 6 mm (or no greater than 6 mm), and a thickness of less than 1.6 mm (or no greater than 1.6 mm), which supports or is configured for lateral flow assaying of a biological sample of about 15 μL (or at least 10 μL), wherein the assaying positively detects Flt-1 (e.g., sFlt-1) of at least 7.5 ng/mL in a substantially undiluted biological sample obtained from a human subject (or equivalent to at least 7.5 ng/mL in substantially undiluted human plasma, e.g., 15 ng Flt-1/mL of 2-fold diluted human plasma, 30 ng Flt-1/mL of 4-fold diluted human plasma).

In particular embodiments, a lateral flow device includes a sample receiving region, optionally a development region, an indication region, and the end-flow/wicking region, wherein the lateral flow device can have a length of less than about 12 mm (or no greater than 12 mm), a width of less than about 6 mm (or no greater than 6 mm), and a thickness of less than about 1.5 mm (or no greater than 1.5 mm), which supports or is configured for lateral flow assaying of a biological sample of about 15 μL (or at least 10 μL), wherein the assaying positively detects Flt-1 (e.g., sFlt-1) of at least 7.5 ng/mL in a substantially undiluted biological sample obtained from a human subject (or equivalent to at least 7.5 ng/mL in substantially undiluted human plasma, e.g., 15 ng Flt-1/mL of 2-fold diluted human plasma, 30 ng Flt-1/mL of 4-fold diluted human plasma).

Further embodiments provide methods of manufacturing a lateral flow device for detecting fms-like tyrosine kinase 1 (Flt-1) protein fragments or isoforms including sFlt1 isoforms. The methods include: (1) providing a base and providing a substrate positioned above the base, the substrate defining: a sample receiving region, an indication region, and optionally a development region positioned between (or overlapping with each of) the sample receiving region and the indication region, each region comprises a porous material and is in capillary contact with at least one other region, thereby permitting a fluid to wick from the sample receiving region to the indication region; (2) immobilizing a first capture reagent or a modification capable of binding the first capture reagent at a first location in the indication region, wherein the first capture reagent comprises a monoclonal or polyclonal antibody specifically immunoreactive with Flt-1, or an antigen-binding fragment thereof; (3) providing a first detection reagent comprising a detectable label and an antibody or fragment thereof capable of binding the Flt-1, wherein the first detection reagent is capable of being transported from the development region to the indication region.

In some aspects, the sample receiving region is configured to receive a fluid sample containing one or more analytes and to permit the fluid sample to wick to the indication region, and when the analytes comprise Flt-1, a complex is formed comprising the first detection reagent, the Flt-1, and the first capture reagent, and the complex indicates the presence of the Flt-1 through the detectable label at the first location in the indication region.

In further aspects, the methods of manufacturing a lateral flow device further include (4) providing at a second location in the indication region a second capture reagent or a modification capable of immobilizing the second capture reagent, (5) providing a second detection reagent capable of being transported to the indication region, wherein the second detection reagent is capable of binding a house-keeping molecule in the fluid sample, and the second detection reagent, the house-keeping molecule and the second capture reagent form a complex to indicate a presence of the house-keeping molecule through the second detection reagent, wherein the second mobile detection reagent is not cross-reactive with Flt-1, with the first detection reagent or with the first capture reagent, and (6) further providing an end flow region comprising a porous material and positioned such that a fluid is conducted from the sample receiving region through the indication region.

In some embodiments, a lateral flow device is in the form of a strip, whose (1) indication region (e.g., membrane) at a first location is immobilized (e.g., striped, pre-striped, contacted, having been contacted, soaked, pre-soaked) with polystreptavidin using a polystreptavidin solution, and at a second location is immobilized (e.g., striped, pre-striped, contacted, having been contacted, soaked, pre-soaked) with goat anti-mouse antibody using a goat anti-mouse antibody solution; said polystreptavidin solution can be equivalent or about equivalent to containing the polystreptavin at 0.1-0.2 mg/mL in 1× phosphate buffered saline (PBS), 1.6 mM tris (hydroxymethyl) aminomethane (TRIS) (pH 8), and 2% sucrose, and said goat anti-mouse antibody solution containing the goat anti-mouse antibody at about 0.3 mg/mL in 1×PBS, 0.8 mM TRIS (pH 8), and 1% sucrose; optionally the first location and the second location are about 4-6 mm apart or further apart, separated in the direction of fluid flow across the length of the strip; (e.g., the first location is upstream to the second location); and whose (2) development region (conjugate pad) comprises latex particle-labeled DuoSet capture antibody as a detection reagent, said latex particle (e.g., colored latex particles) being conjugated to the DuoSet capture antibody at a mass ratio of about 35:1-45:1 (e.g., optionally the colored latex particle conjugate is equivalent or about equivalent to being prepared at 0.1% in a diluent of pH 8.5 containing 50 mM borate, 1% casein, 10% sucrose, 2% trehalose); optionally the detection reagent stays in the development region (conjugate pad) while the strip is dry, but is capable of binding a target molecule (e.g., Flt-1 or sFlt-1 (any isoforms thereof)) as well as being carried to the indication region when the strip is in fluid contact to support lateral flow; and (3) whose sample receiving region (sample pad) is pre-soaked or contacted with a solution containing MgCl₂ and PLURONIC F127 (e.g., pre-soaked or contacted with what is equivalent or about equivalent to 1M MgCl₂ in 10% PLURONIC F127), or whose sample receiving region (sample pad) comprises MgCl₂ and PLURONIC F127 in an effective amount for reducing binding of other molecules than target molecules (e.g., Flt-1 sFlt-1 (any isoforms thereof)); optionally pre-soaking or contacting the sample pad or sample receiving region with the solution containing the MgCl₂ and the PLURONIC F127 includes adding what is equivalent to or about equivalent to about 2.5 mL of the solution to a sample pad of a volume equivalent to or about equivalent to 300 mm×22 mm×a thickness of 2 mm or less, followed by drying the pad in about 40° C. for at least 45 minutes (e.g., in a forced air oven.)

In some embodiments, a lateral flow device is in the form of a strip, whose (1) indication region (e.g., membrane) at a first location is immobilized (e.g., striped, contacted, soaked) with polystreptavidin using a polystreptavidin solution, and at a second location is immobilized (e.g., striped, contacted, soaked) with goat anti-mouse antibody using a goat anti-mouse antibody solution; said polystreptavidin solution containing the polystreptavin at 0.1-0.2 mg/mL in 1× phosphate buffered saline (PBS), 1.6 mM tris (hydroxymethyl) aminomethane (TRIS) (pH 8), and 2% sucrose, and said goat anti-mouse antibody solution containing the goat anti-mouse antibody at about 0.3 mg/mL in 1×PBS, 0.8 mM TRIS (pH 8), and 1% sucrose; optionally the first location and the second location are about 5 mm apart or further apart, separated in the direction of fluid flow across the length of the strip, (e.g., the first location is about 47 mm from one end of the strip, and the second location is about 52 mm from the same end of the strip (both referenced from the beginning of the development region of the strip); e.g., the first location is upstream to the second location); and whose (2) development region (conjugate pad) comprises latex particle-labeled DuoSet capture antibody as a detection reagent, said latex particle (e.g., red latex particles (400 nm)) being conjugated to the DuoSet capture antibody at a mass ratio of about 40:1 (e.g., optionally the red latex particle conjugate is prepared at 0.1% in a diluent of pH 8.5 containing 50 mM borate, 1% casein, 10% sucrose, 2% trehalose); optionally the detection reagent stays in the development region (conjugate pad) while the strip is dry, but is capable of binding a target molecule (e.g., Flt-1 or sFlt-1) as well as being carried to the indication region when the strip is in fluid contact to support lateral flow; and (3) whose sample receiving region (sample pad) is pre-soaked or contacted with a solution containing MgCl₂ and PLURONIC F127 (e.g., pre-soaked or contacted with 1M MgCl₂ in 10% PLURONIC F127), or whose sample receiving region (sample pad) comprises MgCl₂ and PLURONIC F127 in an effective amount for reducing binding of other molecules than target molecules (e.g., Flt-1 sFlt-1); optionally pre-soaking or contacting the sample pad or sample receiving region with the solution containing the MgCl₂ and the PLURONIC F127 includes adding about 2.5 mL of the solution to a sample pad of a volume equivalent to 300 mm×22 mm×a thickness of less than 2 mm, followed by drying the pad in 40° C. for at least one hour (e.g., in a forced air oven.)

Detection and/or Diagnostic Assays and Methods

Elevated concentrations of soluble fms-like tyrosine kinase 1 (sFlt-1) or bound Flt-1 in salivary, plasma, urine, or other bodily fluids are associated with increased risk or presence of preeclampsia or its related disorders in pregnant mothers.

Assays are provided for detecting Flt-1 in a sample such as saliva, plasma, urine, or a bodily fluid from a subject using the devices disclosed herein. The assays include contacting the saliva, plasma, urine or another bodily fluid of the subject with the sample receiving region of the devices, and determining the presence or absence of a signal in the indication region where anti-Flt-1 antibody is immobilized, wherein the presence of a signal in the indication region where anti-Flt-1 antibody is immobilized detects the presence of Flt-1 in the sample, and the absence of a signal in the indication region where anti-Flt-1 antibody is immobilized indicates Flt-1 is not detected in the sample.

Assays are provided for detecting and quantifying Flt-1 in a sample such as saliva, plasma, urine, or a bodily fluid from a subject using the devices disclosed herein. The assays include contacting the saliva, plasma, urine or bodily fluid of the subject with the sample receiving region of the devices, and determining the presence or absence of a signal in the indication region where anti-Flt-1 antibody is immobilized, wherein the absence of a signal in the indication region where anti-Flt-1 antibody is immobilized indicates Flt-1 is not detected in the sample, and when a signal is present in the indication region where anti-Flt-1 antibody is immobilized, further comparing the signal to a standard to determine the level of Flt-1 in the sample. In various embodiments, the assays are intended for a pregnant woman, a pregnant woman at risk of hypertensive disorder, a pregnant woman at risk of preeclampsia and/or eclampsia, a pregnant woman having hypertensive disorder, or a pregnant woman having preeclampsia or eclampsia. In various embodiments, the assays are intended for postpartum women. In various embodiments, the assays are intended for postpartum women who are about 1 week, 2 weeks, 3 weeks, and/or 4 weeks postpartum. In various embodiments, the assays are intended for postpartum women who are about 1 month 2 months and/or 3 months post-partum. In various embodiments, the assays are intended for postpartum women who are more than 3 months postpartum.

In some embodiments, methods of assaying a biological sample are provided, using a lateral flow device described herein. In some embodiments, methods for detecting a level, or a presence or absence, of an analyte in the biological sample, are provided, wherein the analyte includes soluble fms-like tyrosine kinase 1 (sFlt-1), bound Flt-1, or both, using a lateral flow device described herein. The methods include, or consists of, applying the biological sample to the sample receiving region of the lateral flow device, so as to permit the biological sample to flow to the indication region; and detecting the level, or the presence or absence, of the first detectable label at a first location in the indication region of the lateral flow device; wherein the lateral flow device contain a first capture reagent that is specifically immunoreactive to the analyte, or a complex is formed of the first detection reagent, the analyte, and the first capture reagent.

In some embodiments, a method of selecting a pregnant human and assaying a biological sample from the pregnant human is provided, using a lateral flow device disclosed herein. In some embodiments, a method of selecting a pregnant human and detecting a level, or a presence or absence, of an analyte in the biological sample from the pregnant human is provided. The method includes, or consists of, (1) selecting a pregnant human about 20 weeks into gestation (or having about 20 weeks or after 20 weeks of pregnancy), or a pregnant human 18-20 weeks into gestation, (2) applying or contacting a biological sample (e.g., plasma or saliva or urine) obtained from the selected pregnant human with a lateral device disclosed herein (e.g., contacting the biological sample in the sample receiving region of a lateral flow device), and (3) detecting the level, or the presence or absence, of Flt-1 (e.g., sFlt-1) by measuring a detectable label in the indication region. In other embodiments, the method includes, or consists of (1) selecting a pregnant human about 20-30 weeks into gestation, (2) applying or contacting a biological sample (e.g., plasma or saliva or urine) obtained from the selected pregnant human with a lateral device disclosed herein (e.g., contacting the biological sample in the sample receiving region of a lateral flow device), and (3) detecting the level, or the presence or absence, of Flt-1 (e.g., sFlt-1) by measuring a detectable label in the indication region. In other embodiments, the method includes, or consists of (1) selecting a pregnant human about 30-40 weeks into gestation, (2) applying or contacting a biological sample (e.g., plasma or saliva or urine) obtained from the selected pregnant human with a lateral device disclosed herein (e.g., contacting the biological sample in the sample receiving region of a lateral flow device), and (3) detecting the level, or the presence or absence, of Flt-1 (e.g., sFlt-1) by measuring a detectable label in the indication region.

In some embodiments, a method of selecting a postpartum human and assaying a biological sample from the postpartum human is provided, using a lateral flow device disclosed herein. In some embodiments, a method of selecting a postpartum human and detecting a level, or a presence or absence, of an analyte in the biological sample from the postpartum human is provided. The method includes, or consists of, (1) selecting a postpartum human about 0-3 months postpartum, (2) applying or contacting a biological sample (e.g., plasma or saliva or urine) obtained from the selected postpartum human with a lateral device disclosed herein (e.g., contacting the biological sample in the sample receiving region of a lateral flow device), and (3) detecting the level, or the presence or absence, of Flt-1 (e.g., sFlt-1) by measuring a detectable label in the indication region. In various embodiments, the postpartum human is about 1 week postpartum. In various embodiments, the postpartum human is about 2 weeks postpartum. In various embodiments, the postpartum human is about 3 weeks postpartum. In various embodiments, the postpartum human is about 4 weeks postpartum. In various embodiments, the postpartum human is about 1 month postpartum. In various embodiments, the postpartum human is about 2 months postpartum. In various embodiments, the postpartum human is about 3 months postpartum. In various embodiments, the postpartum human is about 0-2 weeks postpartum. In various embodiments, the postpartum human is about 2-4 weeks postpartum. In various embodiments, the postpartum human is about 1-2 month postpartum. In various embodiments, the postpartum human is about 3-4 month postpartum.

Diagnostic assays or methods of diagnosing preeclampsia or risk of preeclampsia, or its related disorders, in a pregnant female are provided using an immunochromatographic assay device (or lateral flow device). Various embodiments provide lateral flow devices for (optionally collection and) detection of the presence or absence of a measurable level of an analyte in a sample, the analyte comprising soluble fms-like tyrosine kinase 1 (sFlt-1), bound Flt-1, or both. The lateral flow device contains a sample receiving region, a development region, and an indication region comprising at a first location a first capture reagent or a modification capable of immobilizing the first capture reagent, wherein the regions each comprise a porous material and are in capillary contact with each other permitting a sample fluid to wick from the sample receiving region, through the development region, to the indication region; wherein the lateral flow device further comprises a first detection reagent comprising a detectable label and an antibody targeting Flt-1, the first detection reagent is capable of forming a complex binding an epitope of the analyte and being transported from the development region to the indication region, the first capture reagent is capable of forming a complex binding another epitope of the analyte.

In some embodiments, methods are provided for predicting a predisposition to preeclampsia or its related disorders in a subject. In some embodiments, methods are provided for diagnosing preeclampsia or its related disorders in a subject. In some embodiments, methods are provided for predicting the likelihood of recurrence of preeclampsia or its related disorders in a subject. In some embodiments, methods are provided for selecting a subject with preeclampsia or its related disorders for treatment. These methods include obtaining a sample of saliva or plasma from the subject, and an exemplary method includes contacting the sample with a lateral flow device, preferably the sample receiving region of the lateral flow device, and determining the presence or absence of a signal indicating the presence or absence of Flt-1, where a presence of a signal (i.e., positive detection) at the first location in the indication region of the lateral flow device predicts a predisposition to preeclampsia, diagnoses preeclampsia, predicts preeclampsia is likely to (re-)occur in the subject (e.g., within the next two weeks following the sample retrieval date) or the subject is selected for treatment of preeclampsia, and where an absence of the signal at the first location predicts that the subject does not have a predisposition to preeclampsia or preeclampsia, the subject is unlikely to have recurrence of preeclampsia, or the subject is not selected for treatment of preeclampsia. Exemplary preeclampsia-related disorders include but are not limited to eclampsia, idiopathic fetal growth restriction, or hemolysis, elevated liver enzymes, low platelet count (HELLP) syndrome.

In some embodiments of the above methods, a chase buffer is applied after applying the biological sample to the sample receiving region. In various embodiments, the chase buffer is applied about 15 seconds after applying the biological sample to the sample receiving region. In various embodiments, the chase buffer is applied about 30 seconds after applying the biological sample to the sample receiving region. In various embodiments, the chase buffer is applied about 45 seconds after applying the biological sample to the sample receiving region. In various embodiments, the chase buffer is applied about 60 seconds after applying the biological sample to the sample receiving region. In various embodiments, the chase buffer is applied about 15-60 seconds after applying the biological sample to the sample receiving region. In various embodiments, the chase buffer is applied about 30-60 seconds after applying the biological sample to the sample receiving region. In various embodiments, the chase buffer is applied about 30-90 seconds after applying the biological sample to the sample receiving region. In various embodiments, the chase buffer is applied about 45-90 seconds after applying the biological sample to the sample receiving region. In various embodiments, the chase buffer is applied within 120 seconds after applying the biological sample to the sample receiving region.

In various embodiments of the above methods, detecting the level, or the presence or absence, of the first detectable label is performed about 10 minutes after applying the biological sample to the sample receiving region. In various embodiments, detecting the level, or the presence or absence, of the first detectable label is performed about 15 minutes after applying the biological sample to the sample receiving region. In various embodiments, detecting the level, or the presence or absence, of the first detectable label is performed about 20 minutes after applying the biological sample to the sample receiving region. In various embodiments, detecting the level, or the presence or absence, of the first detectable label is performed about 30 minutes after applying the biological sample to the sample receiving region.

In various embodiments of the above methods, detecting the level, or the presence or absence, of the first detectable label is performed about 10-20 minutes after applying the biological sample to the sample receiving region. In various embodiments, detecting the level, or the presence or absence, of the first detectable label is performed about 10-30 minutes after applying the biological sample to the sample receiving region. In various embodiments, detecting the level, or the presence or absence, of the first detectable label is performed about 15-30 minutes after applying the biological sample to the sample receiving region. In various embodiments, detecting the level, or the presence or absence, of the first detectable label is performed about 15-45 minutes after applying the biological sample to the sample receiving region.

In various embodiments of the above methods, detecting the level, or the presence or absence, of the first detectable label is performed with a lateral flow reader. In various embodiments, detecting the level, or the presence or absence, of the first detectable label is performed with a densitometer.

In various embodiments of the above methods, detecting the level, or the presence or absence of the first detectable label is made according to any one of the cut-off concentrations or units described herein. In various embodiments, detecting the level, or the presence or absence of the first detectable label is made according to the color.

Additional embodiments provide the methods or assays are compatible with desktop or smartphone readers (e.g., Apps), configured with readily available camera and connectivity technology, and/or can be configured to work with multiple sample types. Results record and data sharing functionality are downloadable via an App. Preset algorithms and work-flows within Apps can be integrated with cameras and connectivity technology readily available in Smartphones. Some embodiments provide the methods or assays further include reading the result (e.g., presence, absence, or quantitation of Flt-1) on a desktop or smartphone by imaging the assay device.

In various embodiments of the above methods, a positive test (e.g., a detection of a level that is above a reference level, or a detection of the presence of the Flt-1 (soluble or membrane bound, and any and all isoforms) via the test, the subject (e.g., pregnant or postpartum human) will have a high likelihood of experiencing preeclampsia or a related condition within 1-4 weeks. In various embodiments, the subject (e.g., pregnant or postpartum human) will have a high likelihood of experiencing preeclampsia or a related condition within 1-2 weeks. In various embodiments, the subject (e.g., pregnant or postpartum human) will have a high likelihood of experiencing preeclampsia or a related condition within 2 weeks. In various embodiments, the subject (e.g., pregnant or postpartum human) will have a high likelihood of experiencing preeclampsia or a related condition within 2-3 weeks. As such interventions can be taken, as discussed herein.

Various embodiments provide the devices afford an “assay sensitivity” that is 100% or at least 99%, 98%, 96%, 95%, 94%, 93%, 92%, 91%, 90%, 85%, 80%, 75% or 70%. “Assay sensitivity” can be defined as the percentage of true positive incidence over a total incidence of true positive and false negative; for example, in detecting Flt-1. Various embodiments provide the devices afford an “assay specificity” that is 100% or at least 99%, 98%, 96%, 95%, 94%, 93%, 92%, 91%, 90%, 85%, 80%, 75%, 70%, 65%, 60% or 50%. “Assay specificity” can be defined as the percentage of true negative incidence over a total incidence of false positive and true negative. Various embodiments provide the devices afford an assay positive predictive value that is 100% or at least 99%, 98%, 96%, 95%, 94%, 93%, 92%, 91%, 90%, 85%, 80%, 75%, 70%, 65%, 60% or 50%. Assay positive predictive value can be defined as the percentage of true positive incidence over a total incidence of true positive and false positive. Various embodiments provide the devices afford an assay negative predictive value that is 100% or at least 99%, 98%, 96%, 95%, 94%, 93%, 92%, 91%, 90%, 85%, 80%, 75%, 70%, 65%, 60% or 50%. Assay negative predictive value can be defined as the percentage of true negative incidence over a total incidence of true negative and false negative.

In one embodiment, the devices and the methods of detecting Flt-1 using the devices provide an assay sensitivity of 100% and a negative predictive valuation of 100%. In another embodiment, the devices and the methods of detecting Flt-1 using the devices provide an assay sensitivity of 99% and a negative predictive valuation of 99%. In another embodiment, the devices and the methods of detecting Flt-1 using the devices provide an assay sensitivity of 98% and a negative predictive valuation of 98%. In another embodiment, the devices and the methods of detecting Flt-1 using the devices provide an assay sensitivity of 97% and a negative predictive valuation of 97%. In another embodiment, the devices and the methods of detecting Flt-1 using the devices provide an assay sensitivity of 96% and a negative predictive valuation of 96%. In another embodiment, the devices and the methods of detecting Flt-1 using the devices provide an assay sensitivity of 95% and a negative predictive valuation of 95%.

In some embodiments, a method of performing a lateral flow assay using the lateral flow device in the form a strip comprises dropping, inserting or adding the strip to a (vertically placed) borosilicate glass tube containing about 15 μL of a biological sample (e.g., plasma), so as to allow contact of the strip with the biological sample for at least about 30 seconds, and subsequently adding about 100 of a chase buffer to the bottom of the borosilicate glass tube, allowing for the chase buffer to contact the strip and allowing time of at least 14 minutes or about 14.5 minutes for lateral flow in the strip, and followed by reading result of the strip (e.g., detecting presence or amount of the detectable label at the first or the second location of the indication region, by using a densitometer or by visual grading.)

### Treatment Methods

Various embodiments provide methods for administering a therapy for treating and/or managing preeclampsia. Various embodiments provide methods for administering a therapy for treating a preeclampsia-related disorder to a pregnant human in need thereof. Various embodiments provide methods for administering a therapy for treating a preeclampsia-related disorder to a postpartum human in need thereof. The methods comprise detecting an amount of Flt-1 in a biological sample obtained from the patient using a lateral flow device described herein, and administering a dosage amount of the therapy to the pregnant human or to the postpartum human.

Some embodiments provide methods for administering a therapy for treating and/or managing preeclampsia or a preeclampsia-related disorder to a pregnant human in need thereof, which comprise detecting an amount of Flt-1 (e.g., sFlt-1) in a biological sample obtained from the patient above a reference level with a lateral flow device described herein, and administering a dosage amount of the therapy to the pregnant human detected with the amount of Flt-1 (e.g., sFlt-1) above the reference level.

Further embodiments provide methods for administering a therapy for treating and/or managing preeclampsia or a preeclampsia-related disorder to a pregnant human in need thereof, which include administering a dosage amount of the therapy to the pregnant human detected with an amount of Flt-1 (e.g., sFlt-1) in a biological sample obtained from the pregnant human above a reference level with a lateral flow device described herein.

Additional embodiments provide methods for administering a therapy for treating and/or managing preeclampsia or a preeclampsia-related disorder to a pregnant human in need thereof, which include requesting results of an assay using a lateral flow device disclosed herein of the pregnant human's level of Flt-1 in a biological sample obtained from the pregnant human, and administering a dosage amount of the therapy to the pregnant human based on the level of Flt-1 in the biological sample. In some aspects, a positive detection in the lateral flow device, or a level of Flt-1 above a reference value, indicates that pre-eclampsia is likely to occur in the pregnant human in the following two weeks. In some aspects, a positive detection in the lateral flow device, or a level of Flt-1 above a reference value, indicates that pre-eclampsia is likely to occur in the pregnant human in the following week. In some aspects, a positive detection in the lateral flow device, or a level of Flt-1 above a reference value, indicates that pre-eclampsia is likely to occur in the pregnant human in the following three weeks. As such, intervention in the form of therapies and/or heighten observations (e.g., as compared to one who does not have pre-eclampsia) or monitoring can be administered or provided to the subject.

Exemplary therapies suitable for treatment and/or management of preeclampsia or related disorders include but are not limited to an anti-Flt-1 antibody or fragment thereof, a steroid treatment (which helps with improvement of prematurity related lung immaturity), or magnesium sulfate (which helps with seizure prophylaxis). Anti-Flt-1 antibodies, e.g. anti-sFlt-1 antibodies, are described in U.S. Pat. No. 9,592,331, which is herein incorporated by reference in its entirety.

In other embodiments, a patient detected with an amount of Flt-1 above a reference level, by using a lateral device described herein, is referred to a hospital for appropriate care of premature fetus (e.g., a tertiary care hospital with level IV or III neonatal intensive care unit, MCU).

In further embodiments, the methods include performing one or more apheresis treatments to a patient detected with an amount of Flt-1 above a reference level. In one example, the apheresis treatment comprises dextran sulfate apheresis, which lowers circulating sFlt-1. In various aspects, the apheresis treatment is an extracorporeal apheresis with one or more dextransulfate cellulose columns.

### Systems

Various embodiments of the present invention provide for a system comprising a lateral flow device of the present invention and a densitometer. Examples of densitometers include but are not limited to those made by METTLER TOLEDO, ANTON PAAR, AXXIN, X-RITE, TECHKON.

Various embodiments of the present invention provide for a system comprising a lateral flow device of the present invention and a lateral flow reader. Examples of lateral flow readers include but are not limited to those made by DRUMMON SCIENTIFIC, AXXIN, GENPRIME, and HAMAMATSU.

In various embodiments, these systems may further comprise buffers such a chase buffer.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example A

A sample panel of 92 plasma samples were tested in-house with the currently assay. Results obtained from the large screen showed acceptable performance providing a positive predictive value of nearly 80%, a negative predictive value of 100%, specificity at 69%, and sensitivity at 100% based on a concentration cutoff of 7.25 ng/mL.

A bulk production of LFAs was prepared to match the performance of the previously mentioned 92 sample panel and would be used to test a panel of 295 plasma samples. The 295 plasma samples were tested in duplicate and visual scoring was performed by two different users per each test strip, followed by reading on an Axxin AX-2X-S Lateral Flow Instrument. Compiled test results show performance providing a positive predictive value of 70%, a negative predictive value of 100%, specificity at 80%, and sensitivity at 100% based on a concentration cutoff of 7.5 ng/mL.

Reagents for producing pre-eclampsia test strips for detection of sFlt-1 in plasma samples:

TABLE 1
Reagents for producing pre-eclampsia
test strips for detection of sFlt-1.

| | Concentration/Percentage | |
|---|---|---|
| Test Line Components | | |
| Polystreptavidin | 0.1 mg/mL-0.2 mg/mL | |
| TRIS pH 8 | 1.6 | mM |
| Sucrose | 2% | |
| Control Line Components | | |
| Goat anti-Mouse Antibody | 0.3 | mg/mL |
| 40 mM TRIS pH 8, 50% Sucrose | 0.8 mM and 1% | |
| Sample Pad Block Solution | | |
| Pluronic F127 | 0.5% | |
| MgCl₂ | 1M | |
| Latex Conjugate | | |
| 400 nm Red Carboxylated Latex | 10 | mg |
| DuoSet Capture Antibody | 0.5 | mg |
| 0.1M MES Buffer pH 6.0 | 0.1M | |
| 0.1M Borate Buffer pH 8.6 | 0.1M | |
| EDAC | 15 | mg/mL |
| NHS | 50 | mg/mL |
| 1XPBS | 1X | |
| 50 mM Borate with 6% Casein (7 Day | 50 mM and 1% | |
| Cured) | | |
| Latex Storage Buffer | | |
| 0.1M Borate pH 8.6 | 50 | mM |
| 50 mM Borate with 6% Casein (7 Day | 50 mM and 1% | |
| Cured) | | |
| Final Conjugate Solution | | |
| mAb anti-VEGF R1 DuoSet Capture | 0.1% | |
| conjugated to 400 nm Red Latex | | |
| Sucrose | 10% | |
| Trehalose | 2% | |
| Latex Storage Buffer | QS to Final Volume | |
| Biotinylated Antibody Solution | | |
| DuoSet Detection Antibody | 22.5 | μg/mL |
| Sucrose | 10% | |
| Trehalose | 2% | |
| Colored Dye | Trace Amount | |
| Biotinylated Antibody Diluent | QS to final volume | |
| Biotinylated Antibody Diluent | | |
| BSA | 1% | |
| 1XPBS | QS to final volume | |
| Chase Buffer | | |
| Pluronic F127 | 0.75% | |
| 1XPBS | QS to final volume | |
| 1M TRIS pH 8 | | |
| TRIS HCl | 1M | |
| NaOH | 10N | |
| diH₂O | QS to final Volume | |
| 40 mM TRIS with 50% Sucrose | | |
| Buffer pH 8.0 ± 0.1 | | |
| Sucrose | 50% | |
| 1M TRIS pH 8 | 40 | mM |

| | |
|---|---|
| diH₂O, 18.2 mΩ | QS to final volume |

Assembly:

1. Strip
Polystreptavidin test line striped at 0.1 mg/ml-0.2 mg/mL in 1×PBS, 1.6 mM TRIS pH 8, 2% Sucrose solution at 47 mm from the bottom edge of the strip.
Goat anti-Mouse control line 0.3 mg/ml in 1×PBS, 0.8 mM TRIS pH 8, 1% Sucrose solution at the 52 mm from bottom edge of the strip.

2. Conjugate
DuoSet Capture Antibody is conjugated to 400 nm Red Latex particles at a mass ratio of 40:1 (Beads:Ab)
Latex conjugates are prepared in 50 mM Borate, 1% Casein, 10% Sucrose, 2% Trehalose, pH 8.5 to a final Latex solids concentration of 0.1% by diluting with conjugate diluent.

3. Sample Pad Buffer/Block
Add appropriate amount of anhydrous MgCl₂ solid into 10% stock of Pluronic F127 solution to reach a final 1M MgCl₂ concentration.
Pipette buffer onto precut sample pad material that was organized and separated on a screen. Approximately 2.5 mL was required for each 300 mm×22 mm pad. After pipetting the appropriate amount of buffer onto each pad and allowing the pad to soak, the screen was placed in a 40° C. forced air oven for 1 hour.

4. Test Procedure
Pipetted 15 μL of plasma sample into a borosilicate glass tube.
Drop the test strip into the glass tube so that the sample pad is in direct contact with the plasma sample.
Allowed for 30 seconds to allow sample to absorb into the strip before pipetting 100 μL of chase into the bottom of each tube.
Allowed strip to run for 14.5 additional minutes.
Read the result by assigning a visual grade based on the DCN Visual Grade Chart.

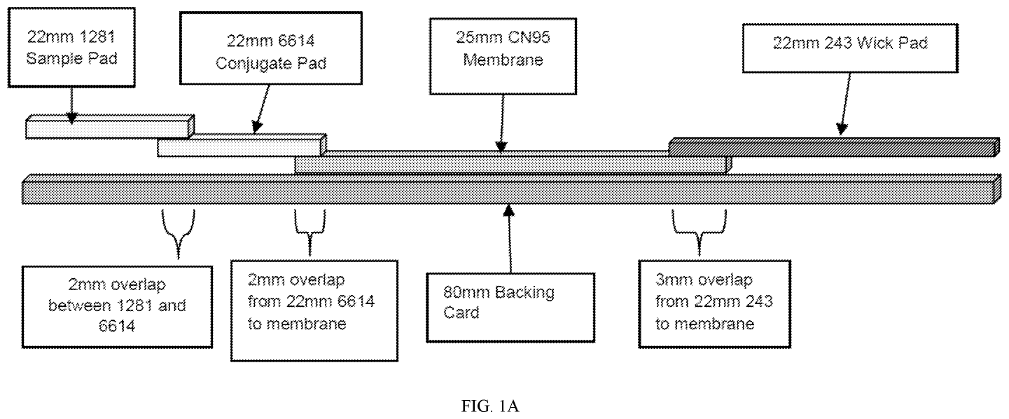

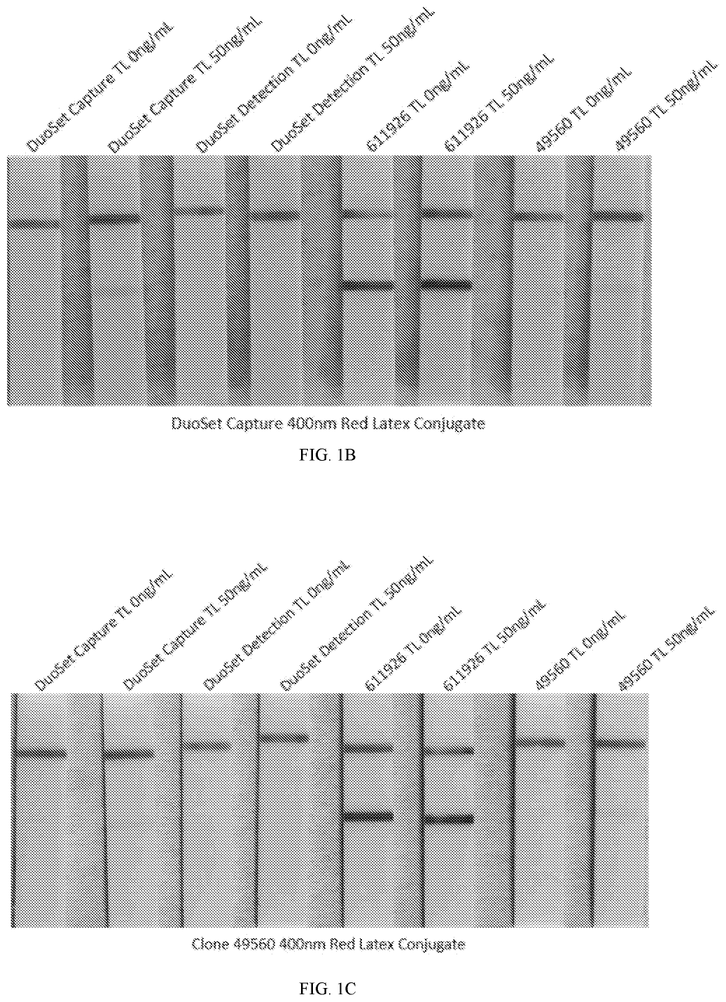

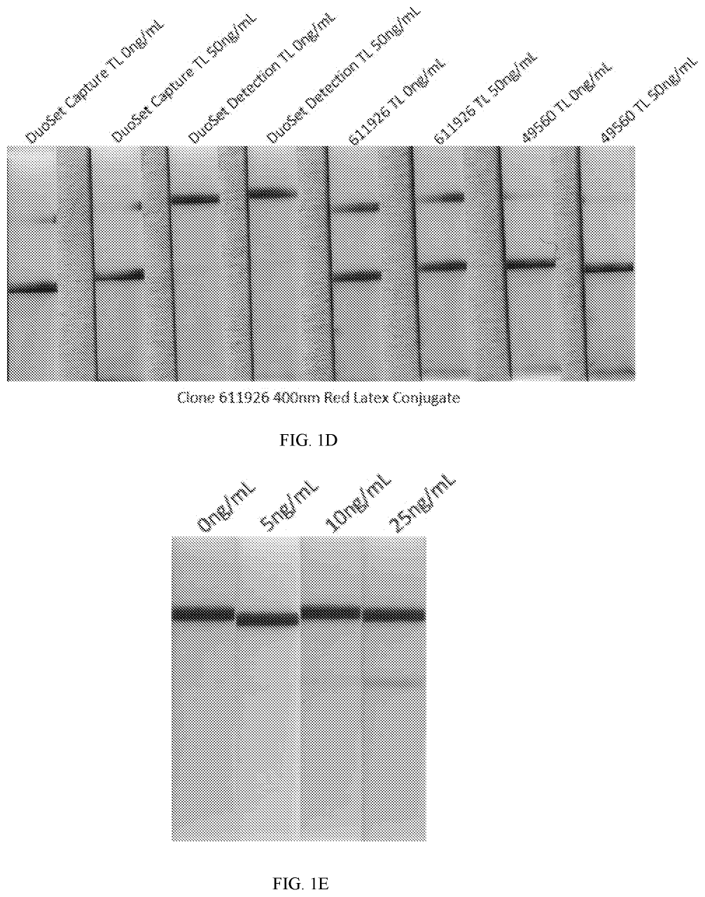

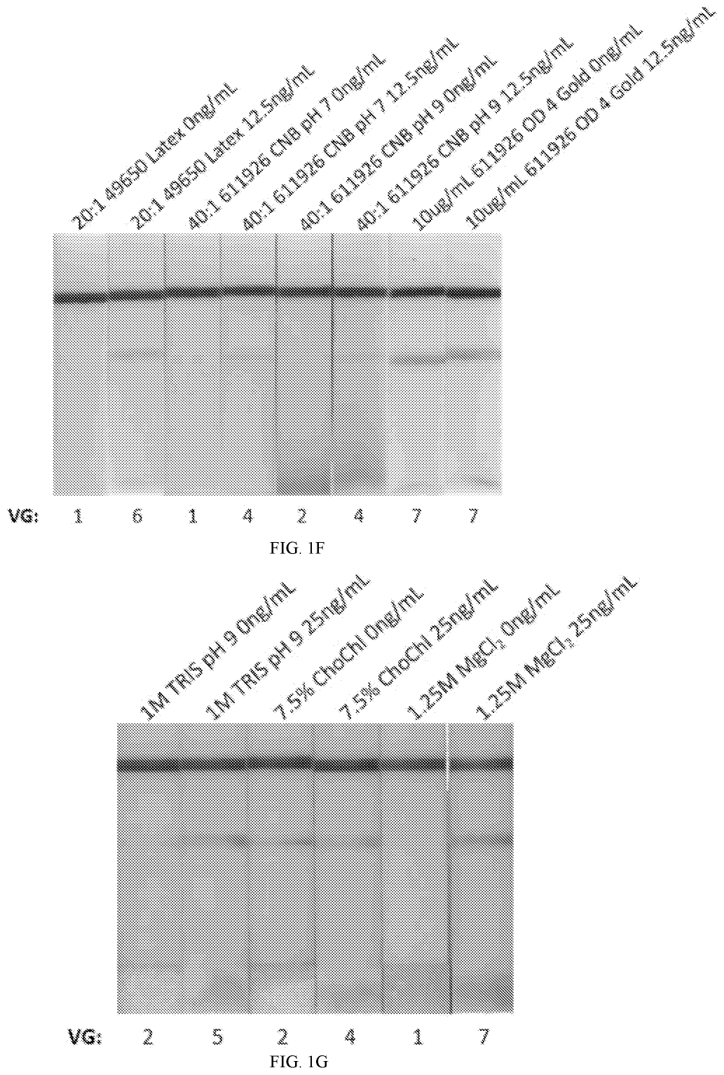

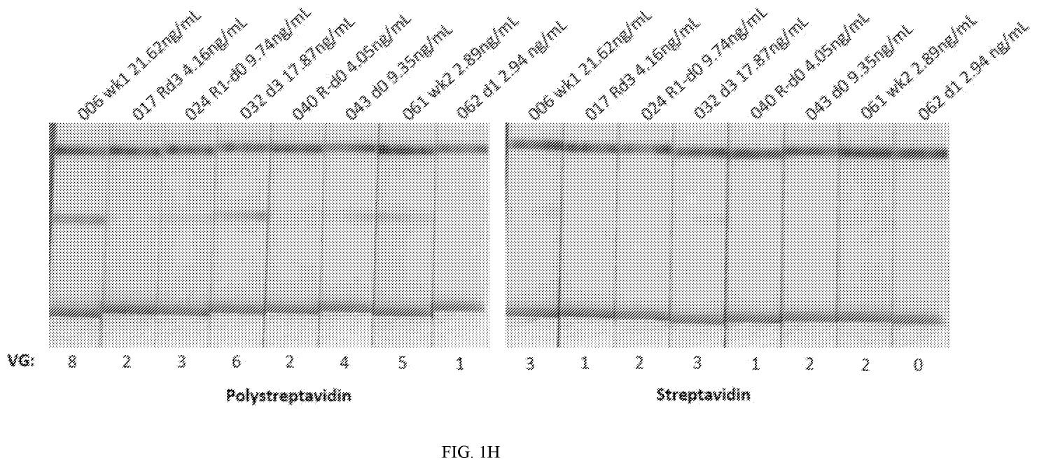

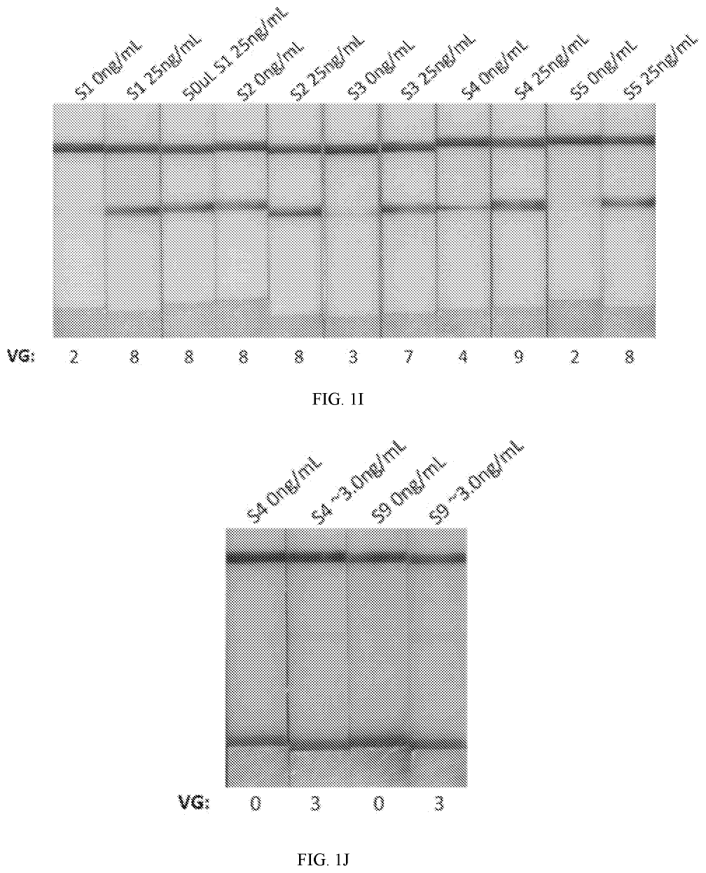

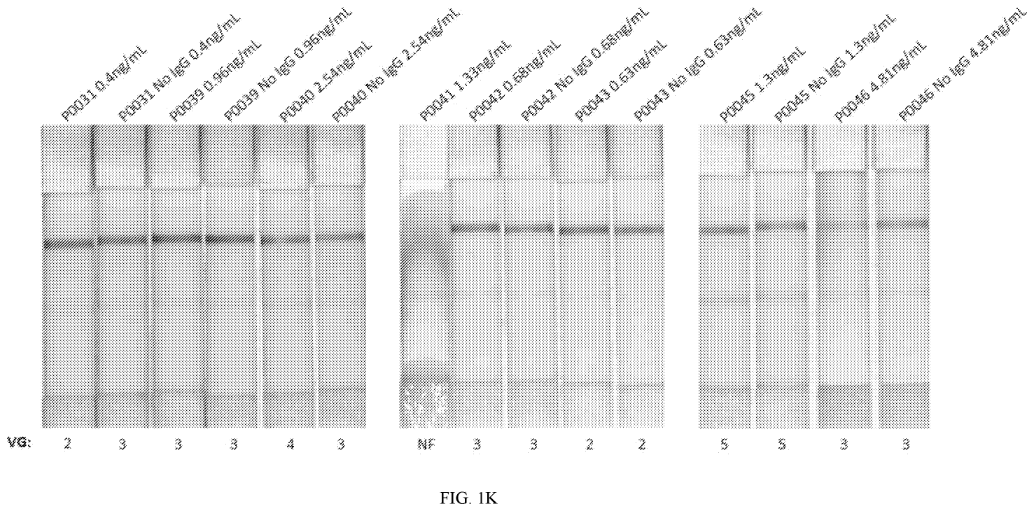

A lateral flow strip layout is depicted in a diagram in FIG. 1A (not to scale).

TABLE 2
Components and details in an exemplary lateral flow strip as depicted in FIG. 1A.

| | | | Location from | | |
|---|---|---|---|---|---|
| | | Length | bottom of Strip | | |
| Part | Material | (mm) | (mm) | Thickness | |
| Membrane | Sartorius CN95 | 25 mm | 37 mm | 240-270 | μm |
| Conjugate Pad | Ahlstrom 6614 | 22 mm | 18 mm | 420 | μm |
| Sample Pad | Ahlstrom 1281 | 22 mm | 0 mm | 380 | μm |
| Wick Pad | Ahlstrom 243 | 22 mm | 58 mm | 950 | μm |
| Backing Card | Lohmann | 80 mm | N/A | 500 | μm |

| | |
|---|---|
| Location of | 10 mm from bottom edge of Nitrocellulose membrane. 47 mm from |
| Test Line | the bottom of strip (referenced from the conjugate pad end) |
| Location of | 15 mm from bottom edge of Nitrocellulose membrane. 52 mm from |
| Control Line | bottom of strip (referenced from the conjugate pad end). |
| Overall Strip width | 4 mm |

### Procedures

I. Blocking 1281 with Pluronic F127 and MgCl2

TABLE 3
Materials and equipment.
Description

Anhydrous MgCl₂ PN: M8266
Pluronic F127
Millipore 0.2 um Filter PN: 565-0020
Ahlstrom Grade 1281
Forced Air Oven Asset No. OV003

1. Generated 40 mL of 10% Pluronic solution. Combined 4 grams of Pluronic F127 powder with 30 mL of diH₂O. Mixed until the Pluronic had fully dissolved. QS′d solution to 40 mL with diH₂O.
2. Prepared buffer solution. Combined 9.52 g of anhydrous MgCl₂ with 70 mL of diH₂O in a 250 mL glass beaker. Mixed until the powder had formed a uniform phase solution. Added 10 mL of 10% pluronic F127 solution to the mixture. Mixed briefly than QS′d to 100 mL with diH₂O with a graduated cylinder. Returned to beaker and mixed until solution was completely uniform and MgCl₂ was completely dissolved.
3. Filtered with Millipore 0.2 um Filter via vacuum.
4. Placed twenty 22 mm×300 mm cut 1281 pads onto a screen.
5. Pipetted 2 mL of prepared buffer onto the 22 mm cut sample pad saturating the entire pad evenly.
6. Placed pads into a 40° C. forced air oven immediately after blocking and dried for ≥60 min.
7. Stored dried pads in desiccated bin, sealed in foil pouch with desiccant.
II. 2 mL hVEGF R1 Antibody Latex Conjugation

TABLE 4
Materials and equipment.
Description

Magsphere 0.4 um Red Latex PN: CAR400NM
0.1M MES, pH 6.00
EDC, PN: 22981
NHS, PN: 130672
1X PBS, PN: 10023
Anti-hVEGF Clone DuoSet Capture, 3.85 mg/mL
6% casein in 50 mM borate (7 day-cured), pH 8.59
Storage Buffer
Vortex
Beckman Coulter Spec, DU730, SP002
Thermo Scientific Centrifuge, Legend XTR, CE004

### Method:

### Preparation and Washes

### Vb=2000 uL

1. Sonicated stock Magsphere 10% Red latex solution 10 seconds at 25% amplitude.
2. In two clean 1.7 mL tube, added 100 uL of Magsphere 10% 400 nm Red latex particle solution into 900 uL of 0.1M IVIES pH 6.0.
3. Centrifuged for 15 minutes at 17,000×g at 8° C.
4. Pulled off supernatant and replaced with 1 mL of 0.1 M IVIES.
5. Sonicated for 30-45 seconds at 25% amplitude.
6. Repeated step 3. Added 0.8*Vb uL of 0.1M IVIES pH 6. Sonicated at 25% power for 30-45 seconds.

### Activation

7. Prepared fresh 15 mg/mL EDC and 50 mg/mL NHS in 0.1M IVIES pH 6:
a) Weighed out NHS and EDC in clean 1.7 mL tubes and seal top until ready to use:
i. Weighed out 15 mg EDC.
ii. Weighed out 50 mg NHS.
   8. Added 1 mL 0.1M IVIES to EDC and NHS (see #8 for calculated volumes) and vortexed to mix. Note: once the IVIES is added, the solutions must be used within 5 minutes.
   9. Added 0.01*Vb (10 uL for 1000 uL) of EDC followed by 0.2*Vb (200 uL for 1000 uL) of NHS to the particles then vortexed to mix.
   10. Continued mixing with an orbital shaker for 30 minutes at R.T. to activate.

### Conjugation

11. Centrifuged particles for 10 minutes at 15,000×g at 8° C.
12. Removed as much supernatant as possible without disturbing the pellet and replaced with 1 mL of 1×PBS.
13. Sonicated 30-45 seconds at 25% amplitude.
14. Centrifuged particles for 10 minutes at 15,000×g at 8° C.
15. During step 14 prepared protein for conjugation:
a) 40:1 conjugation: needed 250 ug protein (per conjugation) for 1000 uL 1% particle batch.
b) Antibody Clone DuoSet Capture concentration 3.85 mg/mL so 65 uL used for 40:1.
16. Removed as much supernatant as possible without disturbing the pellet. Added 950 uL of 1×PBS.
17. Sonicated 30-45 seconds at 25% power.
18. Added antibody as calculated in step 15 to designated particle aliquot.
19. Vortexed immediately. Mixed for 3 hours with an orbital shaker at RT.

### Quenching (Overnight)

20. Added 0.2*Vb of 6% Casein then vortexed and then continued to mix with an orbital shaker for ≥12 hours at RT.

### Washing and Quantification

21. Centrifuged particles for 10 minutes at 15,000×g at 8° C.
22. Removed as much supernatant as possible without disturbing the pellet and replaced with 1 mL of storage buffer.
23. Sonicated 30-45 seconds at 25% amplitude.
24. Centrifuged particles for 10 minutes at 15,000×g at 8° C.
25. Removed as much supernatant as possible without disturbing the pellet and replaced with 1 mL of wash/storage buffer.
26. Sonicated 30-45 seconds at 25% amplitude.
27. Repeated step 25-27 but added 0.8*Vb before resuspending.
28. Quantified yield with the spectrophotometer:

### Red Latex:

1. Blanked spec with water at A560 nm.
2. Made a 200× dilution of stock 10% particles by: adding 5 uL to 995 uL of water, vortexed after addition, then took 100 uL of this stock into 900 uL of water, vortexed, read A560 nm.
3. Added 5 uL of fresh conjugate to 995 uL (1:200) of DI water, mix, then read A560 nm.
4. Store conjugate at 2-8° C.

TABLE 5
Quantification: final red latex conjugate concentrations.

| | A560 | Stock |
|---|---|---|
| Conjugate | nm | Concentration |
| 0.4 um Magsphere 5%, 0.005% (1:200 dilution) | 0.892 | 1% (from |
| | | manufacturer) |
| 0.4 um Magsphere Clone DuoSet Capture Ab | 0.962 | 1.08% |
| 40:1, 0.005% (1:200 dilution) Tube 1 | | |
| 0.4 um Magsphere Clone DuoSet Capture Ab | 0.925 | 1.04% |
| 40:1, 0.005% (1:200 dilution) Tube 2 | | |
| 0.4 um Magsphere Clone DuoSet Capture Ab | 1.01 | 1.13% |
| 40:1, 0.005% (1:200 dilution) Combined | | |

| | Concentration |
|---|---|
| Conjugate | (% solids) |
| 0.4 um Magsphere Clone DuoSet Capture Ab 40:1 | 1.13% |

### III. Spraying Biotinylated Antibody and Spraying Latex for Optimal Rate Determination

TABLE 6
Materials and Equipment.
Description

0.1% 40:1 400 nm Latex DuoSet Capture Conjugate w/10% Sucrose
2% Trehalose
Free Biotinylated DuoSet Detection Antibody, 22.5 ug/mL w/10% Sucrose
22 mm Ahlstrom Grade 6614
Biodot Striper XZ3050 Asset No. SR004
Forced Air Oven Asset No. OV003

### Method:

### Spray Conjugate

1. Vortexed stock latex conjugate.
2. Cleaned Airjet with 10 cycles of Bioterge and 10 cycles of DiH₂O
3. Sprayed latex conjugate using the BioDot Airjet on each pad material using the following parameters aligning the center of the spray at ˜17 mm from the bottom edge of the pad.
PSI=6 (while spraying)
Speed 25 mm/s
Acc=1000 mm/s\hat{ }2
Z down=50
Y=53
Rate=8 uL/cm, 10.5 μL/cm, 12 uL/cm
   4. Placed 22 mm pad at the very bottom of the platform and placed Airjet aligned with red mark on support arm
   5. Sprayed then dried for ˜0.5 hour at 40° C.

### Spray Biotinylated Ab

6. Cleaned Airjet with 10 cycles of Bioterge and 10 cycles of DiH₂O
7. Sprayed antibody conjugate using the BioDot Airjet on each pad material using the following parameters aligning the center of the spray at ˜5 mm from the bottom edge of the pad.
PSI=6 (while spraying)
Speed 25 mm/s
Acc=1000 mm/s\hat{ }2
Z down=50
Y=45
Rate=6 μL/cm
   8. Placed 22 mm pad with conjugate at the very bottom of the platform and placed Airjet aligned with red mark on support arm
   9. Sprayed then dried for 1 hour at 40° C.
   10. Store sprayed pads in a desiccated pouch at room temperature.

### IV. Striping Polystreptavidin on CN95

TABLE 7
Materials and Equipment.
Description

CN95
1XPBS
Polystreptavidin, 3.89 mg/mL
Coat Anti Mouse, 10 mg/mL
40 mM TRIS pH 8 50% Sucrose
Biodot Striper XZ3050 Asset No. SR004
Force Air Oven Asset No. OV003

### 1. Prepared Antibody Solutions.

Combined 25.5 uL of stock polystreptavidin solution at 3.89 mg/mL with 40 uL of 40 mM TRIS pH 8 50% sucrose solution and 934.5 uL of 1×PBS. Mixed via vortex.

Combined 30 uL of stock goat anti mouse antibody solution at 10 mg/mL with 20 uL of 40 mM TRIS pH 8 50% sucrose solution and 950 uL of 1×PBS. Mixed via vortex.

2. Striped antibodies at 10 mm TL and 15 mm C_{L} using the reverse priming method
Washed out frontlines with 10 cycles of 0.05% bioterge and 10 cycles of diH₂O Set frontline dispenser to the following parameters: Rate=1.0 ul/cm, Length=300 mm, Speed=35 mm/s, ACC=1000 m/s, Z down=49
Striped membrane
Placed membranes at 40° C. immediately after striping and dried for 30 min.
When finished, cleaned frontlines with 10 cycles of 0.05% bioterge and 10 cycles of dH₂O
Stored striped/dried membranes in a desiccated bin, sealed in a foil pouch with desiccant.

### V. Data Analysis

Visual grading of the signal level for the qualitative assay was performed the DCN 0-10 Grading Scale (FIG. 11). The DCN grading scale has been generated and used internally by DCN for standardization of subjective strip reading among multiple users.

Supplemental scoring was obtained later in the development project by using an Axxin AX-2X-S Lateral Flow Instrument. Testing was performed using the default test type designated ‘Carrier Blue Latex’. Line positions were adjusted using the acquire feature and then set under the design tab. A drawer and strip holder specifically designed for the Axxin instrument, provided by the company, were used to hold the currently designed LFA in place for the read. The test line peak height data was used for analysis.

VII.i. Results—Antibody Screening

Four antibodies were received for evaluation, 2 of which were part of an ELISA kit. Due to the low quantity of the antibodies, screening of the reagents could be carried out with only a single particle type. A latex particle was chosen due to the lower amount of antibody required to prepare the conjugate.

DuoSet Capture Latex Conjugate Initial Half Strip Test (FIG. 1B): Results were obtained from half strips run for 15 minutes with 15 uL of spiked and unspiked buffer and 5 uL of 0.1% latex conjugate solution. All strips were striped with 1 mg/mL test line concentration (DuoSet Detection TL contained a high concentration of BSA which likely reduced the amount of antibody bound to the membrane). As shown in FIG. 1B, results show separation for DuoSet Capture and Clone 49560 antibodies between the unspiked and 50 ng/ml antigen spiked buffer solution. This suggested that the self-pairing of the DuoSet Capture as both test line and conjugate antibody as well as the DuoSet Capture as the conjugate antibody and Clone 49560 on the test line show potential to be candidates for use in the lateral flow devices.

Clone 49560 Latex Conjugate Initial Half Strip Test (FIG. 1C): Results were obtained from half strips run for 15 minutes with 15 uL of spiked and unspiked buffer and 5 uL of 0.1% latex conjugate solution. All strips were striped with 1 mg/mL test line concentration (DuoSet Detection TL contained a high concentration of BSA which likely reduced the amount of antibody bound to the membrane). As shown in FIG. 1C, results show separation for DuoSet Capture and Clone 49560 antibodies between the unspiked and 50 ng/ml antigen spiked buffer solution. This suggested that the self-pairing of Clone 49560 as both test line and conjugate antibody as well as Clone 49560 as the conjugate antibody and the DuoSet Capture antibody on the test line show potential to be candidates for use in the lateral flow devices.

Clone 611926 Latex Conjugate Initial Half Strip Test (FIG. 1D): Results were obtained from half strips run for 15 minutes with 15 uL of spiked and unspiked buffer and 5 uL of 0.1% latex conjugate solution. All strips were striped with 1 mg/mL test line concentration (DuoSet Detection TL contained a high concentration of BSA which likely reduced the amount of antibody bound to the membrane). Unlike the other two conjugates/conjugate antibodies Clone 611926 did not show distinguishable separation between unspiked and 50 ng/ml antigen spiked buffer solution. The reasoning could be a number of different items including the label, the antibody specificity, conjugation method, and other optimization steps such as salt concentration.

Initial results showed that 2 of the antibodies (DuoSet ‘Capture’ and Clone 49560) each functioned well self-paired as well as in both capture and detector roles with each other in a total of 4 combinations. These combinations showed results where 0 ng/mL produced no visible test line and the 50 ng/mL level was visible. Test line visual grades from testing 50 ng/mL in these combinations ranged from a 3-5. It is currently showing that the reagents are functional and can be used for further development.

The DuoSet ‘Detection’ antibody came lyophilized in BSA and therefore was difficult to evaluate as both the TL reagent and conjugated to a particle. This antibody was striped and showed low intensity TL signals with the 50 ng/ml antigen concentration when tested with 49560 and DuoSet ‘Capture’ conjugates. This is likely due to the relatively low proportion of the antibody compared to total protein on the test line and is therefore not necessarily a good measure of performance. This antibody was not conjugated to a particle due to this high concentration of storage protein in the solution. Given the small amount of antibody available, purification was not carried out. Because this antibody comes biotinylated, it can be evaluated as the detector by combining with streptavidin latex particles and also as free biotinylated antibody to be captured by a streptavidin test line.

The 611926 antibody conjugated to latex or striped as the test line showed strong NSB when self-paired, and when paired with DuoSet ‘Capture’ and 49560 antibodies, regardless of orientation. It did not show NSB when paired with DuoSet ‘Detection’ antibody TL but that is likely due to low antibody concentration of the TL.

VII.ii. Results—Exploring the Biotin-Avidin Configuration

Examine if a biotin-avidin test system could be used given that the DuoSet Detection antibody arrived pre-biotinylated. This system uses a polystreptavidin test line in combination with a conjugated antibody and free biotinylated antibody to generate a sandwich that then binds to the polystreptavidin test line due to the strong affinity between biotin and avidin. DuoSet Capture and Clone 49560 latex conjugates were selected for initial testing with this system due to their performance in previous testing.

Biotin-Avidin Test System Dose Response, ‘Capture’ Ab Latex Conjugate: Results were obtained from half strips run for 15 minutes with 15 uL of unspiked and spiked buffer (recombinant antigen), 5 uL of 0.1% DuoSet ‘Capture’ latex conjugate solution, and ˜124 ng of Biotinylated Detection antibody. All strips were striped with 1 mg/mL polystreptavidin test line concentration. As shown in FIG. 1E, results demonstrate a visually observable dose response ranging from 5 ng/mL up to 25 ng/mL and a determinable difference between the 0 ng/mL sample and 5 ng/mL suggesting good specificity.

After a number of experiments to explore addition order and biotinylated antibody amounts, a rough set up was determined that showed significant promise to increase initial assay performance from the standard capture assay using the Biotin-Avidin system.

VII.iii. Results—Exploring CNBs and Gold

Initial testing was performed only with the red latex label, with initial feasibility being operable, two other labels were explored: colloidal gold and cellulose nanobeads to determine if improvements to the performance of the assay could be achieved and to examine if the antibody clone 611926, which had been characterized as having desirable reactivity to sFlt-1, could work with a different label.

Testing Clone 611926 CNB and Gold Conjugates with Buffer (FIG. 1F): Results were obtained from half strips run for 15 minutes with 15 uL of spiked and unspiked buffer, 1.38 uL of 90 ng/mL Biotinylated DuoSet Detection antibody. All strips were striped on CN95 membrane with polystreptavidin at a concentration of 1 mg/mL at the test line. This experiment was performed to compare the currently selected system using latex nanoparticles with the 611926 antibody labeled on two labels not screened in the original testing: CNBs and colloidal gold.

While clone 611926 did show some promise when bound to the CNB's (colloidal gold did not show separation) it was determined that the latex label in combination with the biotin-avidin system showed the better result at the time. CNB's were also examined with the DuoSet Capture and 49650 antibodies but again more promise appeared with the latex label and therefore pursuit of a different visual label was abandoned.

VII.iv. Results—Optimization with the Plasma Matrix

After focus had shifted to a single label, antibody pairing, and test system, further embodiments using plasma matrix was explored in depth by testing antibody loading, assay configuration, sample pads, test line concentration, and sample additives such as surfactants, salts, and proteins.

Salt Testing with 40:1 Latex Conjugate and ‘Good’ Plasma (FIG. 1G): Results were obtained from full strips run for 10 minutes with 15 uL of 25 ng/ml antigen spiked and unspiked pooled plasma (pooled all samples that have demonstrated differentiation between negative and positive), 1.38 uL of 90 ng/mL Biotinylated DuoSet Detection antibody, and 5 uL of 0.1% DuoSet Capture latex conjugate solution per strip. CN95 membrane. All strips were striped with polystreptavidin at a concentration of 0.75 mg/mL at the test line. Due to increased NSB observed when switching from a spiked buffer sample to a spiked plasma sample (which showed variation depending on the plasma donor), a number or additives were testing in an attempt to eliminate the NSB without eliminating the specific binding; this image shows the most potent additives for elimination of NSB.

Comparing Polystreptavidin and Streptavidin at 30 Minute Development Time (FIG. 1H): Results were obtained from full strips run for 30 minutes with 15 uL of characterized unaltered plasma samples. CN95 membrane. 12.5 uL/cm 0.1% DuoSet Capture Latex. 8 uL/cm 22.5 ng/mL DuoSet Detection Solution. 0.1 mg/mL Polystreptavidin or 2 mg/mL Streptavidin test line concentration. Results demonstrate the significant performance difference between polystreptavidin and streptavidin with regards to sensitivity. Polystreptavidin shows significantly stronger TL intensity.

FIG. 18 shows visual grade data plotted against sFlt concentration. Besides the 2889 pg/mL sample, the remaining concentrations show increasing TL intensity with increasing sFLT-1 concentration.

Testing showed a number of possibilities for mild performance improvement with plasma sample testing; however, the most significant improvement appeared with the addition of salt, most specifically MgCl₂. A second main focus of development occurred after the discovery of MgCl₂ and that was the exploration of the test line reagent and concentration. Original biotin-avidin system testing was all performed with polystreptavidin; however, after moving to the plasma matrix it appeared that polystreptavidin was simply too sensitive and in some cases generated a significant amount of nonspecific binding. After exploring streptavidin and neutravidin at a number of concentrations and development times, it started to become evident that a significant reduction of the polystreptavidin concentration provided the best option for assay performance improvement.

VII.v. Results—Testing a Plasma Sample Panel of 92 Samples

Testing with spiked plasma and a limited number of characterized clinical plasma sample appeared to show acceptable performance. Testing with a larger sample set was carried out to better understand the performance of the assay. 92 samples were characterized and sent to DCN by the client to be tested in house.

FIG. 19 shows visual grade data from testing the 92 characterized samples in house.

TABLE 8
Sensitivity and Specificity.

| | |
|---|---|
| Total negative samples tested (less than 7.25 ng/ml) | 42 |
| Total positive samples tested (greater than 7.25 ng/ml) | 50 |
| True negatives | 29 |
| True positives | 50 |
| False negatives | 0 |
| False positives | 13 |
| Sensitivity (TP/(TP + FN)) | 100.0% |
| Specificity (TN/(FP + TN)) | 69.0% |
| Positive predictive value = (TP/(TP + FP)) | 79.4% |
| Negative predictive value = (TN/(TN + FN)) | 100.0% |

The table above shows analyses based on the 92 clinical samples that were tested. The antigen cut-off concentration is 7.25 ng/mL where samples below this concentration are considered negative and above this concentration are considered positive. Tests with visual grades that were ≥2 were assigned a positive result and visual grades that are ≤1 were assigned a negative result. This assay shows high sensitivity and high negative predictive value.

Results and analysis of the tested 92 plasma samples showed acceptable performance based on the client's desired performance specifications with the currently developed LFA for determination of sFlt-1 levels in plasma samples.

VII.vi. Results— 295 Plasma Sample Testing

295 individual samples were characterized and sent to DCN by the client to be tested in house. Testing in house included two LFA per sample each visually graded by two users and then placed in an Axxin AX-2X-S Lateral Flow Instrument for a reading. FIG. 20 shows visual grade data from testing the 295 characterized samples in house.

TABLE 9
The sensitivity and specificity, based on visual grading.

| | |
|---|---|
| Total negative samples tested (less than 7.5 ng/ml) | 201 |
| Total positive samples tested (greater than 7.5 ng/ml) | 94 |
| True negatives | 161 |
| True positives | 94 |
| False negatives | 0 |
| False positives | 40 |
| Sensitivity (TP/(TP + FN)) | 100.0% |
| Specificity (TN/(FP + TN)) | 80.1% |
| Positive predictive value = (TP/(TP + FP)) | 70.1% |
| Negative predictive value = (TN/(TN + FN)) | 100.0% |

The table above shows analyses based on the 295 clinical samples that were tested. The antigen cut-off concentration is 7.5 ng/mL where samples below this concentration are considered negative and above this concentration are considered positive. Samples with an average visual grade of ≥2 were assigned a positive result and average visual grades that are ≤1 were assigned a negative result. This assay shows high sensitivity and high negative predictive value.

TABLE 10
The sensitivity and specificity, based on axxin value.

| | |
|---|---|
| Total negative samples tested (less than 7.5 ng/ml) | 201 |
| Total positive samples tested (greater than 7.5 ng/ml) | 94 |
| True negatives | 170 |
| True positives | 94 |
| False negatives | 0 |
| False positives | 31 |
| Sensitivity (TP/(TP + FN)) | 100.0% |
| Specificity (TN/(FP + TN)) | 84.6% |
| Positive predictive value = (TP/(TP + FP)) | 75.2% |
| Negative predictive value = (TN/(TN + FN)) | 100.0% |

The table above shows analyses based on the 295 clinical samples that were tested. The antigen cut-off concentration is 7.5 ng/mL where samples below this concentration are considered negative and above this concentration are considered positive. Samples with an average Axxin test line peak value of ≥540 were assigned a positive result and any average value below this test line peak value were assigned a negative result. This assay shows high sensitivity and high negative predictive value.

TABLE 11
One-by-one comparison of visual grade assignments and average
Axxin peak value. (“WD”: sample missing due to withdrawn
subjects. “M”: sample missing due to not shipped.)

| | Visual Grade Assignments from 2 Users | | | | | | Average |
|---|---|---|---|---|---|---|---|
| | User 1 | | | User 2 | | | Axxin TL |
| Patient | Sample | | | Sample | | | Peak Value, |
| ID | ID | rep1 | rep2 | ID | rep1 | rep2 | n = 2 |
| P0001 | Serum 1 | 4 | 4 | Serum 1 | 5 | 5 | 1490 |
| P0002 | Serum 1 | 1 | 1 | Serum 1 | 2 | 2 | 405 |
| P0003 | Serum 1 | 4 | 4 | Serum 1 | 5 | 5 | 1165 |
| P0004 | Serum 1 | 1 | 0 | Serum 1 | 2 | 2 | 340 |
| P0005 | Serum 1 | 0 | 0 | Serum 1 | 0 | 0 | 190 |
| P0006 | Serum 1 | 1 | 2 | Serum 1 | 3 | 3 | 470 |
| P0007 | Serum 1 | 0 | 2 | Serum 1 | 0 | 2 | 380 |
| P0008 | Serum 1 | 2 | 3 | Serum 1 | 2 | 2 | 375 |
| P0009 | Serum 1 | 2 | 0 | Serum 1 | 1 | 0 | 285 |
| P0010 | Serum 1 | 0 | 0 | Serum 1 | 0 | 0 | 250 |
| P0011 | Serum 1 | 1 | 1 | Serum 1 | 1 | 0 | 380 |
| P0012 | Serum 1 | 6 | 6 | Serum 1 | 7 | 7 | 5030 |
| P0013 | Serum 1 | 4 | 3 | Serum 1 | 4 | 4 | 1090 |
| P0014 | Serum 1 | 0 | 0 | Serum 1 | 0 | 0 | 270 |
| P0015 | WD | — | — | WD | — | — | — |
| P0016 | Serum 1 | 0 | 0 | Serum 1 | 0 | 0 | 225 |
| P0017 | Serum 1 | 0 | 0 | Serum 1 | 1 | 1 | 260 |
| P0018 | Serum 1 | 1 | 2 | Serum 1 | 0 | 2 | 370 |
| P0019 | Serum 1 | 1 | 1 | Serum 1 | 2 | 1 | 345 |
| P0020 | Serum 1 | 5 | 5 | Serum 1 | 5 | 6 | 1875 |
| P0021 | Serum 1 | 0 | 0 | Serum 1 | 0 | 0 | 215 |
| P0022 | Serum 1 | 4 | 4 | Serum 1 | 4 | 5 | 1315 |
| P0023 | Serum 1 | 4 | 4 | Serum 1 | 4 | 4 | 1335 |
| P0024 | Serum 1 | 4 | 3 | Serum 1 | 3 | 3 | 1410 |
| P0025 | Serum 1 | 0 | 0 | Serum 1 | 1 | 0 | 300 |
| P0026 | WD | — | — | WD | — | — | — |
| P0027 | Serum 1 | 4 | 5 | Serum 1 | 4 | 4 | 1770 |
| P0028 | Serum 1 | 2 | 2 | Serum 1 | 2 | 2 | 430 |
| P0029 | Serum 1 | 0 | 1 | Serum 1 | 1 | 1 | 295 |
| P0030 | Serum 1 | 3 | 3 | Serum 1 | 3 | 4 | 800 |
| P0031 | Serum 1 | 2 | 0 | Serum 1 | 2 | 1 | 380 |
| P0032 | Serum 1 | 4 | 5 | Serum 1 | 6 | 5 | 2115 |
| P0033 | Serum 1 | 2 | 2 | Serum 1 | 3 | 2 | 425 |
| P0034 | Serum 1 | 1 | 1 | Serum 1 | 2 | 1 | * |
| P0035 | Serum 1 | 0 | 0 | Serum 1 | 0 | 0 | 245 |
| P0036 | Serum 1 | 6 | 6 | Serum 1 | 7 | 7 | 3965 |
| P0037 | Serum 1 | 0 | 0 | Serum 1 | 0 | 0 | 220 |
| P0038 | Serum 1 | 3 | 3 | Serum 1 | 2 | 3 | 905 |
| P0039 | Serum 1 | 5 | 6 | Serum 1 | 6 | 6 | 3375 |
| P0040 | Serum 1 | 1 | 0 | Serum 1 | 0 | 1 | 270 |
| P0041 | Serum 1 | 3 | 4 | Serum 1 | 4 | 4 | 1565 |
| P0042 | Serum 1 | 0 | 0 | Serum 1 | 0 | 0 | 270 |
| P0043 | Serum 1 | 0 | 0 | Serum 1 | 1 | 0 | 315 |
| P0044 | Serum 1 | 0 | 0 | Serum 1 | 1 | 0 | 325 |
| P0045 | Serum 1 | 0 | 2 | Serum 1 | 0 | 1 | 390 |
| P0046 | Serum 1 | 1 | 1 | Serum 1 | 0 | 1 | 485 |
| P0047 | Serum 1 | 0 | 1 | Serum 1 | 0 | 0 | 210 |
| 02-001 | Serum 1 | 0 | 0 | Serum 1 | 0 | 0 | 210 |
| 02-002 | Serum 1 | 4 | 4 | Serum 1 | 4 | 4 | 1605 |
| 02-003 | Serum 1 | 3 | 3 | Serum 1 | 3 | 3 | 890 |
| 02-004 | Serum 1 | 2 | 2 | Serum 1 | 2 | 2 | 570 |
| 02-005 | Serum 1 | 3 | 3 | Serum 1 | 3 | 3 | 790 |
| 02-006 | Serum 1 | 5 | 6 | Serum 1 | 6 | 6 | 3465 |
| 02-007 | Serum 1 | 3 | 2 | Serum 1 | 3 | 3 | 750 |
| 02-008 | Serum 1 | 4 | 4 | Serum 1 | 4 | 4 | 1310 |
| 02-009 | Serum 1 | 1 | 2 | Serum 1 | 1 | 1 | 355 |
| 02-010 | Serum 1 | 4 | 4 | Serum 1 | 4 | 4 | 1890 |
| 02-011 | Serum 1 | 4 | 5 | Serum 1 | 5 | 6 | 2980 |
| 02-012 | Serum 1 | 5 | 4 | Serum 1 | 5 | 4 | 2100 |
| 02-013 | Serum 1 | 5 | 4 | Serum 1 | 4 | 4 | 2075 |
| 02-014 | Serum 1 | 3 | 3 | Serum 1 | 3 | 3 | 810 |
| 03-001 | Serum 1 | 4 | 3 | Serum 1 | 4 | 4 | 1360 |
| 03-002 | Serum 1 | 3 | 3 | Serum 1 | 2 | 3 | 710 |
| 03-003 | Serum 1 | 0 | 0 | Serum 1 | 0 | 0 | 225 |
| 03-004 | Serum 1 | 3 | 3 | Serum 1 | 3 | 3 | 1015 |
| 03-005 | Serum 1 | 0 | 0 | Serum 1 | 1 | 0 | 305 |
| 03-006 | WD | — | — | WD | — | — | — |
| 03-007 | WD | — | — | WD | — | — | — |
| 03-008 | Serum 1 | 4 | 4 | Serum 1 | 4 | 4 | 1680 |
| 03-009 | Serum 1 | 3 | 3 | Serum 1 | 3 | 3 | 1085 |
| 03-010 | Serum 1 | 1 | 2 | Serum 1 | 2 | 2 | 470 |
| 03-011 | Serum 1 | 1 | 2 | Serum 1 | 2 | 2 | 455 |
| 03-012 | Serum 1 | 0 | 0 | Serum 1 | 2 | 1 | 255 |
| 03-013 | Serum 1 | 0 | 0 | Serum 1 | 0 | 0 | 255 |
| 03-014 | Serum 1 | 2 | 2 | Serum 1 | 3 | 2 | 655 |
| 03-015 | Serum 1 | 1 | 1 | Serum 1 | 0 | 1 | 365 |
| 03-016 | Serum 1 | 3 | 4 | Serum 1 | 3 | 4 | 1060 |
| 03-017 | Serum 1 | 4 | 5 | Serum 1 | 4 | 5 | 1645 |
| 03-018 | Serum 1 | 3 | 4 | Serum 1 | 3 | 3 | 1035 |
| 03-019 | Serum 1 | 0 | 1 | Serum 1 | 0 | 1 | 240 |
| 03-020 | Serum 1 | 6 | 7 | Serum 1 | 7 | 7 | 4905 |
| 03-021 | Serum 1 | 4 | 4 | Serum 1 | 4 | 4 | 1625 |
| 03-022 | Serum 1 | 0 | 0 | Serum 1 | 0 | 0 | 260 |
| 03-023 | Serum 1 | 2 | 1 | Serum 1 | 2 | 1 | 560 |
| 03-024 | Serum 1 | 0 | 0 | Serum 1 | 0 | 0 | 265 |
| 03-025 | Serum 1 | 2 | 0 | Serum 1 | 1 | 1 | 255 |
| 03-026 | Serum 1 | 2 | 0 | Serum 1 | 2 | 0 | 280 |
| 03-027 | Serum 1 | 0 | 0 | Serum 1 | 1 | 0 | 265 |
| 03-028 | Serum 1 | 0 | 0 | Serum 1 | 0 | 0 | 215 |
| 03-029 | Serum 1 | 3 | 2 | Serum 1 | 3 | 3 | 870 |
| 03-030 | Serum 1 | 0 | 1 | Serum 1 | 1 | 0 | 260 |
| 03-031 | Serum 1 | 2 | 1 | Serum 1 | 1 | 1 | 370 |
| 03-032 | Serum 1 | 1 | 0 | Serum 1 | 2 | 1 | 330 |
| 03-033 | Serum 1 | 5 | 4 | Serum 1 | 5 | 4 | 2020 |
| 03-034 | Serum 1 | 4 | 3 | Serum 1 | 5 | 4 | 1850 |
| 03-035 | Serum 1 | 0 | 1 | Serum 1 | 0 | 2 | 360 |
| 03-036 | Serum 1 | 0 | 0 | Serum 1 | 0 | 0 | 275 |
| 03-037 | Serum 1 | 1 | 2 | Serum 1 | 0 | 1 | * |
| 03-038 | Serum 1 | 1 | 3 | Serum 1 | 2 | 3 | 970 |
| 03-039 | Serum 1 | 0 | 0 | Serum 1 | 0 | 0 | 245 |
| 03-040 | Serum 1 | 3 | 2 | Serum 1 | 2 | 2 | 470 |
| 03-041 | Serum 1 | 1 | 0 | Serum 1 | 1 | 0 | 255 |
| 03-042 | Serum 1 | 3 | 2 | Serum 1 | 3 | 2 | 805 |
| 03-043 | WD | — | — | WD | — | — | — |
| 03-044 | Serum 1 | 0 | 1 | Serum 1 | 1 | 0 | 325 |
| 03-045 | Serum 1 | 0 | 1 | Serum 1 | 1 | 1 | 320 |
| 03-046 | Serum 1 | 3 | 3 | Serum 1 | 3 | 4 | 1050 |
| 03-047 | Serum 1 | 0 | 1 | Serum 1 | 0 | 0 | 275 |
| 03-048 | Serum 1 | 2 | 3 | Serum 1 | 2 | 3 | 545 |
| 03-049 | Serum 1 | 0 | 0 | Serum 1 | 0 | 0 | 240 |
| 03-050 | Serum 1 | 0 | 0 | Serum 1 | 0 | 0 | 210 |
| 03-051 | Serum 1 | 2 | 3 | Serum 1 | 2 | 2 | 500 |
| 03-052 | Serum 1 | 1 | 1 | Serum 1 | 1 | 0 | 350 |
| 03-053 | Serum 1 | 4 | 4 | Serum 1 | 3 | 4 | 1380 |
| 03-054 | Serum 1 | 3 | 3 | Serum 1 | 2 | 3 | 490 |
| 03-055 | Serum 1 | 0 | 0 | Serum 1 | 1 | 0 | 210 |
| 03-056 | Serum 1 | 1 | 1 | Serum 1 | 1 | 0 | 275 |
| 03-057 | Serum 1 | 4 | 3 | Serum 1 | 4 | 3 | 1075 |
| 03-058 | Serum 1 | 1 | 2 | Serum 1 | 1 | 2 | 415 |
| 04-001 | Serum 1 | 1 | 0 | Serum 1 | 1 | 0 | 380 |
| 04-002 | Serum 1 | 4 | 4 | Serum 1 | 4 | 4 | 1985 |
| 04-003 | Serum 1 | 4 | 5 | Serum 1 | 4 | 5 | 2205 |
| 04-004 | Serum 1 | 2 | 2 | Serum 1 | 1 | 2 | 520 |
| 04-005 | Serum 1 | 6 | 5 | Serum 1 | 6 | 5 | 3435 |
| 04-006 | Serum 1 | 0 | 0 | Serum 1 | 0 | 1 | 290 |
| 04-007 | Serum 1 | 3 | 4 | Serum 1 | 3 | 4 | 1280 |
| 04-008 | Serum 1 | 2 | 1 | Serum 1 | 2 | 2 | 455 |
| 04-009 | Serum 1 | 0 | 0 | Serum 1 | 0 | 0 | 245 |
| 04-010 | Serum 1 | 1 | 1 | Serum 1 | 1 | 0 | 330 |
| 04-011 | Serum 1 | 5 | 5 | Serum 1 | 5 | 6 | 2905 |
| 04-012 | Serum 1 | 1 | 1 | Serum 1 | 2 | 2 | 470 |
| 04-013 | Serum 1 | 3 | 2 | Serum 1 | 3 | 3 | 895 |
| 04-014 | Serum 1 | 3 | 1 | Serum 1 | 2 | 2 | 495 |
| 04-015 | Serum 1 | 4 | 5 | Serum 1 | 4 | 4 | 2035 |
| 04-016 | Serum 1 | 0 | 0 | Serum 1 | 0 | 0 | 220 |
| 04-017 | Serum 1 | 2 | 2 | Serum 1 | 2 | 2 | 550 |
| 05-001 | WD | — | — | WD | — | — | — |
| 05-002 | Serum 1 | 0 | 0 | Serum 1 | 0 | 0 | 195 |
| 05-003 | Serum 1 | 0 | 0 | Serum 1 | 0 | 0 | 170 |
| 05-004 | WD | — | — | WD | — | — | — |
| 05-005 | Serum 1 | 0 | 0 | Serum 1 | 0 | 0 | 260 |
| 05-006 | Serum 1 | 0 | 0 | Serum 1 | 0 | 0 | 295 |
| 05-007 | Serum 1 | 0 | 0 | Serum 1 | 0 | 0 | 230 |
| 05-008 | Serum 1 | 0 | 0 | Serum 1 | 0 | 0 | 280 |
| 05-009 | Serum 1 | 5 | 5 | Serum 1 | 5 | 4 | 2385 |
| 05-010 | Serum 1 | 0 | 0 | Serum 1 | 1 | 1 | 300 |
| 05-011 | Serum 1 | 0 | 1 | Serum 1 | 0 | 0 | 270 |
| 05-012 | WD | — | — | WD | — | — | — |
| 05-013 | Serum 1 | 3 | 2 | Serum 1 | 3 | 2 | 865 |
| 05-014 | Serum 1 | 2 | 0 | Serum 1 | 2 | 1 | 470 |
| 05-015 | Serum 1 | 2 | 2 | Serum 1 | 2 | 2 | 590 |
| 05-016 | Serum 1 | 3 | 3 | Serum 1 | 3 | 4 | 1460 |
| 05-017 | Serum 1 | 0 | 0 | Serum 1 | 0 | 0 | 255 |
| 05-018 | Serum 1 | 0 | 0 | Serum 1 | 0 | 0 | 225 |
| 05-019 | Serum 1 | 3 | 3 | Serum 1 | 3 | 2 | 790 |
| 05-020 | Serum 1 | 0 | 0 | Serum 1 | 1 | 0 | 240 |
| 05-021 | Serum 1 | 2 | 3 | Serum 1 | 3 | 3 | 720 |
| 05-022 | Serum 1 | 1 | 0 | Serum 1 | 2 | 2 | 420 |
| 05-023 | Serum 1 | 2 | 2 | Serum 1 | 3 | 3 | 735 |
| 06-001 | Serum 1 | 3 | 3 | Serum 1 | 3 | 3 | 605 |
| 06-002 | Serum 1 | 1 | 0 | Serum 1 | 1 | 0 | 405 |
| 06-003 | Serum 1 | 4 | 4 | Serum 1 | 4 | 4 | 1435 |
| 06-004 | Serum 1 | 4 | 5 | Serum 1 | 5 | 5 | 2170 |
| 06-005 | Serum 1 | 1 | 3 | Serum 1 | 2 | 3 | 690 |
| 06-006 | Serum 1 | 0 | 0 | Serum 1 | 0 | 0 | 215 |
| 06-007 | Serum 1 | 3 | 4 | Serum 1 | 3 | 4 | 1035 |
| 06-008 | Serum 1 | 2 | 2 | Serum 1 | 2 | 3 | 610 |
| 06-009 | Serum 1 | 2 | 1 | Serum 1 | 3 | 2 | 500 |
| 06-010 | WD | — | — | WD | — | — | — |
| 06-011 | Serum 1 | 1 | 0 | Serum 1 | 1 | 0 | 250 |
| 06-012 | Serum 1 | 2 | 3 | Serum 1 | 3 | 3 | 730 |
| 07-001 | Serum 1 | 5 | 4 | Serum 1 | 5 | 4 | 2080 |
| 07-002 | Serum 1 | 3 | 3 | Serum 1 | 4 | 3 | 960 |
| 07-003 | Serum 1 | 5 | 4 | Serum 1 | 5 | 5 | * |
| 07-004 | Serum 1 | 0 | 0 | Serum 1 | 0 | 0 | 200 |
| 07-005 | Serum 1 | 2 | 3 | Serum 1 | 3 | 3 | 680 |
| 07-006 | Serum 1 | 2 | 3 | Serum 1 | 3 | 3 | 780 |
| 07-007 | Serum 1 | 6 | 6 | Serum 1 | 5 | 6 | 3300 |
| 07-008 | WD | — | — | WD | — | — | — |
| 07-009 | WD | — | — | WD | — | — | — |
| 07-010 | Serum 1 | 4 | 3 | Serum 1 | 4 | 4 | 890 |
| 07-011 | Serum 1 | 2 | 2 | Serum 1 | 2 | 2 | 605 |
| 07-012 | M | — | — | M | — | — | — |
| 08-001 | WD | — | — | WD | — | — | — |
| 08-002 | Serum 1 | 3 | 4 | Serum 1 | 4 | 4 | 1110 |
| 08-003 | Serum 1 | NA | NA | Serum 1 | NA | NA | — |
| 08-004 | Serum 1 | 0 | 1 | Serum 1 | 0 | 0 | 240 |
| 08-005 | Serum 1 | 0 | 1 | Serum 1 | 0 | 0 | 160 |
| 08-006 | Serum 1 | 0 | 1 | Serum 1 | 1 | 1 | 425 |
| 08-007 | Serum 1 | 0 | 0 | Serum 1 | 1 | 0 | 210 |
| 08-008 | Serum 1 | 2 | 1 | Serum 1 | 2 | 1 | 470 |
| 08-009 | Serum 1 | 3 | 4 | Serum 1 | 4 | 4 | 1445 |
| 08-010 | Serum 1 | 2 | 2 | Serum 1 | 3 | 2 | 615 |
| 08-011 | Serum 1 | 3 | 3 | Serum 1 | 4 | 3 | 1090 |
| 09-001 | Serum 1 | 0 | 1 | Serum 1 | 0 | 1 | 210 |
| 09-002 | Serum 1 | 1 | 1 | Serum 1 | 1 | 1 | 435 |
| 09-003 | Serum 1 | 0 | 0 | Serum 1 | 0 | 0 | 210 |
| 09-004 | Serum 1 | 1 | 0 | Serum 1 | 0 | 0 | 325 |
| 09-005 | Serum 1 | 3 | 3 | Serum 1 | 3 | 3 | 875 |
| 10-001 | Serum 1 | 0 | 0 | Serum 1 | 0 | 0 | 315 |
| 10-002 | Serum 1 | 2 | 1 | Serum 1 | 2 | 2 | 640 |
| 10-003 | WD | — | — | WD | — | — | — |
| 10-004 | Serum 1 | 0 | 1 | Serum 1 | 1 | 1 | 450 |
| 10-005 | Serum 1 | 0 | 0 | Serum 1 | 0 | 0 | 225 |
| 10-006 | Serum 1 | 0 | 2 | Serum 1 | 1 | 2 | 320 |
| 10-007 | Serum 1 | 1 | 2 | Serum 1 | 2 | 2 | 585 |
| 10-008 | Serum 1 | 1 | 2 | Serum 1 | 1 | 1 | 425 |
| 10-009 | WD | — | — | WD | — | — | — |
| 10-010 | Serum 1 | 2 | 1 | Serum 1 | 2 | 2 | 460 |
| 10-011 | Serum 1 | 1 | 1 | Serum 1 | 0 | 2 | 435 |
| 10-012 | Serum 1 | 1 | 0 | Serum 1 | 0 | 1 | 375 |
| 10-013 | Serum 1 | 1 | 1 | Serum 1 | 1 | 1 | 375 |
| 10-014 | Serum 1 | 2 | 2 | Serum 1 | 2 | 2 | 560 |
| 10-015 | Serum 1 | 4 | 3 | Serum 1 | 4 | 4 | 1220 |
| 10-016 | Serum 1 | 4 | 4 | Serum 1 | 4 | 4 | 1190 |
| 10-017 | Serum 1 | 4 | 4 | Serum 1 | 5 | 4 | 1695 |
| 10-018 | Serum 1 | 0 | 0 | Serum 1 | 0 | 0 | 130 |
| 10-019 | Serum 1 | 4 | 3 | Serum 1 | 4 | 3 | 1495 |
| 10-020 | Serum 1 | 1 | 0 | Serum 1 | 0 | 0 | 210 |
| 11-001 | Serum 1 | 4 | 3 | Serum 1 | 4 | 4 | 1465 |
| 11-002 | WD | — | — | WD | — | — | — |
| 11-003 | Serum 1 | 1 | 0 | Serum 1 | 1 | 0 | 260 |
| 11-004 | Serum 1 | 0 | 0 | Serum 1 | 0 | 0 | 230 |
| 11-005 | Serum 1 | 1 | 0 | Serum 1 | 1 | 1 | 260 |
| 11-006 | WD | — | — | WD | — | — | — |
| 11-007 | Serum 1 | 0 | 0 | Serum 1 | 0 | 0 | 255 |
| 11-008 | Serum 1 | 1 | 1 | Serum 1 | 1 | 1 | 260 |
| 11-009 | Serum 1 | 4 | 4 | Serum 1 | 5 | 4 | 1530 |
| 11-010 | Serum 1 | 0 | 1 | Serum 1 | 1 | 0 | 320 |
| 11-011 | Serum 1 | 3 | 3 | Serum 1 | 4 | 3 | 1060 |
| 11-012 | Serum 1 | 3 | 3 | Serum 1 | 3 | 3 | 825 |
| 11-013 | Serum 1 | 0 | 0 | Serum 1 | 1 | 0 | 195 |
| 11-014 | Serum 1 | 0 | 0 | Serum 1 | 0 | 0 | 260 |
| 11-015 | Serum 1 | 3 | 4 | Serum 1 | 4 | 5 | 1470 |
| 11-016 | Serum 1 | 4 | 4 | Serum 1 | 4 | 4 | 1540 |
| 11-017 | Serum 1 | 1 | 0 | Serum 1 | 1 | 1 | 280 |
| 11-018 | Serum 1 | 2 | 2 | Serum 1 | 2 | 2 | 450 |
| 11-019 | Serum 1 | 3 | 3 | Serum 1 | 4 | 4 | 1360 |
| 11-020 | Serum 1 | 2 | 2 | Serum 1 | 3 | 3 | 720 |
| 11-021 | Serum 1 | 0 | 0 | Serum 1 | 0 | 1 | 255 |
| 11-022 | Serum 1 | 0 | 0 | Serum 1 | 0 | 0 | 200 |
| 11-023 | Serum 1 | 5 | 4 | Serum 1 | 5 | 4 | 2115 |
| 11-024 | Serum 1 | 1 | 0 | Serum 1 | 1 | 1 | 270 |
| 11-025 | Serum 1 | 3 | 4 | Serum 1 | 3 | 4 | 985 |
| 11-026 | Serum 1 | 0 | 0 | Serum 1 | 0 | 0 | 200 |
| 11-027 | Serum 1 | 0 | 0 | Serum 1 | 0 | 0 | 215 |
| 11-028 | Serum 1 | 0 | 0 | Serum 1 | 0 | 0 | 255 |
| 11-029 | Serum 1 | 1 | 0 | Serum 1 | 0 | 1 | 255 |
| 11-030 | Serum 1 | 1 | 1 | Serum 1 | 0 | 1 | 295 |
| 11-031 | Serum 1 | 3 | 2 | Serum 1 | 4 | 3 | 820 |
| 11-032 | Serum 1 | 1 | 2 | Serum 1 | 1 | 2 | 525 |
| 11-033 | Serum 1 | 5 | 5 | Serum 1 | 5 | 6 | 2880 |
| 11-034 | Serum 1 | 0 | 0 | Serum 1 | 0 | 0 | 260 |
| 11-035 | Serum 1 | 3 | 3 | Serum 1 | 3 | 3 | 725 |
| 11-036 | Serum 1 | 1 | 0 | Serum 1 | 1 | 0 | 275 |
| 11-037 | Serum 1 | 0 | 1 | Serum 1 | 0 | 0 | 275 |
| 11-038 | Serum 1 | 1 | 1 | Serum 1 | 0 | 1 | 290 |
| 11-039 | Serum 1 | 4 | 3 | Serum 1 | 4 | 3 | 1445 |
| 11-040 | WD | — | — | WD | — | — | — |
| 11-041 | Serum 1 | 0 | 0 | Serum 1 | 0 | 0 | 240 |
| 11-042 | Serum 1 | 1 | 0 | Serum 1 | 1 | 1 | 290 |
| 11-043 | Serum 1 | 0 | 0 | Serum 1 | 0 | 1 | 305 |
| 11-044 | Serum 1 | 1 | 1 | Serum 1 | 2 | 2 | 455 |
| 11-045 | Serum 1 | 3 | 4 | Serum 1 | 4 | 4 | 1425 |
| 11-046 | Serum 1 | 1 | 2 | Serum 1 | 2 | 3 | 430 |
| 11-047 | Serum 1 | 5 | 3 | Serum 1 | 5 | 4 | 1780 |
| 11-048 | Serum 1 | 0 | 1 | Serum 1 | 1 | 2 | 355 |
| 11-049 | Serum 1 | 0 | 0 | Serum 1 | 0 | 0 | 240 |
| 11-050 | Serum 1 | 0 | 0 | Serum 1 | 0 | 1 | 325 |
| 11-051 | Serum 1 | 2 | 3 | Serum 1 | 3 | 3 | 775 |
| 11-052 | Serum 1 | 2 | 2 | Serum 1 | 3 | 3 | 710 |
| 11-053 | Serum 1 | 5 | 5 | Serum 1 | 5 | 5 | 2440 |
| 11-054 | Serum 1 | 0 | 0 | Serum 1 | 0 | 0 | 240 |
| 11-055 | Serum 1 | 3 | 3 | Serum 1 | 5 | 4 | 1475 |
| 11-056 | Serum 1 | 0 | 0 | Serum 1 | 1 | 0 | 320 |
| 11-057 | Serum 1 | 3 | 4 | Serum 1 | 4 | 4 | 1630 |
| 11-058 | Serum 1 | 1 | 1 | Serum 1 | 1 | 2 | 240 |
| 11-059 | Serum 1 | 0 | 1 | Serum 1 | 1 | 1 | 330 |
| 11-060 | Serum 1 | 2 | 2 | Serum 1 | 2 | 3 | 655 |
| 11-061 | Serum 1 | 4 | 5 | Serum 1 | 5 | 5 | 2080 |
| 11-062 | Serum 1 | 0 | 1 | Serum 1 | 1 | 1 | 400 |
| 11-063 | Serum 1 | 1 | 2 | Serum 1 | 2 | 3 | 540 |
| 11-064 | Serum 1 | 5 | 6 | Serum 1 | 5 | 5 | 2525 |
| 11-065 | Serum 1 | 1 | 1 | Serum 1 | 1 | 1 | 330 |
| 11-066 | Serum 1 | 2 | 1 | Serum 1 | 2 | 1 | 415 |
| 11-067 | Serum 1 | 3 | 3 | Serum 1 | 3 | 4 | 1090 |
| 11-068 | Serum 1 | 6 | 6 | Serum 1 | 6 | 6 | 3675 |
| 11-069 | Serum 1 | 4 | 4 | Serum 1 | 3 | 4 | 1525 |
| 11-070 | Serum 1 | 0 | 0 | Serum 1 | 0 | 0 | 195 |
| 11-071 | Serum 1 | 3 | 3 | Serum 1 | 3 | 3 | 1110 |
| 11-072 | Serum 1 | 3 | 2 | Serum 1 | 3 | 2 | 575 |
| 12-001 | Serum 1 | 2 | 2 | Serum 1 | 2 | 2 | 475 |
| 12-002 | Serum 1 | 3 | 4 | Serum 1 | 4 | 4 | 1170 |
| 12-003 | Serum 1 | 0 | 1 | Serum 1 | 0 | 1 | 260 |
| 12-004 | Serum 1 | 0 | 0 | Serum 1 | 0 | 0 | 210 |
| 12-005 | Serum 1 | 0 | 0 | Serum 1 | 0 | 0 | 180 |
| 12-006 | Serum 1 | 5 | 4 | Serum 1 | 5 | 5 | 2485 |
| 12-007 | Serum 1 | 4 | 3 | Serum 1 | 4 | 3 | 1110 |
| 12-008 | Serum 1 | 1 | 0 | Serum 1 | 0 | 0 | 260 |
| 12-009 | Serum 1 | 1 | 0 | Serum 1 | 2 | 1 | 340 |
| 12-010 | Serum 1 | 0 | 0 | Serum 1 | 0 | 0 | 210 |
| 12-011 | Serum 1 | 4 | 5 | Serum 1 | 4 | 5 | 2175 |
| 12-012 | Serum 1 | 1 | 1 | Serum 1 | 1 | 1 | 340 |
| 12-013 | Serum 1 | ** | ** | Serum 1 | 0 | 1 | 275 |
| 12-014 | Serum 1 | ** | ** | Serum 1 | 0 | 0 | 265 |
| 12-015 | Serum 1 | ** | ** | Serum 1 | 5 | 5 | 2420 |
| 12-016 | Serum 1 | ** | ** | Serum 1 | 0 | 0 | 265 |
| 12-017 | Serum 1 | ** | ** | Serum 1 | 0 | 0 | 220 |
| 12-018 | Serum 1 | ** | ** | Serum 1 | 1 | 1 | 345 |
| 12-019 | Serum 1 | ** | ** | Serum 1 | 2 | 1 | 430 |
| 12-020 | Serum 1 | ** | ** | Serum 1 | 0 | 0 | 285 |
| 12-021 | M | — | — | M | — | — | — |
| 12-022 | M | — | — | M | — | — | — |
| 14-001 | WD | — | — | WD | — | — | — |
| 14-002 | Serum 1 | NA | NA | Serum 1 | 2 | 1 | 425 |
| 14-003 | Serum 1 | NA | NA | Serum 1 | 0 | 0 | 230 |
| 14-004 | Serum 1 | NA | NA | Serum 1 | 2 | 2 | 510 |

*Reader data not obtained due to reader problems
**Note:
User 1 was not available to read samples 12-013 through 14-004

VII.vii. Results—Exploring the Saliva Matrix

Saliva Sample Screen with Polystreptavidin Test Line (FIG. 1I): Results were obtained from full strips run for 30 minutes with 15 uL of spiked and unspiked individual saliva samples. Samples were spiked to a final concentration of 25 ng/mL with recombinant antigen. In one case a strip was tested using 50 uL of sample instead of 15 uL. This was performed to examine the effect of sample volume on the performance of the assay but did not show an impact of the result. 1.67 uL of 45 ng/mL Biotinylated DuoSet Detection antibody, and 5 uL of 0.1% DuoSet Capture latex conjugate solution per strip. CN95 membrane. Polystreptavidin test line at 0.75 mg/mL concentration. All 5 saliva samples showed differentiation in signal from unspiked to spiked but with varying levels of signal. Variation of the unspiked sample is likely due to differences in composition in individual sample matrix leading to NSB.

Spiking Saliva with High sFlt-1 Level Plasma (FIG. 1J): Results were obtained from full strips run with 15 μL of spiked and unspiked saliva sample from two different donors. Saliva was spiked using a small volume of characterized plasma sample with a concentration of 23.96 ng/mL in order to reach an estimated 3 ng/mL final concentration in the saliva samples. This allows for the use of endogenous sFLT-1 versus recombinant antigen, since they have previously been shown to perform differently in the assay. Differentiation can be observed between the spiked and unspiked samples for both saliva donors.

Sample Panel 6 (FIG. 1K): Results were obtained from strips run laterally with 100 uL of characterized unaltered saliva sample from multiple donors. 10.5 uL/cm 0.1% DuoSet Capture Latex with and without 150 ug/mL Goat IgG in diluent. 6 uL/cm 22.5 ug/mL DuoSet Detection Solution with and without 150 ug/mL Goat IgG in diluent. CN95 membrane. 0.9 mg/mL Polystreptavidin Test Line. The testing examined assigned value correlation with obtained visual test line intensity while also examining Goat IgG as a potential additive to reduce sample variability and NSB. Overall, correlation of test line intensity and assigned value was limited and the addition of Goat IgG did not show an obvious improvement in the assay. Sample P0041 was not able to flow up the strip.

Initial results from preliminary saliva sample testing showed strong potential with the LFA developed for the plasma matrix. Preliminary studies show that the assay could pick up sFlt-1 in saliva when spiked with either recombinant antigen or characterized plasma; and identification of endogenous/native sFlt-1 in saliva based on characterization using the R&D Systems ELISA in the assay needed improvement.

### VIII. Further

While the assay currently uses a plasma sample, it can be adjusted to a format that can accept patient whole blood. This would involve the addition of a pad material on the test strip used to separate the red blood cells and placement of the test strip within a cassette.

With the assay/device, a saliva based LFA pre-eclampsia test will be validated using sFlt-1 antigen as the marker. A number of components were identified as important to the LFA product, which includes: MgCl₂, Goat IgG, and TCEP. Further, detergents such as PLURONIC and salt concentrations such as Magnesium sulfate described herein were critical to enable this device.

Details of each obtained results can be seen in examples below.

### Example 1. Large Sample Panel Screening with 92 Plasma Specimens to Examine and Validate Performance of Strips

A total of 92 samples were tested, and the concentrations of sFlt-1 in these samples were separately determined with a clinical chemistry platform. This study with samples of known concentrations of sFlt-1 was designed to examine the sensitivity of the strips and the reliability to predict positive and/or negative results based on visual examination of the color grade of the indication/test line. Overall results were very positive with the vast majority of samples correlating assigned value to visual grade. The general trend of increasing visual grade to increasing visual grade was also quite apparent. A handful of samples do appear to sit outside expected results, but this is a small minority out of the full panel tested in this experiment.

1. Assembled Strips on 80 mm Backing Card with Sample Pad:
Placed membrane on 80 mm backing card positioned 20 mm from bottom edge. The CN95 membrane was striped with 0.1 mg/mL Polystreptavidin+2% sucrose diluted into 1.6 mM Tris-HCl pH 8.0 as a “test line” and 0.3 mg/mL Goat anti-Mouse IgG antibody diluted in 40 mM Tris-HCl pH 8.0+50% sucrose as a “control line”, both applied at a concentration of 1.0 μL/cm.
Cut Ahlstrom Grade 243 pad to 22 mm and placed to overlap membrane by 2 mm—this pad is used as the wick, also referred to as the end flow region, typically downstream of indication region to wick fluid across the device.
Placed precut and sprayed Ahlstrom Grade 6614 to overlap membrane by 2 mm. Alhstrom Grade 6614 pad was sprayed with 0.1% 400 nm red latex 40:1 anti-hVEGFR DuoSet capture conjugate at 10.5 μL/cm, and sprayed with 22.5 μg/mL DuoSet detection antibody solution at 6 μL/cm—this pad is used at least as the development region, (also called “conjugate pad”), where the analyte binds with one or both antibodies (one modified with a detectable label, the other modified with a functional group that is capable of binding to the downstream streptavidin in the indication region) to form a complex.
Placed cut and blocked Ahlstrom Grade 1281 Sample pad to overlap conjugate pad by 2 mm. Ahlstrom Grade 1281 pad was blocked with 1M MgCl₂ and 0.5% PLURONIC F127—this pad is used in the sample receiving region.
Cut strips to 4 mm in width, perpendicular to the lateral flow direction.

### 2. General Test Procedure:

Pipetted 15 μL of designated sample into the glass tubes.
Dropped the prepared strips into the designated glass tube so that the sample pad was in direct contact with the sample.
Allowed for 30 seconds of strip development before injecting 100 μL of chase buffer (0.75% PLURONIC F127 in 1×PBS) into the bottom of each tube.

### Materials

Nitrocellulose membrane, CN95, has a large and open pore structure, which is a suitable substrate for chromatographic tests running on viscous or particle loaded samples. The fast lateral speed will allow getting a quick clearing of conjugate particles leading to a clean background.
Ahlstrom 243: 0.95 mm-180 mm/30 min: one of the thicker grades in the Ahlstrom thick paper series. Ahlstrom 243 offers both moderately high flow and absorption and is recommended for chromatography of heavy solute loading.
Ahlstrom 6614 is a synthetic conjugate pad that is hydrophilic when untreated and composed of small, homogeneous polyester fibers for increased uniformity. It is suitable for the application conjugate release pad. Because the media is naturally hydrophilic, pretreatment can be applied more easily and require a lower volume. Basis weight of Ahlstrom 6614 is 75 g/m², caliper 0.42 mm, wicking rate of 5 sec/2 cm, and water absorption capacity is 57 mg/cm².
Ahlstrom 1281 is composed of cotton/rayon fiber blend, with a basis weight of 70 g/m², caliper of 0.38 mm, wicking rate of 65 s/4 cm, and a waiter absorption of 60 mg/cm².
The signal reagent included an antibody-nanoparticle conjugate, where the antibody is anti-human VEGFR-1/Flt-1 polyclonal goat IgG antibody and the nanoparticle is colorimetric nanoparticle.
The immunosorbent solid phase capture reagent was coated with a titrated, limited amount of anti-human VEGFR-1 monoclonal mouse IgG antibody.
In embodiments of “thermometer”-type devices, sFlt-1 in a sample will be labeled with colored signal reagent (antibody-nanoparticle conjugate), and then be captured by the capture reagent, saturating the capture reagent as the antibody/antigen/antibody complex migrates across the capture reagent region. Since the capture antibody is limited in quantity and distributed across the solid phase along the direction of flow of wicked sample, the length of a zone that is saturated and colored by the antibody/antigen/antibody complex will be proportional to the concentration of the antigen and the length of this colored zone will thus reflect a semi-quantitative assessment of the concentration.
In various embodiments, an anti-human plasma protein (e.g., anti-IgG) is included for testing plasma or whole blood specimens as an internal control to indicate sample migration and color development.

### Results:

FIGS. 2-9 are images of full strips run each with 15 μL of plasma samples, on CN 95 membrane, with 0.1% DuoSet ELISA capture latex, 22.5 μg/mL DuoSet ELISA detection solution and polystreptavidin test line concentration of 0.1 mg/mL; and determined visual grades were annotated below each strip. FIGS. 10A and 10B are charts compiling the visual grades from images in FIGS. 2-9, plotted in ascending concentrations of sFlt-1.

Table 12 below shows analyses based on the 92 clinical samples that were tested. The antigen cut-off concentration is 7.25 ng/mL where samples below this concentration are considered negative and above this concentration are considered positive. Tests with visual grades that were ≥2 were assigned a positive result and visual grades that are ≤1 were assigned a negative result. This assay shows a high sensitivity and a high negative predictive value.

TABLE 12
Sensitivity and Specificity

| | |
|---|---|
| Total negative samples tested (less than 7.25 ng/ml) | 201 |
| Total positive samples tested (greater than 7.25 ng/ml) | 94 |
| True negatives (TN) | 161 |
| True positives (TP) | 94 |
| False negatives (FN) | 0 |
| False positives (FP) | 40 |
| Sensitivity (TP/(TP + FN)) | 100.0% |
| Specificity (TN/(FP + TN)) | 80.1% |
| Positive predictive value = (TP/(TP + FP)) | 70.1% |
| Negative predictive value = (TN/(TN + FN)) | 100.0% |

FIG. 18 shows analyses based on 295 clinical samples that were tested. The antigen cut-off concentration is 7.5 ng/mL where samples below this concentration are considered negative and above this concentration are considered positive. Samples with an average visual grade of ≥2 were assigned a positive results and average visual grade <2 assigned a negative result.

### Example 2. Different Buffers to Block Sample Pads (Sample Receiving Region of Devices)

Five different buffers were generated and used to block sample pad material (Ahlstrom Grade 1281) that was then dried. The five buffers tested were: (1) 0.6M MgCl₂ and 1% PLURONIC F127; (2) 1.0M MgCl₂ and 1% PLURONIC F127; (3) 1.5M MgCl₂ and 1% PLURONIC F127; (4) 1.0M MgCl₂ and 0.5% PLURONIC F127; (5) 1.0M MgCl₂ and 2% PLURONIC F127. Test sample for this study is plasma spiked with hVEGFR1 (sFlt-1), which was prepared by mixing 16.67 μL of 300 ng/mL hVEGFR-1 standard with 100 μL of pooled plasma sample. Test procedures included:
Pipetted 15 μL of designated sample into the glass tubes.
Spiked sample with 7.5 μL of 2.5 M MgCl₂ solution.
Mixed solution in tube.
Dropped the prepared strips into the designated glass tube so that the sample pad was in direct contact with the sample.
Allowed for 30 seconds of strip development before injecting 800_, of chase buffer (0.75% PLURONIC F127 in 1×PBS) into the bottom of each tube.
Allowed for an additional 4.5 minutes of strip development time (total of 5 minutes of development time).
Removed strips from the tubes and placed onto a backing.
Took an image of the strips.
Allowed for an additional 5 minutes of strip development time (total of 10 minutes of development time).
Removed strips from the tubes and placed onto a backing.
Took an image of the strips.
Allowed for an additional 5 minutes of strip development time (total of 15 minutes of development time).
Removed strips from the tubes and placed onto a backing.
Took an image of the strips.

This study was designed to test the performance of select buffers that included MgCl₂ and PLURONIC, which have been used as sample additives in related testing, when used to block the sample pad by soaking the pad and drying it in order to produce a completely dry lateral flow assay system. Initial testing used pooled plasma sample 3 (behaving ‘well’) and showed noticeable differences with regards to conjugate clear speed as well as test line intensity with the buffer using either the least amount of MgCl₂ or the most amount of PLURONIC producing the quickest clear and highest test lines.

Block buffers were than tested with pooled plasma sample 2 (behaving ‘poorly’). Results provide a different set of conclusions from pooled sample 1: indicating that higher salt provides good clearing and reduction in nonspecific binding.

Based on the results from both tested samples, buffer compositions of 1M MgCl₂ w/0.5% PLURONIC F127 and 1M MgCl₂ w/2% PLURONIC F127 were determined to perform the best based on conjugate release, conjugate aggregation, background clear, and test line intensity. Initial test results showed noticeable differences with regards to performance depending on the buffer used to block the sample pad. Further testing with these buffers using characterized plasma samples determined that the sample buffer composition of 1M MgCl₂ and 0.5% PLURONIC F127 provided the best ability to produce clean lines with a good conjugate clearing speed; therefore, it was selected for further development work.

Next, development moved to examine test line concentration and latex conjugate/biotinylated antibody solution spray rates in more depth. Results indicated that a lower latex/biotinylated antibody spray rate provided noticeable advantage with regards to conjugate clear speed without sacrificing a significant amount of sensitivity. Results also indicated that a polystreptavidin concentration between 0.1 and 0.05 mg/mL would likely be optimal.

Further testing with polystreptavidin concentration at 0.075 and 0.05 mg/mL in combination with the blocked sample pad showed good performance with regards to visual cutoff, but also suggested that the sample block reduced sensitivity significantly enough to warrant a further examination of the polystreptavidin concentration by increasing it above 0.075 mg/mL.

Testing with 0.1 mg/mL showed overall strong performance and quickly became the frontrunner. Due to these results, testing was performed with 0.25 mg/mL but sensitivity was too high. A test was therefore performed to determine a ‘sweet spot’ between 0.25 and 0.1 mg/mL by running strips with polystreptavidin at 0.175 mg/mL but results show little to no improvement.

### Example 3. Latex Conjugate Quality Control Study

The purpose of this study is to check the performance of newly prepared latex-antibody conjugates. A first lot to be studied was 1.06% 400 nm Red Latex 40:1 anti-hVEGFR DuoSet capture conjugate. A second lot to be studied was 0.86% 400 nm Red Latex 40:1 anti-hVEGFR DuoSet capture conjugate. A third lot to be studied was 1.13% 400 nm Red Latex 40:1 anti-hVEGFR DuoSet capture conjugate. Other reagents in this study included free (mobile) biotinylated DuoSet detection antibody, 900 μg/mL; and 1×PBS with 0.75% PLURONIC F127 as the chase buffer.

Three different lots of prepared conjugated were tested against each other using a single pooled plasma sample. The three lots all showed comparable and acceptable performance with regards to 0 ng/mL test line intensity, 50 ng/mL test line intensity, and background clearing.

### Example 4. Study of Spray Rates and Polystreptavidin Concentration

The purpose of this study was to conduct in depth examination of latex conjugate and biotinylated antibody solution spray rates in combination with test line concentration of polystreptavidin.

Reagents in this study included—
CN95 Striped 1.0 μL/cm with 0.25 mg/mL Streptavidin+1% Sucrose TL, 0.3 mg/mL GAM CL.
CN95 Striped 1.0 μL/cm with 0.10 mg/mL Streptavidin+1% Sucrose TL, 0.3 mg/mL GAM CL.
CN95 Striped 1.0 μL/cm with 0.05 mg/mL Streptavidin+1% Sucrose TL, 0.3 mg/mL GAM CL.
Ahlstrom Grade 6614 Sprayed with 0.1% 400 nm Red Latex 40:1 Anti-hVEGFR DuoSet Capture Conjugate at 10.5 μL/cm, Sprayed with 22.5 μg/mL DuoSet Detection Antibody Solution at 6 μL/cm
Ahlstrom Grade 6614 Sprayed with 0.1% 400 nm Red Latex 40:1 Anti-hVEGFR DuoSet Capture Conjugate at 12.5 μL/cm, Sprayed with 22.5 μg/mL DuoSet Detection Antibody Solution at 8 μL/cm
Ahlstrom Grade 6614 Sprayed with 0.1% 400 nm Red Latex 40:1 Anti-hVEGFR DuoSet Capture Conjugate at 14 μL/cm, Sprayed with 22.5 μg/mL DuoSet Detection Antibody Solution at 12 μL/cm
1×PBS with 0.75% PLURONIC F127
MgCl₂ in diH₂O

Procedures from this study included—
Pipetted 15 μL of designated sample into the glass tubes.
Spiked sample with 7.5 μL of 2.5M MgCl₂ solution.
Mixed solution in tube.
Dropped the prepared strips into the designated glass tube so that the sample pad was in direct contact with the sample.
Allowed for 30 seconds of strip development before injecting 80 μL of chase buffer (0.75% PLURONIC F127 in 1×PBS) into the bottom of each tube.
Allowed for an additional 14.5 minutes of strip development time (total of 15 minutes of development time).
Removed strips from the tubes and placed onto a backing.
Took an image of the strips.

The current experiment was designed to examine spray rates and test line concentration in order to fine tune assay performance. Based on results, test line concentration appears to have the most significant impact on system sensitivity. The 0.25 mg/mL test line concentration appears to have a visual cutoff around 1 ng/mL, 0.1 mg/mL test line concentration appears to have a visual cutoff around 3 ng/mL, and a 0.05 mg/mL test line concentration appears to have a visual cutoff around 6 ng/mL with the current LFA system.

A second significant conclusion that was made from the current experiment was that less latex conjugate and less biotinylated antibody appears to provide the best conjugate clearance speed without a significant sacrifice in sensitivity. This difference in clearance provides significant advantage with regards to required development time and ease of test and control line read.

### Example 5. Study of Test Line Concentration in Combination with Sample Pad Block

The purpose of this study was to continue testing selected sample pad block buffers in combination with select polystreptavidin concentration and currently selected conjugate pad spray rates.

Initial testing was performed to generate ‘control’ data for comparison of a blocked sample pad against an unblocked sample pad with freshly striped membrane and freshly sprayed conjugate pad. Initial results showed little to no noticeable difference between 0.05 mg/mL polystreptavidin and 0.075 mg/mL polystreptavidin that was striped on the CN95 membrane (indication region), which indicated that the concentration difference is not significant enough to alter performance.

Testing then moved to examination of two selected sample pad block buffers with both selected test line concentrations. As previously seen, sample pad block dampened test line formation when compared to unblocked sample pads especially with the highest concentration sample. Both blocks performed similarly although the 1M MgCl₂/0.5% PLURONIC F127 performed slightly more desirably based on clearance time and lack of test line formation for samples in the 4-5.4 ng/mL concentration even after allowing the strips to develop for 30 minutes.

Due to the lack of test line formation after 30 minutes it is likely that test line concentration can be increased to provide more sensitivity for samples above 6 ng/mL without increasing test line intensity in samples below 6 ng/mL.

### Example 6. Study to Fine Tune Test Line Concentration Based on Sample Pad Block

The purpose of this study was to determine if sensitivity can be improved by increasing test line concentration of streptavidin from 0.05 mg/mL in combination with selected sample pad block buffer.

The current experiment was designed to examine if a higher concentration of polystreptavidin could be used since the sample pad block had diminished some of the sensitivity of the LFA. Time course images from testing of the 0.1 mg/mL test line show an estimated visual cutoff around 8 ng/mL with a ten minute read. The line formation for the two samples that provided a completely different line morphology with previous testing at a lower polystreptavidin concentration also seems to have returned to a more expected morphology. Another observation with the 0.1 mg/mL test line was that even after a 15-minute development samples between 4-5.4 ng/mL did not generate visible lines.

Based on the results with 0.1 mg/mL it was decided that the concentration of 0.25 mg/mL should be tested to determine if more sensitivity could be obtained without lower the visual cutoff too dramatically. Results show visible lines at 10 minutes for the 5 ng/mL sample suggesting that the increase in polystreptavidin has decreased the visual cutoff of around 8 ng/mL with 0.1 mg/mL to around 5 ng/mL with 0.25 mg/mL.

### Example 7. Study of Various Streptavidin Concentrations in Test Line with Lower Solids and Biotinylated Antibody

The purpose of this study was to determine if sensitivity can be decreased using the high end of polystreptavidin concentration by decreasing the amount of latex and biotinylated antibody solution in the system.

One aspect of the current experiment was designed to examine if a higher concentration of polystreptavidin (0.25 mg/mL) could be used in combination with lower solids and biotinylated antibody and the sample pad block in an attempt to further fine tune the LFA performance by diminishing sensitivity slightly. Results show that while a decrease in latex conjugate and biotinylated antibody may reduce sensitivity slightly; decrease in the reagents does not reduce sensitivity to the desired visual cutoff of above 5 ng/mL.

Another aspect of the current experiment was designed to examine if a concentration of polystreptavidin between 0.1 and 0.25 mg/mL would provide optimal sensitivity. Results show that while a decrease in latex conjugate and biotinylated antibody appears to reduce sensitivity; the concentration of 0.175 mg/mL polystreptavidin concentration with decreased solids does not provide significant advantage over the previously tested 0.1 mg/mL concentration and still appears to produce a visual cutoff around 4-5 ng/mL.

### Example 8. Other Initial Studies

An initial study was conducted to examine if premixing a biotinylated anti-VEGFR-1 antibody (“biotinylated antibody”) with a latex bead-conjugated avidin (“conjugate”) has a significant impact on performance. Combining the conjugate and free biotinylated antibody before addition to an analyte-containing sample was compared to the conjugate addition to the sample first and to the biotinylated antibody addition to sample first. Results indicate that there may be a mild increase in test line intensity when the conjugate and the biontinylated antibody are combined before adding it to the sample; however, this appears to be accompanied by an increase in all binding including nonspecific, which hinted no real advantage over other addition sequences. Therefore, this study indicates that these reagents should be kept separate rather than premixed when dispensing on the pad for the dried strip format.

Another initial study was conducted to look into the washing times and testing of DuoSet detection antibody-streptavidin latex conjugate. DuoSet detection biotinylated antibody was combined with Streptavidin-Latex to use as the detector in the assay. After the antibody was added, it was washed 1 time to remove excess unbound biotin-Ab. In this experiment, the conjugate will be washed 3 more times to ensure that there was no excess Ab-biotin, and will be used in testing. A second tube of conjugate undergoing the same centrifugation steps will be carried out alongside the “test” conjugate and used as a control. Results showed that the introduction of additional wash steps to the 0.1% latex conjugate did not result in an increase in assay sensitivity. Free unbound biotinylated antibody is not causing reduced test line intensity.

Subsequent to studying a device prototype containing only a membrane and a wicking pad (“half” strip), another study was designed to test a “full” strip prototype which further included a conjugate pad. The conjugate and biotinylated antibody would eventually be dispensed and dried onto the conjugate pad. The first test was performed to simply examine the effect of the pad addition while keeping all the reagents as wet reagents. Results showed overall similar performance between the half and full strip with a hotspot appearing with the full strip which is likely due to the difference in flow and mixing created by the use of the pad; dose response however is similar. Next, testing moved to spotting conjugate and biotinylated antibody onto the 8951 pad in different locations: conjugate at the start of the strip or antibody at the start of the strip. Results were overall positive: nonspecific binding was kept at a minimum and intensity at 12.5 ng/mL provided acceptable levels of separation; however, test line formation was not uniform with a number of spots appearing. This is likely due to release and flow through the 8951 pad and may be resolvable with pad material adjustment. A third configuration of reagents was then tested: premixing chase buffer, conjugate, and biotinylated antibody and applying it to the strip which had sample spotted on the 8951 pad. Results were similar to the other two configurations with regards to dose response but line formation appeared to be more uniform. The conjugate first configuration was then carried into plasma testing. As previously shown, nonspecific binding was increased overall but separation was obtained between 0 and 12.5 ng/mL.

Yet another initial study was performed to screen materials for the conjugate pads. Three pads were selected to compare with the initially selected 8951 pad: 6614 (polyester blend with binders), 8980 (glass fiber blend), and Standard 17 (glass fiber, different production method). Initial results showed significant differences between pads for the conjugate first configuration with 8980 and 6614 pads standing above the rest. Initial results for the premix configuration showed less overall differences indicating that more options may be available for this configuration. Based on the results of the conjugate first configuration, 6614 and 8951 pads were tested with plasma using both configurations. Results mirror previously obtained data: all binding increases with plasma; however, if the binding can be controlled the separation between 0 ng/mL and 12.5 ng/mL is significant and noticeably better when compared to the 8951 pad initially selected. Line formation with the 6614 and 8951 pads are also far more uniform for the conjugate first configuration than previous testing showed.

Another initial study was designed to examine the effect of tested additives (in “half” strips) on “full” strips using selected conjugate pads. Results for the 8980 pad do show noticeable differences when both PLURONIC and SYNPERONIC is included as compared to the control condition (no additive). Results for the 6614 pad, on the other hand, show little to no difference when additives are included with only the PVP58K showing only slight improvement. With the majority of selected additives showing mild to no improvement, other additives such as carrier proteins, Casein and BSA, are hypothesized to be suitable for inclusion in the devices and reduce non-specific binding.

Next, testing progressed into the investigation of adding a sample pad (for “sample receiving region”) to the strip and exploration of pad material to determine whether the presence of a physical sample filter before the sample mixes with assay reagents would reduce aggregation and nonspecific binding. Results did not show strong differences between the tested pads; where GE Standard 17 showed a mild improvement in decreasing visible aggregate at the conjugate pad/membrane interface and Ahlstrom 1281 showed little to no improvement. When further tested with the Ahlstrom 1281 as a sample pad, the examination of salts and surfactants as sample additives indicates that three possible additions result in decreased conjugate aggregation, reduction of non-specific binding, and background improvement: TRIS buffer, MgCl₂, and choline chloride.

Testing continued by titrating the biotinylated antibody in an attempt to further improve separation between positive and negative sample. Results from the titration of the biotinylated antibody down from 125 ng indicated a decrease in overall binding leading to less line formation with a negative sample. Testing this condition with the ‘good’ plasma showed a significant drop in test line intensity of the spiked plasma if previously obtained results are taken into account; however, reexamination of the strips after an extended 30-minute development time did show a potential for improved signal and separation.

### Example 9. Preliminary Testing with Saliva

Procedures:

1. Generated 45 ng/mL Free Biotinylated DuoSet Detection Antibody solution:
Combined 2 reconstituted stock antibody with 38 μL of 1×PBS/1% BSA solution.
Vortexed.

### 2. Generated Spiked Plasma Samples:

Spiked 40 μL of saliva with 3.33 μL of 300 ng/mL stock of hVEGFR standard to make 25 ng/mL sample.
Vortexed.

### 3. General Test Procedure:

Pipetted 15 μL of designated sample into the glass tubes.
Pipetted biotinylated DuoSet Detection antibody onto the strip near the start of the conjugate pad, pipetted 0.1% conjugate solution onto the conjugate pad approximately 3 mm from the pad/membrane interface.
Spiked sample with 7.5 μL of 2.5M MgCl₂ solution.
Spiked sample with 1.5 μL of 10% PLURONIC F127 solution.
Mixed solution in tube.
Dropped the prepared strips into the designated glass tube so that the sample pad was in direct contact with the sample.
Allowed for 30 seconds of strip development before injecting 80 μL of chase buffer (0.5% PLURONIC F127 in 1×PBS) into the bottom of each tube.
Allowed for an additional 29.5 minutes of strip development time (total of 10 minutes of development time).
Removed strips from the tubes and placed onto a backing.
Took an image of the strips.

Results from initial test with saliva show the ability for the assay to accommodate the saliva matrix as differentiation between negative and positive samples was observed for both streptavidin and polystreptavidin test line strips. An important observation made during testing was that the saliva sample ran significantly slower than the plasma sample previously tested.

### Example 10. Further Studies and Results with Saliva

Testing began by directly testing the lateral flow assay (LFA) as shown in Example 1 with in-house saliva donors. A total of ten saliva samples were tested spiked with recombinant sFlt-1 and unspiked. Results were overall positive with the test performing with similar characteristics as the plasma samples. One out of the ten samples tested showed significantly reduced flow but allowing for additional development time resulted in a result that matched the other nine samples.

Testing then moved to briefly examining the performance of the LFA by using different testing methods and volumes. Results were again positive and showed the potential for variable amounts of saliva to be used without significant negative impact on assay performance. It also showed the ability for use of a full saliva sample and no chase buffer, possibly with a modification to the strip construction.

Lastly, a testing method involving premixing the saliva sample in a buffer prior to applying to the test was evaluated. This method could help to reduce sensitivity of the assay since the assay currently appears to be too sensitive, as well as improve sample flow because of sample dilution. Saliva sample and chase buffer were premixed and the LFA was introduced as a dipstick into the solution. This testing method did result in decreased sensitivity and could be used as a method to adjust the performance to the target cutoff.

In an effort to further reduce sensitivity, polystreptavidin concentration at the test line was decreased. This adjustment did also result in decreased sensitivity, but not down to the target cutoff. Although both cases did not decrease sensitivity enough to reach the desired performance, the potential to fine tune the current LFA around saliva and a change in sensitivity requirements currently are believed to be feasible.

A. Testing Saliva with Dipstick Format Used in Example 1 for Plasma Sample

Four different recombinant antigen levels from saliva samples were tested.
Assembled 80 mm LFA Strips: CN95 Striped 1 μL/cm with 0.1 mg/mL Polystreptavidin+2% Sucrose as the test line, 0.3 mg/mL GAM as the control line; Ahlstrom Grade 6614 pad sprayed with 0.1% 400 nm Red Latex 40:1 Anti-hVEGFR DuoSet Capture Conjugate at 10.5 μL/cm, further sprayed with 22.5 μg/mL DuoSet Detection Antibody Solution at 6 μL/cm; Ahlstrom Grade 1281 pad was blocked with 1M MgCl₂ and 0.5% PLURONIC F127; and a 22 mm Ahlstrom Grade 243 pad as a wick.
300 ng/mL hVEGF R1 Standard
Saliva Sample Donor 1; Saliva Sample Donor 2; and Saliva Sample Donor 3
1×PBS with 0.75% PLURONIC F127
Borosilicate Glass Vials

Procedures:

### 1. Prepared Spiked Saliva Samples:

Spiked 8 μL of 300 ng/mL hVEGF R1 standard into 40 μL of select saliva sample and mixed to generate 60 ng/mL sample.
Spiked 4 μL of 300 ng/mL hVEGF R1 standard into 40 μL of select saliva sample and mixed to generate 35 ng/mL sample.
Spiked 1.3 μL of 300 ng/mL hVEGF R1 standard into 40 μL of select saliva sample and mixed to generate 35 ng/mL sample.

### 2. General Test Procedure:

Pipetted 100 μL of chase buffer (0.75% PLURONIC F127 in 1×PBS) into a borosilicate glass tube.
Pipetted 15 μL of designated sample onto the sample pad of a LFA strip.
Allowed for 30 seconds of strip development/sample absorption.
Dropped the prepared strips into the designated glass tubes (containing chase) so that the sample pad was in direct contact with the chase buffer.
Allowed for 14.5 minutes of strip development (15 minutes total), or another time as noted.
Removed strips from the tubes and placed onto a backing.
Took an image of the strips.
Allowed for an addition 15 minutes of development (30 minutes total).
Took an image of the strips.

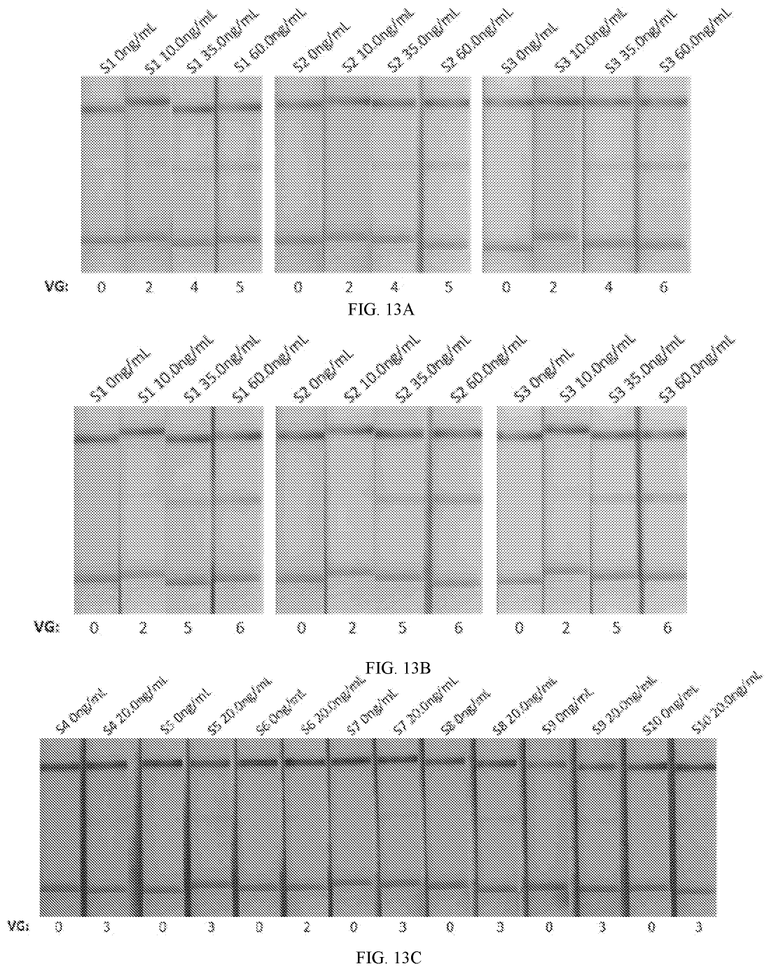

FIGS. 13A and 13B show the imaging results from full strips run with 15 μL of spiked and unspiked saliva samples that were developed for 15 minutes and 30 minutes, respectively.

The current experiment was designed to determine how a saliva matrix may perform when compared to plasma, which had been used in the majority of development to this point, and if the currently designed LFA had the potential to accommodate the change in matrix. Results, from the three freshly collected and spiked samples, show strong potential for the current LFA to accurately run saliva matrix based on test line intensity comparing very well to the plasma matrix. All three samples showed no visible test line for the unspiked saliva suggesting that specificity was acceptable even with the observably slower flow of the saliva sample when compared to the flow of plasma. B. Testing additional saliva samples from an increased number of donors at two different recombinant antigen levels.
Assembled 80 mm LFA Strips: CN95 Striped 1 μL/cm with 0.1 mg/mL Polystreptavidin+2% Sucrose as the test line, 0.3 mg/mL GAM as the control line; Ahlstrom Grade 6614 pad sprayed with 0.1% 400 nm Red Latex 40:1 Anti-hVEGFR DuoSet Capture Conjugate at 10.5 μL/cm, further sprayed with 22.5 μg/mL DuoSet Detection Antibody Solution at 6 μL/cm; Ahlstrom Grade 1281 pad was blocked with 1M MgCl₂ and 0.5% PLURONIC F127; and a 22 mm Ahlstrom Grade 243 pad as a wick.
300 ng/mL hVEGF R1 Standard
Saliva Sample Donor 4; Saliva Sample Donor 5; Saliva Sample Donor 6; Saliva Sample Donor 7; Saliva Sample Donor 8; Saliva Sample Donor 9; Saliva Sample Donor 10;
1×PBS with 0.75% PLURONIC F127
Borosilicate Glass Vials

Procedures:

### 1. Prepared Spiked Saliva Samples:

Spiked 2.7 μL of 300 ng/mL hVEGF R1 standard into 40 μL of select saliva sample and mixed to generate ˜20 ng/mL sample.

### 2. General Test Procedure:

Pipetted 100 μL of chase buffer (0.75% PLURONIC F127 in 1×PBS) into a borosilicate glass tube.
Pipetted 15 μL of designated sample onto the sample pad of a LFA strip.
Allowed for 30 seconds of strip development/sample absorption.
Dropped the prepared strips into the designated glass tubes (containing chase) so that the sample pad was in direct contact with the chase buffer.
Allowed for 14.5 minutes of strip development (15 minutes total).
Removed strips from the tubes and placed onto a backing.
Took an image of the strips.

Results:

FIG. 13C shows the imaging results from full strips run with 15 μL of spiked and unspiked saliva samples. CN95 membrane.

The current experiment was designed to expand the number of donors in order to further examine sample/matrix variability. Results indicate little to no difference in assay performance for 6 out of the 7 samples tested in the current experiment and that these 6 match up to the first 3 tested as well with regards to overall performance characteristics. Sample 9 was the only sample that did show observable differences, specifically with regards to a noticeably slower flow, but still generated a clean background and positive test line when provided an additional 5 minutes of development time.

### C. Quality Control and Fresh Versus Frozen-Thawed Saliva

Assembled 80 mm LFA Strips: CN95 Striped 1 μL/cm with 0.1 mg/mL Polystreptavidin+2% Sucrose as the test line, 0.3 mg/mL GAM as the control line; Ahlstrom Grade 6614 pad sprayed with 0.1% 400 nm Red Latex 40:1 Anti-hVEGFR DuoSet Capture Conjugate at 10.5 μL/cm, further sprayed with 22.5 μg/mL DuoSet Detection Antibody Solution at 6 μL/cm; Ahlstrom Grade 1281 pad was blocked with 1M MgCl₂ and 0.5% PLURONIC F127; and a 22 mm Ahlstrom Grade 243 pad as a wick.
CN95 Striped 1.0 μL/cm with 0.1 mg/mL Polystreptavidin+2% Sucrose TL, 0.3 mg/mL GAM CL.
Ahlstrom Grade 6614 Sprayed with 0.1% 400 nm Red Latex 40:1 Anti-hVEGFR DuoSet Capture Conjugate at 10.5 μL/cm, Sprayed with 22.5 μg/mL DuoSet Detection Antibody Solution at 6 μL/cm
Ahlstrom Grade 1281 Blocked with 1M MgCl₂ and 0.5% PLURONIC F127
Ahlstrom Grade 243
300 ng/mL hVEGF R1 Standard
Saliva Sample Donor 4; Saliva Sample Donor 6; Saliva Sample Donor 9; Saliva Sample Donor 10;
1×PBS with 0.75% PLURONIC F127
Borosilicate Glass Vials

Procedures:

1. Assembled Strips on 80 mm Backing Card with Sample Pad:
Placed membrane on 80 mm backing card positioned 20 mm from bottom edge.
Cut Ahlstrom Grade 243 pad to 22 mm and placed to overlap membrane by 2 mm.
Placed precut and sprayed Ahlstrom Grade 6614 to overlap membrane by 2 mm.
Placed cut and blocked Ahlstrom Grade 1281 pad to overlap conjugate pad by 2 mm.

### 2. Prepared Spiked Saliva Samples:

Spiked 2.7 μL of 300 ng/mL hVEGF R1 standard into 40 μL of select saliva sample and mixed to generate ˜20 ng/mL sample.

### 3. General Test Procedure:

Pipetted 100 μL of chase buffer (0.75% PLURONIC F127 in 1×PBS) into a borosilicate glass tube.
Pipetted 15 μL of designated sample onto the sample pad of a LFA strip.
Allowed for 30 seconds of strip development/sample absorption.
Dropped the prepared strips into the designated glass tubes (containing chase) so that the sample pad was in direct contact with the chase buffer.
Allowed for 14.5 minutes of strip development (15 minutes total).
Removed strips from the tubes and placed onto a backing.
Took an image of the strips.
Allowed for an addition 15 minutes of development (30 minutes total).
Took an image of the strips.

Results:

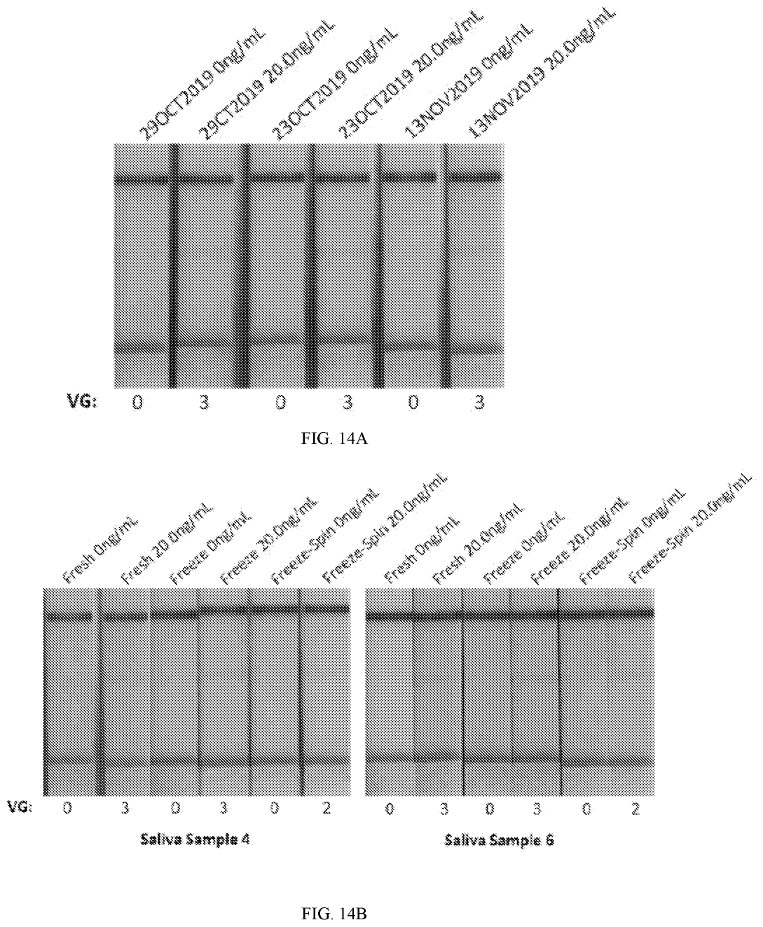

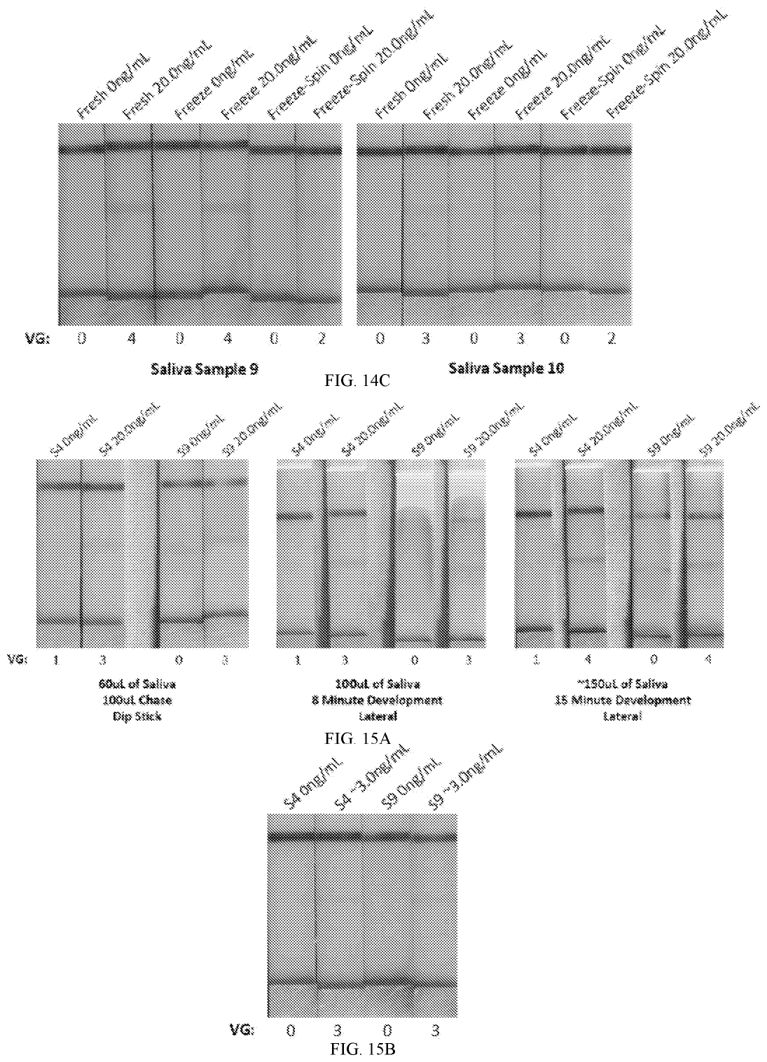

FIG. 14A shows results from full strips run with 15 μL of saliva sample 4. Comparing 3 preparations of strips containing different lots of material.

FIG. 14B shows results from full strips run with 15 μL of saliva samples 4 and 6 that were thawed after frozen. ‘Freeze’ involves a freeze thaw cycle of selected sample, after thaw sample was vortexed to ensure uniformity. ‘Freeze-Spin’ involves a freeze thaw cycle, the sample was vortexed, aliquoted, spiked as designed, and centrifuged at 10,000×g for 5 minutes.

FIG. 14C shows results from full strips run with 15 μL of saliva samples 9 and 10 that were thawed after frozen. ‘Freeze’ involves a freeze thaw cycle of selected sample, after thaw sample was vortexed to ensure uniformity. ‘Freeze-Spin’ involves a freeze thaw cycle, the sample was vortexed, aliquoted, spiked as designed, and centrifuged at 10,000×g for 5 minutes.

The current experiment was designed to examine the effect of a single freeze thaw on select saliva samples. First, LFAs were assembled from freshly prepared membrane and conjugate pad and compared against previously generated materials/LFAs. Three different strip/material lot combinations were quickly compared and showed strong consistency with regards to negative and positive test line formation as well as background clear and conjugate release.

With the newly prepared LFAs performing appropriately, four saliva samples were selected based on previous performance. These samples were removed from −20° C. storage from previous aliquot preparation as well as from 4° C. storage. −20° C. samples were allowed to thaw; the samples were then vortexed to ensure uniformity and aliquoted into separate tubes. The tubes designed to be centrifuged were spiked with antigen before centrifugation; they were then mixed and centrifuged at 10,000×g for 5 minutes.

Results comparing the 4° C. stored saliva with thawed samples and thawed samples that had been spun showed some difference in line intensity specifically with the centrifuged samples; however, this may be a result of the centrifuge conditions being too strong for the antigen resulting in an amount of antigen pelleting down along with the mucus. It should be noted that after 15 minutes the development of the strips appear overall similar as pictured above; however, based on visual observations during testing it was concluded that the thawed samples were noticeably more turbid than the 4° C. samples and that the thawed samples that were not centrifuged flowed noticeably slower than the other two preparations.

D. Evaluation of Increased Saliva Volume and Using Characterized Plasma to Spike into Saliva
Assembled 80 mm LFA Strips: CN95 Striped 1 μL/cm with 0.1 mg/mL Polystreptavidin+2% Sucrose as the test line, 0.3 mg/mL GAM as the control line; Ahlstrom Grade 6614 pad sprayed with 0.1% 400 nm Red Latex 40:1 Anti-hVEGFR DuoSet Capture Conjugate at 10.5 μL/cm, further sprayed with 22.5 μg/mL DuoSet Detection Antibody Solution at 6 μL/cm; Ahlstrom Grade 1281 pad was blocked with 1M MgCl₂ and 0.5% PLURONIC F127; and a 22 mm Ahlstrom Grade 243 pad as a wick.
300 ng/mL hVEGF R1 Standard
Saliva Sample Donor 4; Saliva Sample Donor 9;
Plasma Sample 99 R-d3
1×PBS with 0.75% PLURONIC F127
Borosilicate Glass Vials

Procedures:

### 1. Prepared Spiked Saliva Sample (Plasma):

Spiked 40 μL of select saliva with 5 μL of plasma. Mixed via vortex.
Spiked 15 μL of select saliva with 5 μL of plasma. Mixed via vortex.

### 2. General Test Procedure:

Pipetted 100 μL of chase buffer (0.75% PLURONIC F127 in 1×PBS) into a borosilicate glass tube.
Pipetted designated sample onto the sample pad of a LFA strip.
Allowed for 30 seconds of strip development/sample absorption.
Dropped the prepared strips into the designated glass tubes (containing chase) so that the sample pad was in direct contact with the chase buffer.
Allowed for 14.5 minutes of strip development (15 minutes total).
Removed strips from the tubes and placed onto a backing.
Took an image of the strips.

### 3. General Lateral Test Procedure:

Pipetted designated sample onto the sample pad of a LFA strip.
Allowed for 15 minutes of strip development (15 minutes total).
Placed onto a backing.
Took an image of the strips.

Results:

FIG. 15A results from full strips run with saliva samples. 60 μL of saliva was chased with 100 μL of chase buffer and run as a dipstick in the first 4 LFAs shown above. The next four involved direct injection of sample onto the sample pad without chase and allowed to develop flat on the benchtop (lateral flow immunoassay format). Flow halted for sample 9 at or around 7 minutes. In order to examine if this flow would resume, more sample was pipetted onto the sample pad of each strip totaling around 150 μL. The line that appears in the 0 ng/mL sample 9 is due to flooding that occurred upon addition sample injection.

FIG. 15B shows results from full strips run with 15 μL of spiked and unspiked saliva samples. Saliva was spiked with a characterized plasma sample with a determined concentration of 23.96 ng/mL in order to reach an estimated 3 ng/mL level in the saliva samples.

The current experiment was designed to examine the effect of increasing saliva volume in the system as well as attempt to determine with higher confidence the current sensitivity of the LFA using native protein spiked into select saliva samples. The first four LFAs tested used 60 μL of saliva and 100 μL of chase as previously performed as a dip stick test. Results indicate a slight increase in assay sensitivity but the strong flowing Sample 4 showed overall similar performance as the previously tested 154, sample volume. Sample 9, which is known to have significantly slower flow, did show a dramatic reduction in background clearance resulting in a difficult to read test line at 15 minutes.

Next 100 μL of saliva sample was directly injected onto the sample pad and allowed to run in a lateral format, due to the concern of decreased flow, and allowed to develop without any added chase buffer. For Sample 4 performance was noticeably different than when using a chase but the difference was expected and still allowed for easily readable test and control lines. For Sample 9 the LFA completed halted flow after 7-8 minutes of development as shown above; at this point more sample was introduced on the sample pad to determine if additional liquid would restart the system flow. This extra addition of sample did restart flow but due to the unknown ability of the LFA to handle additional volume the system flooded resulting in the abnormal ‘line’ of conjugate that appears in the image. It is believed that with further examination of volume capacity that flooding can be avoided.

Last, the two saliva samples were spiked with plasma sample 99 R-d3, which had been previously characterized and tested with the current LFA. An estimated 3 ng/mL saliva sample produces strongly visible lines.

### E. Testing Pre-Diluting Sample in Chase Buffer Before Strip Introduction and Testing Lower Polystreptavidin Test Line Concentration

Assembled 80 mm LFA Strips: CN95 Striped 1 μL/cm with 0.1 mg/mL Polystreptavidin+2% Sucrose as the test line, 0.3 mg/mL GAM as the control line; Ahlstrom Grade 6614 pad sprayed with 0.1% 400 nm Red Latex 40:1 Anti-hVEGFR DuoSet Capture Conjugate at 10.5 μL/cm, further sprayed with 22.5 μg/mL DuoSet Detection Antibody Solution at 6 μL/cm; Ahlstrom Grade 1281 pad was blocked with 1M MgCl₂ and 0.5% PLURONIC F127; and a 22 mm Ahlstrom Grade 243 pad as a wick.
CN95 Striped 1.0 μL/cm with 0.05 mg/mL Polystreptavidin+2% Sucrose TL, 0.3 mg/mL GAM CL.
Ahlstrom Grade 6614 Sprayed with 0.1% 400 nm Red Latex 40:1 Anti-hVEGFR DuoSet Capture Conjugate at 10.5 μL/cm, Sprayed with 22.5 μg/mL DuoSet Detection Antibody Solution at 6 μL/cm
Ahlstrom Grade 1281 Blocked with 1M MgCl₂ and 0.5% PLURONIC F127
Ahlstrom Grade 243
Saliva Sample Donor 4; Saliva Sample Donor 9;
Plasma Sample 99 R-d3
1×PBS with 0.75% PLURONIC F127
Borosilicate Glass Vials

Procedures:

### 1. Prepared Spiked Saliva Sample (Recombinant):

Spiked 200 μL of saliva with 13.330_, of 300 ng/mL hVEGF R1 Standard. Mixed via vortex.

### 2. Prepared Spiked Saliva Sample (Plasma):

Spiked 20 μL of saliva with 2.5 μL of plasma. Mixed via vortex.

### 3. General Test Procedure:

Pipetted 100 μL of chase buffer (0.75% PLURONIC F127 in 1×PBS) into a borosilicate glass tube.
Pipetted 15 μL of designated sample onto the sample pad of a LFA strip.
Allowed for 30 seconds of strip development/sample absorption.
Dropped the prepared strips into the designated glass tubes (containing chase) so that the sample pad was in direct contact with the chase buffer.
Allowed for 14.5 minutes of strip development (15 minutes total).
Removed strips from the tubes and placed onto a backing.
Took an image of the strips.
Allowed for an addition 15 minutes of development (30 minutes total).
Took an image of the strips.

Results

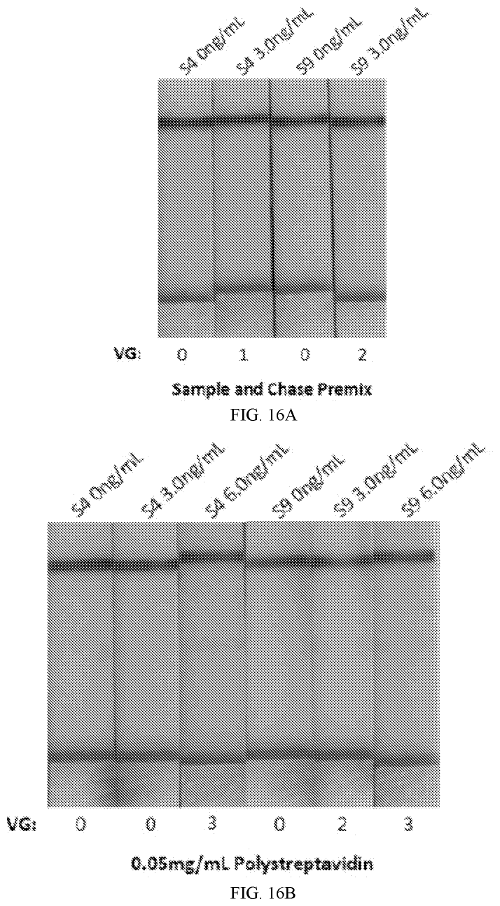

FIG. 16A shows results from full strips run with 15 μL of saliva samples that were prediluted in chase buffer. CN95 membrane. 0.1% DuoSet Capture Latex. 22.5 ug/mL DuoSet Detection Solution. 0.1 mg/mL Polystreptavidin test line concentration. Spiked samples used plasma sample 99 R-d3.

FIG. 16B shows results from full strips run with 15 μL of saliva samples and where the polystreptavidin concentration was lowered. CN95 membrane. 0.1% DuoSet Capture Latex. 22.5 ug/mL DuoSet Detection Solution. 0.05 mg/mL Polystreptavidin test line concentration. Spiked samples used plasma sample 99 R-d3.

The current experiment was designed to examine the effect of diluting the sample in the chase buffer and the effect of lowering polystreptavidin concentration on the LFA's sensitivity. Results from the premix of 100 μL of chase buffer and 15 μL of spiked (using characterized plasma) and unspiked saliva samples showed a slight decrease in sensitivity compared to the previous test format along with significantly improved flow and conjugate release; the LFA is this case appears to be too sensitive as an estimated 3 ng/mL concentration sample still provides a visible test line with the slower flowing saliva sample.

Results from testing membrane striped with a lower polystreptavidin concentration also showed a visible decrease in sensitivity; the LFA is this case appears to be too sensitive as an estimated 3 ng/mL concentration sample still provides a visible test line with the slower flowing saliva sample.

As such, another embodiment is to combine lowered test line concentration of polystreptavidin with pre-diluting sample in chase buffer in the device and assay methods.

### Example 11. Further Studies

Development work continued by first examining methods to manipulate the sensitivity of the assay by testing combinations of the following changes: adjusting polystreptavidin concentration (including 0.1, 0.05, 0.025 or 0.01 of polystreptavidin tested with 3.0 ng/mL or 6.0 ng/mL or no sample), switching from a dip stick to a lateral (run horizontally) test, and applying sample without a chase. Results indicate that a combination of decreasing polystretpavidin concentration and changing the assay to a lateral format provides a pathway to make very controlled adjustments to sensitivity. Results indicate that the decrease in test line concentration continues to have a dramatic effect on sensitivity resulting in a loss of detection when the polystreptavidin concentration dropped below 0.05 mg/mL for a 3 ng/mL sample. Based on this result, 0.025 mg/mL and 0.01 mg/mL polystreptavidin concentrations were tested once again but with 6 ng/mL spiked saliva samples. Results from this test showed no visual test line indicating that the decreased polystreptavidin has raised the limit of detection to above 6 ng/mL. Knowing that 0.025 mg/mL could not pick up a 6 ng/mL sample in the dip stick format. The assay was run laterally, placed flat on the bench, with saliva directly in order to provide an overall decrease in flow which has been shown to increase sensitivity previously. Results suggest a slight increase in sensitivity but still do not provide a strongly visible line at 6 ng/mL. Lastly knowing that the lateral format significantly increases flow and thereby decreases sensitivity, the 0.05 ng/mL polystreptavidin concentration was tested as a lateral assay with a 6 ng/mL sample. The results from this test show a visible line at 6 ng/mL that was similar to the 3 ng/mL intensity when the assay was a dipstick: this result suggests that a switch in assay format may provide the best means for fine tuning of assay sensitivity.

Next, testing moved into a more in-depth examination of a saliva only test: no chase buffer needed.

A number of saliva samples were tested to determine volume requirements, variations in flow, and metering requirements. In samples ID S5-S7, minimum volume required for full sample clear is 112.5 μL, maximum volume that avoids flooding is 125 μL and the time required to apply sample at maximum volume is 1.5 minutes; in sample ID S8, minimum volume required for full sample clear is 120 μL, maximum volume that avoids flooding is 125 μL and the time required to apply sample at maximum volume is 1.5 minutes; and in sample ID S9 (more viscous sample), minimum volume required for full sample clear is 130 μL, maximum volume that avoids flooding is 135 μL, and the time required to apply sample at maximum volume is 2.5 minutes. Results support a number of key findings: minimum and maximum volume requirements vary depending on the sample, time required to inject said volumes differs depending on the sample, viscous samples (such as sample 9) will continue to be exceptionally difficult to run directly with the currently designed LFA in order to balance volume requirements while avoiding flooding of the LFA.

Testing of the impact of a freeze-thaw cycle of samples was repeated with a focus on how the antigen is affected when in the frozen and thawed saliva matrix. Freeze thaw does appear to impact the sensitivity in both samples tested. They show a general decrease in line intensity when comparing fresh versus frozen samples. Results also suggest that this decrease in sensitivity differs from sample to sample, as sample 9 showed less decrease in test line intensity than sample 4. Spinning the sample to pellet the mucin did not uniformly decrease assay sensitivity, as sample 4 showed little to no decrease while sample 9 did show a decrease. Given that there was a decrease in test line intensity in both samples from a freeze/thaw cycle, it is likely that other saliva samples will also show a visual grade decrease when frozen and thawed; this decrease varies from sample to sample and may also be enhanced with more cycles or longer time stored frozen. Results indicate that the antigen does appear to be negatively impacted due to a freeze-thaw cycle by producing a less intense test line than seen with a fresh, never frozen sample. Additionally, this effect appears to be variable based on different individual samples.

Six saliva samples were tested using the plasma LFA. Results showed no visually significant test lines for any of the tested samples: although; P0024 and P0029 showed a partially developed test line initially. A retest of both samples showed no visual detected test line. Due to the lack of a visual test line in all cases, P0025 (the slowest flowing) and P0027 (the fastest flowing) were spiked and tested once again to determine if the matrix itself was inhibiting signal. The results suggest that this was not the case as both samples produced test lines with expected intensity based on previous testing at the 10 ng/mL level. Overall, results showed that all six samples have low sFlt-1 values, with all six samples showing no visible test line formation.

Last, an experiment was performed that examined sample pad material, membrane, and cassette effects in order to provide more consistent flow with a saliva only sample. Change in sample pad material (testing untreated Ahlstrom Grade 1660, 1662, 1663 and 319 materials) provided a significant improvement in metering of sample with both sample 9-2 and sample 7 being able to tolerate the addition of 130 μL of saliva without flooding; these two pads also appeared to filter a significant amount of mucin out of the sample. This may provide advantages with regards to sample to sample variability. Given the improvement in metering that Ahlstrom Grade 1662 and 1663 materials provided, samples were spiked and tested with the untreated pads against the currently developed LFA. All four untreated tested pads showed little to no signal at 10 ng/mL while the control strip generated a strongly visible test line. This result begs the question whether this is a result of the antigen getting trapped in the sample pad or of the pad treatment responsible for creating a binding environment. Ahlstrom Grade 1662 and 1663 materials will be blocked and the test will be rerun. MgCl₂ and PLORONIC F127 treatment is a very important element of the LFA system, as the tested level showed little to no test line intensity without the treatment but with the treatment a visible test line appears, albeit a visual grade below the LFA. Based on these results, varying concentrations of the sample pad treatment buffer should be prepared and tested using the 1662 and 1663 pads. Swapping CN95 with membranes characterized to having larger pore sizes, which results in faster flow, showed little to no improvement with regards to flooding: MDI-15 μm did show some improvement with the faster flowing sample 6, but did not show improvement with sample 9-2. Lastly, a generic cassette (FIG. 17) was used with the current LFA. Results suggest that a cassette may allow the current sample pad to be used if a custom cassette was created with a sample well designed to meter the release of the sample into the pad slowly. Results from the testing highlight sample pad material and cassette design/implementation to be potential pathways for improvement of a saliva only system by helping with flow issues and reducing aggregation. Initial testing with the pads also showed the loss of a dose response but was later determined to be more strongly related to the necessity of the sample pad treatment.

These results indicate the limit of detection (LOD) for saliva specimens may be 5-10 times lower than that for plasma specimen. For the time being, the new target is 2.5 ng/mL rather than 5 ng/mL.

### Example 12. Additional Studies

Development work continued by first going into more depth regarding sample pad material and sample pad treatment buffer concentration. Results from testing with the Ahlstrom Grade 1662 and 1663 pad in comparison to the currently used 1281 pad suggest that the current buffer (which is sample buffer of 0.5M-1.5M MgCl₂ plus 0.25%-0.75% PLURONIC F127) used is likely at the optimal concentration for the assay as results from testing above and below the current concentration resulted in a loss of sensitivity in both cases. Results also support the previous conclusion that the 1662 and 1663 pad material itself is lending to a reduction in sensitivity.

Next, testing moved into setting up an initial stability study. The baseline, T0, testing was performed with three pooled plasma samples and three pooled saliva samples. Initial results were as ‘expected’ based on previous testing at the levels of antigen prepared. T1 for the 45° C. accelerated stability test was also performed. Results from the T1 testing show little to no change from the TO test supporting an initial stability of up to 1.5 months.

Last, an experiment was performed that examined increasing sensitivity through an increase in test line concentration in combination with the sample pad material. Initial results show that a test line concentration of ≤0.25 mg/mL is likely as high as the current LFA can handle without generating significant nonspecific binding. Two polystreptavidin concentrations were selected to be compared with the currently used 0.1 mg/mL concentration: 0.25 mg/mL and 0.75 mg/mL. Additionally, 2 sample pads were both treated with 1M MgCl₂/0.5% Pluronic F127 sample buffer: Ahlstrom grade 1281 and Ahlstrom grade 1662. Initial results with the 1281 sample pad show expected trending, where an increase in test line concentration resulted in increasing test line intensity. The 0.25 mg/mL TL concentration appears to be on the border of generating NSB as negative samples appear to be starting to form a visible test line and the 0.75 mg/mL concentration appears to be too high as all negative samples show a significantly visible test line. Initial results with the 1662 sample pad show unexpected results: namely that negative and positive samples could not be differentiated. Since the 1662 pad had not been tested as a dipstick or with a chase previously, the results of initial testing with the pad begged the question of whether these results are due to format, the pad material, or both. Testing also showed that significant adjustments will need to be made if a switch from the 1281 pad to the 1662 pad were made for use in a dipstick or chase buffer assay format; the 1662 pad performs well in a direct saliva without chase and is required in order for the assay to differentiate appropriately between negative and positive samples. A last test was performed to evaluate the 1662 pad run horizontally on the benchtop as a lateral test using a chase, and with direct saliva, using two contrived samples. Results suggest that currently separation can be achieved with the 1662 pad when the assay is run with direct saliva.

Subsequently, development work continued by first going into more depth regarding polystreptavidin concentration with saliva samples focusing the concentration around 0.2 mg/mL. The current experiment was designed to test if a concentration of polystreptavidin could be found that increases positive sample test line intensity without increasing negative sample test line intensity. Results with both 0.15 and 0.2 mg/mL polystreptavidin concentrations do show a general trend with clinically assigned samples of separating positive and negative samples if a diagnostic cutoff of 2 ng/mL is used outside of sample P0028, which has consistently generated a test line even when a 0.1 mg/mL polystreptavidin test line was used to test the sample. Unfortunately, as polystreptavidin concentration is increased the test line for the negative sample is also increased resulting in separation similar to the lower concentration test lines. Results suggest that the concentration of 0.15 mg/mL may be the maximum concentration for polystreptavidin in order to avoid increasing signal to the point that negative and positive samples cannot be distinguished consistently with the currently developed LFA for saliva. Differentiation of the three sFlt-1 samples tested was achieved based on their assigned concentrations but comparing the signal of saliva valued at 3.5 ng/mL with saliva spiked using characterized plasma to 4 ng/mL shows a significant difference. There was a noticeable difference between a plasma spiked saliva sample and a pure saliva samples: the spiked sample assumed to be around 4 ng/mL based on the assigned value of the plasma used to spike the saliva is producing significantly stronger line formation than the assigned saliva sample at 3.5 ng/mL. This difference suggests that recognition and capture of sFlt-1 in saliva may be different due to how the antigen is presented in saliva, that the matrix itself is noticeably affecting the LFA system and capture of antigen, the freezing of the sample is significantly impacting availability of the antigen, or all of the above.

Testing moved to examine if sensitivity could be improved by increasing sample volume when using a chase buffer in the dip stick assay format. Initial results showed that as sample volume increased the performance seemingly decreased but this result depended on the individual samples. Further examination of this conclusion by directly comparing dip stick and lateral formats resulted in data that supports the conclusion: variability between saliva samples is more impactful on LFA performance than previous testing with plasma.

Last, stability testing continued with increased replicates and plasma only for strips stored at room temperature, 37° C., and 45° C. The current experiment was designed to test the second selected time point for the 45° C. accelerated stability portion, the first selected time point for the 37° C. accelerated stability portion, and the first selected time point for the room temperature stability portion of the current stability study. Results show a single visual grade fluctuation between replicates of a given sample and when compared to T0 but overall change from the baseline read is little to none for all three conditions supporting an accelerated equivalent stability of 3 months for the current plasma sample based LFA. Results from the 45° C. accelerated study support a stability of up to 3 months for the plasma LFA.

### Example 13

Development work continued by first screening a number of sample additives for pretreating a sample, selected on the basis that the effects of mucin and other saliva unique proteins would be reduced resulting in stronger recognition of saliva native sFlt-1. After 2 rounds of screening a total of 24 different conditions, tris (2-carboxyethyl) phosphine (TCEP) appears to provide the most improvement with regards to controlling flow variability between saliva samples; however, while TCEP improved flow variation it did not appear to improve native saliva sFlt-1 recognition as it was currently tested. The tested additives included 4M NaCl, 2.5M KCl, 10% BRIJ-3 5, 10% BRIJ-52, 10% BRIJ-98, 10% CHEMAL LA-9, 10% IGEPAL CA 630, 10% SDS, 8M UREA, 500 mM TCEP, 5% Polyethylene oxide MW 100,000, 2.5% polyethylene oxide MW 400,000, 5% polyvinylpyrollidone MW 360,000, and 1×PBS with 0.75% PLURONIC F127. Based on results with regards to test line intensity and system flow improvement the following additives were selected for testing with native unaltered saliva sample: 5 mM TCEP, 0.01% SDS, 200 mM UREA, 1.5% BRIJ-35, and 1% IGEPAL CA 630.

Next, testing moved to examine a third set of 6 saliva samples. Results of the third sample panel unfortunately do not provide separation between samples above and below 2 ng/mL similarly to the second panel. This result confirmed that a significant difference in the performance of native saliva samples compared to spiked samples (whether spiked with recombinant or plasma native sFlt-1) exists.

Following the third panel test, testing moved to examine how sample pad treatment, sample pad and conjugate pad, and incubation may alter the performance of the assay with the saliva matrix by going back to a wet system using half strips. Results suggested that MgCl₂ is still to be considered a very important component as sample variability is dramatically increased when it is removed from the system. Subsequent testing with the wet system also showed that while incubation may improve performance and variability between samples it does not necessarily improve recognition of native saliva sFlt-1 over spiked sFlt-1.

Additives were once again explored based on the previous screening to include a second reducing agent (Guanidine HCl, about 20 mM or 40 mM) and a chelating agent (EDTA, about 100 mM). Results from testing the third set of saliva samples show the reducing agent once again providing improved sample to sample variability in flow but once again it did not appear to improve native saliva sFlt-1 recognition as it was currently tested. The reducing agents may be negatively impacting the antibodies and reducing performance. The current experiment was designed to further optimize TCEP concentration, test a chelator in the system (EDTA), and test a second reducing agent (Guanidine HCl) in order to continue exploring pathways to improve patient sample recovery. Results from TCEP concentration testing suggest that the optimal concentration, in order to observe the positive effects of decreased sample variability with regards to flow, lies somewhere between 2 mM and 3 mM based on sample volume. EDTA does not appear to improve the system's performance based on obtained results, in fact it may decrease overall performance based on loss of signal in not only the test line but control line as well. Combining EDTA with TCEP also does not appear to improve performance based on results as the test line intensity, control line intensity, and flow time did not show improvement over TCEP alone. Guanidine HCl is another reduction agent similar to TCEP. Results from testing this in the system show a similar effect as TCEP although at a much higher concentration. Overall the performance results lie in between no additive and 2.5 mM TCEP for Guanidine HCl. A concentration of 15 mM was carried into testing with the third sample panel. Results are similar to those obtained when using TCEP: reduction of signal for samples P0005 and P0006 which were not expected to generate significant test lines without any increase in signal for sample P0001 and P0003 which were expected to generate a positive test line. While these reagents show a clear improvement in sample flow, they are likely negatively impacting the other assay reagents.

Last, stability testing continued with strips stored at room temperature, 37° C., and 45° C. Results from the 45° C. accelerated study support a stability of up to ˜9.5 months for the plasma LFA. The goal is to test samples with LFA devices incubated at 45° C. for 22 days (5.75 month equivalency) or 29 days (˜7.5 month equivalency) or 36 days (˜9.5 month equivalency), incubated at 37° C. for 22 days (2.5 month equivalency) or 29 days (˜7.5 month equivalency) or 36 days (˜9.5 month equivalency), and stored at room temperature for 22 days or 29 days or 36 days. Results show a single visual grade fluctuation between replicates of a given sample and when compared to T0, but overall change from the baseline read is little to none for all three conditions supporting an accelerated equivalent stability of ˜5.75 months for the current plasma sample based LFA. Results show a single visual grade fluctuation between replicates of a given sample and when compared to T0, but overall change from the baseline read is little (at most 1 visual grade) to none for all three conditions supporting an accelerated equivalent stability of ˜7.5 months for the current plasma sample based LFA. Results show a single visual grade fluctuation between replicates of a given sample and when compared to T0, but overall change from the baseline read is little (at most 1 visual grade) to none for all three conditions supporting an accelerated equivalent stability of ˜9.5 months for the current plasma sample based LFA.

### Example 14

Development work continued by first increasing assay ‘sensitivity’ by dramatically increasing polystreptavidin concentration, testing biotin-ab/latex addition order, testing increased amounts of biotin-ab/latex conjugate solutions, and testing adjusted ratios of biotin-ab and latex conjugate solutions. The increase in test line concentration resulted in visible test line formation with an assumed ‘negative’ saliva sample, possibly due to recognition of endogenous sFLT-1; however, increases to biotin-ab and latex conjugate solution and adjustment of the ratio of these two reagents did not further increase test line intensity with the increased polystreptavidin test line concentration. Specifically, the current experiment was designed to increase sensitivity of the assay as much as possible using an assumed ‘negative’ sample generating a test line as a benchmark. Initial testing looked at volume, biotin-ab/latex addition order, and sample diluent. For the current 60 mm strip it was evident after the first strips were tested that approximately 50 μL of total sample/diluent volume was the maximum volume possible to avoid the flooding. Addition ordered appeared to make little to no difference with regards to background or test line intensity. Sample diluent clearly requires MgCl₂ in order to provide proper conjugate flow; without the addition of MgCl₂ the conjugate aggregates and does not flow up the membrane. Next, Biotin-Ab to Latex ratio and volumes were adjusted and tested. Results may suggest that decreasing the ratio between the two solutions by increasing the biotin-ab portion may provide slightly better conjugate clearing. Results also support a maximum latex conjugate volume of approximately 6.75 μL before the assay begins showing diminishing gains via increased background without increased line intensity.

Last using the increased biotin-ab/latex amounts, 1 mg/mL polystreptavidin test line, and a 25/25 sample dilution ratio with sample buffer as the diluent, the third sample panel was tested. Results do show a stronger line intensity for P0001 than previously obtained; however, the correlation of determined sFlt-1 concentration to line intensity remains low.

Next, testing moved to examine pre-dilution of saliva sample buffer addition to the LFA strip. Overall, results indicate that a lateral format is much more consistent as well as provide better performance whether a pre-dilution step is included or not. Results also do not show any significant benefit to pre-dilution of sample instead suggesting that increasing sample volume is not only possible in the lateral format but that it can be increased without negatively impacting assay performance to a certain extent. Specifically, the current experiment was designed to further examine sample volume and sample pre-dilution. Initial results examining pre-dilution of sample shows a significant decline in assay performance when the assay is run as a dipstick; however, when the assay format was moved to a lateral format the performance greatly improved. This result, along with previous testing of neat saliva testing, strongly supports that saliva should be run laterally regardless of sample volume. Based on the results from initial testing, specifically the odd line formation of the highly dilute sample not previously observed, chase buffer composition was examined. Results are limited but suggest that removal of the 1×PBS from the chase buffer may provide mild assay performance improvement by providing more uniform line formation. Next, the ratio of sample to sample diluent was examined. Results indicate that with pre-dilution sample volume can be increased for the tested sample up to a volume of 75 μL. Further testing revealed that adding TCEP to the sample solution allows this higher sample volume to flow appropriately through CN140 and be increased to 100 μL with CN95 and flow appropriately. This increase of sample volume without diminishing flow rate may be achievable through sample treatment outside of TCEP such as MgCl₂ and PLURONIC concentration increases.

This adjustment in maximum sample volume was explored in more depth by adjusting dilution ratios, diluent solution, and sample additives. Overall results from multiple experiments show that maximum volume is dependent on the donor, but even for a ‘difficult’ donor significant increase in sample volume (from the previously chosen 15 uL) can be achieved without negative effect by either including a minimum concentration of MgCl₂/Pluronic F127 or TCEP. Specifically, the current experiment was designed to further examine sample volume and diluent ratio using a different saliva donor while also exploring if pad material could alter assay performance by testing a glass fiber conjugate pad next to the currently used polyester conjugate pad. Results from the pad testing do show that the glass fiber pad generated more conjugate aggregate at the pad/membrane interface; however, overall assay performance remains similar if not identical. Further testing of sample volume to diluent ratio suggest that an 80 μL sample volume to 20 μL sample diluent may be possible without reducing assay performance as the ratio of 90 μL sample volume to 10 μL of diluent shows a dramatic decrease in line intensity. This may be a result of increased sample volume or a result of decreased MgCl₂/PLURONIC F127 in the assay system. In a subsequent testing of the maximum sample volume using a fresh, more viscous, saliva sample, previously used ratio of 80/20 was not able to flow appropriately resulting in very weak test and control lines; this was also demonstrated by adjusting the ratio to a much lower sample volume of 50/50 producing lines that were expected based on previous results. Further testing of these volumes and treatments show that treatment of sample with an appropriate amount of MgCl₂/PLURONIC or TCEP is crucial to achieving flow with more viscous saliva sample if sample volume is to be increased.

Lastly, freeze thaw effect was briefly examined with one sample donor. One and two freeze thaw cycles (−20° C. to room temperature) were performed with a single sample aliquot and tested next to the same donor sample stored at 4° C. Results show little to no difference in test line formation; however, there may be other reasons for this and it cannot be concluded that freeze thaw cycles do not decrease recovery of endogenous saliva sFlt-1. It is possible that the test line signal observed from the presumed negative sample is not due to specific binding and rather is non-specific binding that is not impacted by the freeze thaw.

### Example 15

Development work continued by first recording the last planned time point for the plasma LFA stability study. Accelerated study results indicate that the current assay will be stable for up to ˜1 year as performance shifts from test point to test point were all within acceptable ranges (+/−1 visual grade from TO).

Next, testing moved to examine endogenous sFlt-1 recognition in saliva by performing a depletion study. The goal of the study is to deplete endogenous sFlt-1 from in-house derived saliva sample through using two methodologies: DuoSet Capture Conjugated Latex and DuoSet Capture Antibody. Results from depleting unaltered sample with both latex conjugate and free antibody support the conclusion that endogenous sFlt-1 in saliva is being recognized by the current LFA as depletion of the sample with project related antibody showed stronger reduction in test line signal than reduction of signal with unrelated conjugate and free antibody. Specifically, the current experiment was designed to investigate recognition of endogenous sFlt-1 using the current ‘high sensitive’ LFA by theoretically depleting the sample and testing it against unaltered sample. Results support the conclusion that the assay is recognizing native/endogenous sFlt-1 based on the fact that depletion of the sample using conjugated latex or antibody results in a reduction of visual grade consistently. The small change in visual grade can be explained by the expected concentration of a negative saliva sample residing in the sub 1 ng/mL concentration level (≤6 pM-17 pM depending on isoform). A second conclusion that can be made from this experiment is that generic antibody may reduce what appears to be nonspecific binding. This is demonstrated by the fact that the unrelated antibody addition appears to reduce the test line intensity when compared to the unaltered sample; this reduction is then decreased further with the addition of the project related antibody again suggesting that endogenous sFlt-1 is being removed.

Results from the depletion study led into examination of multiple IgG sources as a block for observed NSB. Results from multiple rounds of testing show that goat IgG has a strong potential to decrease NSB in certain samples. Specifically, the addition of IgG as a sample additive does have a dramatic effect on assay performance. This is based on increase and decrease of test line intensity depending on the species of IgG used. The effect of these IgGs is dependent on the donor as results show a significant difference in effect depending on the donor with regards to line intensity, conjugate flow, and overall aggregation. Goat IgG appears to provide the best overall performance especially with regards to sample 12; Mouse IgG also provides significant reduction but given that it is similar to the goat IgG with regards to NSB reduction and that the control system would have to be significantly adjusted if Mouse IgG was included in the sample buffer the goat IgG would be preferred.

Lastly, MgCl₂ concentration was further explored through direct addition of MgCl₂ (to avoid sample dilution) and in a dried down system in order to find an optimal concentration of the salt that would allow for increased saliva sample volume without a decrease in assay performance. Initial testing with the solid MgCl₂ showed promise to increase even the ‘poor flowing’ sample volume to 1004, without a loss of flow which was seen previously; however, attempts to move into a dried system did not produce results that show improvement over the currently used 1M concentration of MgCl₂ in the sample buffer. The current experiment was designed to test if increased salt and detergent concentration in the sample pad would allow for increased sample volume (while removing any chase) to flow as desired with little to no negative effect with regards to background and aggregation. Results indicate that a ‘sweet’ spot for MgCl₂ and PLURONIC likely exists as too much decreases assay performance with specific samples, as shown above when a 2M MgCl₂ concentration was used for sample 13, while too little also decreases performance to the level that sample volume must be decreased for proper flow to be achieved as shown in previous experiments. Based on the current results, more concentrations of MgCl₂ can be tested to find the maximum concentration possible before assay performance is negatively affected for given samples; if this concentration does not sufficiently improve flow of the larger sample volume, the inclusion of TCEP in the sample buffer solution at an appropriate concentration can be explored. Goat IgG was once again tested, this time with a full strip and dried reagents. Results support those obtained previously that conclude some samples show a significant reduction in NSB when goat IgG is included.

### Example 16

Development work continued by first testing TCEP integration into the dried down LFA system. Results from testing showed significant improvement in flow and aggregation as concentration of TCEP in the sample buffer was increased, although how dramatic the effect was appeared sample dependent. Specifically, the current experiment was designed to test how TCEP would perform if it was included in the sample buffer and dried into the sample pad along with the other components. Results show a clear trend with both of the selected samples: typically slower flowing S13 and faster flowing S12, as TCEP concentration in the buffer is increased the flow of the LFA system is improved. For sample 12 the aggregation that appears between the conjugate/membrane interface is all significantly reduced once the TCEP concentration reaches the 0.5 mM level.

Next, testing moved to an in-house screen of freshly obtained saliva samples. Results showed significant variability in sample performance with regards to flow, aggregation, and test line formation. Testing also resulted in two samples that could not flow through to the wick without the addition of more sample. Specifically, the current experiment was designed to explore sample variability with the current assay format and components as well as perform a quick QC check on freshly prepared latex conjugate. Results from initial testing show significant variability between samples with regards to both flow, aggregation, and test line formation. Samples 14 and 16 showed significant reduction of flow to the point where flow completely stopped and would not have continued without the addition of 15 and 30 of sample respectively. Sample 15 (same donor as sample 12 and sample 4) continued to produce a strong test line as it had previously. This test line has been dramatically reduced in previous testing via inclusion of goat IgG in the test system suggesting that the test line formed is mostly NSB and that it likely could be reduced with IgG inclusion. Dose response testing using recombinant antigen showed expected differences between unspiked and spiked saliva samples. The test also demonstrated that older and freshly prepared conjugate solutions were performing very similarly. Lastly, samples 14 and 16 were tested with preassembled strips that had a sample pad containing TCEP in order to determine if flow could be improved; however, noticeable improvement was limited with regards to flow and no difference is observable with regards to aggregation. The only significant improvement determined was that additional sample was not necessary in order to get the sample solution to reach the strip's wick. This difference in result from previous testing may suggest that component effectiveness is closely tied to individual samples and that the component does not necessarily provide a blanketed improvement effect on all saliva samples.

Results from the testing of in-house samples led to further examination into possible methods to reduce observed variability in fresh samples. Methods revolved around increase in MgCl₂, PLURONIC F127, and TCEP and showed little to no improvement with TCEP concentration increase; however, MgCl₂/PLURONIC F127 concentration increase resulted in significant assay performance improvement casting question on why the previous testing with 2M MgCl₂ resulted in poor performance with selected samples. This result also furthered the concern that not only is sample variability between donors playing a large part with regards to LFA performance but that the age of the individual sample is playing a considerable part as well. Specifically, the current experiment was designed to determine if improvement in sample variability issues for in-house samples could be improved via inclusion of TCEP or increase in sample buffer concentration. Initial testing show improvement with regards to sample 14 aggregation at the conjugate pad/membrane interface however this did not necessarily show improvement with regards to flow. Increase in solids and biotinylated antibody showed significant aggregation and poor flow issues not seen previously with other samples tested with the increased amounts, once again supporting that sample to sample differences are significant. Increase in salt and pluronic concentrations showed dramatic improvement with regards to line formation, flow, and aggregation reduction across the entire panel of samples. This improvement when concentration of MgCl₂ reached the 2M mark in the sample buffer did not present itself when previously tested using sample from the same donor suggesting that time of collection and/or time of storage may play a part in sample performance as well.

Lastly, a fourth panel of samples were tested. Initial results show little to no correlation with assigned values; however, further testing and observation appear to provide a pathway towards improving this correlation by reducing believed NSB and increasing specific binding. Specifically, the current experiment was designed to test nine new samples. Initial results show little to no correlation with the value assignment method as the highest and clinically diagnosed sample does not show higher test line intensity than the rest of the panel. The majority of samples show little to no test line formation which may be due to the low concentration of sFlt-1 assigned to those samples. Samples P0008 and P0013 generated significant test lines, much stronger than expected given their assigned values. In order to explore this, sample P0013 was retested with the addition of goat IgG and showed dramatic decrease in test line intensity similar to in-house samples 4, 12, and 15 (same donor). This reduction in assumed NSB may also apply to P0008; however, insufficient sample volume did not allow for further testing of this sample. One further observation that occurred during testing was the continued development of test lines over time most notably with sample P0012 which was assigned the highest value. This result may suggest that overall reduction in sensitivity compared to saliva spiked with recombinant or plasma native sFlt-1 is a result of lower binding affinity/rate and/or form of saliva native sFlt-1. Because of this, a longer run time may yield improved results.

### Example 17

Development work continued by first testing Goat IgG integration into the dried down LFA system. Results from testing showed that if Goat IgG and TCEP are to be included in the same LFA strip that they must be separated: with TCEP being included in the sample pad and Goat IgG included in the conjugate pad. Further examination of this format concluded that a minimum concentration of Goat IgG must be achieved in order for full effectiveness and that this minimum amount increases as the concentration of TCEP increases. Overall results do show differentiation between negative samples (S14, S15, S16, S17, S18, and S19) and positive sample (P0012) but the degree of differentiation appears variable from sample to sample and strip to strip.

Specifically, the current experiment was designed to test the effect of drying down Goat IgG into the strip, the location of the Goat IgG, and the concentration of TCEP that can work best with the Goat IgG. Results vary depending on the sample with regards to the impact of these components of the test result; however, the results from sample 15 provide strong evidence that goat IgG does not provide a blocking effect when combined with TCEP in the sample pad block buffer. Results also show that selected TCEP concentrations provided only mild differences between one another and that increasing the concentration to 1 mM does not negatively impact assay performance; this suggests that a higher concentration of TCEP may be possible. Time of development once again showed a general trend of increased test line intensity however this increase is variable depending on sample and strip components. Overall separation and differentiation between negative samples (S14 S15 S16 S19) and the positive sample (P0012) is achieved with the TCEP and Goat IgG dried into the LFA.

Another experiment was designed to further explore increased TCEP concentration with two Goat IgG locations: in the latex diluent or in both latex and biotinylated antibody diluents. Results again vary depending on sample but overall the difference between 1.5 and 2 mM TCEP appears negligible; however, based on previous results that tested P0012 with lower TCEP the aggregation does appear to be removed which may explain why full development appears to occur at 15 minutes while previously more time was required to achieve maximum test line development. A second result of note comes from the testing of sample 15 which has reacted the strongest to Goat IgG inclusion for in house samples. Results suggest that effectiveness of the IgG appears slightly reduced when the concentration of TCEP reaches 2 mM: this decrease in effectiveness may be recoverable by simply increasing the concentration of IgG in the system. Overall if considering the 15-minute time point, differentiation between negative samples (S14 S15 S17 and S18) and the positive sample (P0012) is achieved.

Previous experiments showed that as TCEP concentration increased, the blocking effect of Goat IgG was reduced. A further experiment was designed to determine if this reduction in the blocking effect of Goat IgG could be remedied by simply increasing the amount of Goat IgG in the system. Results do support this hypothesis by showing improved blocking resulting in reduction in TL intensity on average by ˜1 visual grade with sample 15 compared with previous results. Unfortunately, little to no performance increase is observed when TCEP concentration in increased from 1.5 to 2 mM in fact the previously observed reduction in aggregation with sample P0012 as the TCEP is increased was also not observed during this experiment. Differentiation with the current results can be made between negative samples S15 and S16 with positive sample P0012 however the difference between S14 and P0012 is negligible.

### Example 18

Development work continued by performing a conjugate pad screen using in house saliva samples to test overall performance. Initial testing included 4 different conjugate pads: Ahlstrom grade 8980 (glass), Ahlstrom grade 8951 (glass), GE Fusion 5 (proprietary), and Porex 4588 (Polymer based). Results from initial testing provided information with regards to flow speed and conjugate flow uniformity and by doing so narrowed the screen down to two possible candidates for replacement of the currently used Ahlstrom grade 6614 pad: Ahlstrom grade 8980 and Porex 4588.

Further testing with the two selected pads from initial testing against the currently used 6614 pad directly showed a significant difference in test and control line intensity and uniformity. The overall trend of test line intensity between 8980 and 6614 with regards to the three selected samples remained the same although the 8980 pad was producing significantly stronger lines; therefore, these two pads were tested side by side with lower concentrations of polystreptavidin in combination with the 8980 pad. Results suggest that a concentration of 0.25 mg/mL polystreptavidin in combination with the 8980 conjugate pad provide near equivalent results with regards to test line intensity with the currently used 1 mg/mL polystreptavidin in combination with the 6614 conjugate pad.

Specifically, an experiment was designed to perform a conjugate pad material screen. This development path was initiated due to uneven flow of conjugate up the membrane observed during testing with the currently used Ahlstrom grade 6614 pad which is likely leading to uneven test and control line formation. Results shown as images demonstrate differences in background and aggregation however flow dynamics have been presented in the provided table above. Overall Ahlstrom grade 8980 appears to provide the most favorable performance balancing flow speed with uniform line formation and lack of flooding when the full 110 μL of sample was added. The Porex pad provided the best release and flow however sample volume will likely need to be lowered as retention and metering of the sample by the pad is little to none. GE fusion 5 provided the overall worst performance although this may not be apparent in the presented results: this is due to its uneven and variable flow performance which includes uneven conjugate flow as well as variable flow speed and conjugate release. Ahlstrom 8951 performed acceptably once the conjugate had fully released however flow was consistently the slowest and likely saliva volume would need to be increased to achieve more optimal performance with this conjugate pad.

Another experiment was designed to continue conjugate pad material testing with pads that have been sprayed with latex conjugate and biotinylated antibody. It should be noted that control line and test line locations were flipped and although this was not intentional it does generate some interesting questions. Even with the flip of test and control line positions, generally expected trends of test line intensity were observed with samples S20 (same donor as S9, S14) generating a test line in all cases, S21 (same donor as S4, S15) generating the highest test line in all cases, and S22 (pooled) generating the overall lowest test line. Comparison of pads with regards to intensity of control and test lines is significant and the origin of this difference is currently unknown; however, it is known that Ahlstrom grade 8980 and Ahlstrom grade 6614 have ‘binders’ in the material as described by the manufacturer which may be playing a part in the chemistry of the assay and that pore size of each material is significantly different with the 6614 pad having the smallest pores and the Porex pad having the largest.

Yet another experiment was designed to examine if the test line intensity increase observed when using the 8980 conjugate pad could be controlled by reducing polystreptavidin concentration. Results do show a clear trend of test line intensity reduction as the concentration of polystreptavidin is reduced. Based on these results it appears that a polystreptavidin concentration of approximately 0.25 mg/mL in combination with the 8980 conjugate pad is most comparable to the currently used 1 mg/mL polystreptavidin concentration in combination with the 6614 conjugate pad. Example 19.

Development work continued by focusing on characterized sample testing. Initial testing showed significant differences between selected conjugate pads with regards to conjugate flow and test line formation/intensity. Results from initial testing with the two pads also contradicted previous testing by suggesting that the addition of goat IgG may provide an advantage in some cases but hurt others.

Based on the variable results, testing moved forward with the previously selected 6614 pad. Results from testing older samples were acceptable in that the result was consistent and no unexpectedly high signals appeared. A third panel of samples (the 6th in total) was then tested. Results from this third panel show little to no correlation with the value assignment method suggesting once again that while it is very likely the assay is capturing sFlt-1 in the unaltered saliva it is also capturing other elements as well as suffering from flow variation leading to variable interaction time between sandwiched conjugate and the test and control lines.

Specifically, an experiment was designed to test six new samples while simultaneously testing how conjugate pad effects the performance of the selected samples. Initial results show little to no correlation with the value assignment method although the overall values of the new samples do not provide a significant range and therefore the desired separation generated would have to come from a 1 ng/mL difference. Sample 22 did show significant NSB (even more so with the 8980 pad); however, this could be an artifact of the difference in polystreptavidin concentration resulting in a strong front edge binding for the 6614 pad (1 mg/mL polystreptavidin) and a more spread out binding with the 8980 pad (0.25 mg/mL polystreptavidin). Interestingly, this NSB was removed by taking the goat IgG out of the diluents in combination with the 8980 conjugate pad suggesting for the first time that goat IgG may hurt some samples by generating more NSB and help others by blocking it. Running sample P012 once again does show response which correlates to its high value, 6.2 ng/mL. The line intensity exhibited by this sample appears to rely to some extent on polystreptavidin concentration as the 8980 pad produces a significantly lower test line grade than the 6614. Interestingly in this case, removal of goat IgG appears to increase line intensity for the 6614 pad but decrease it for the 8980 pad.

Another experiment was designed to test a number of older samples using the current LFA in the current format not previously used when the samples were originally tested. While the test line does not appear to correlate to the assigned value, given the range of samples the results do not deviate dramatically from the assigned values as no sample generated a determinable amount of NSB as previously observed with certain samples. A second observation to note is that the slowest flowing samples, P0004 and P0032, generated the highest test line even though they were not assigned the highest values. This may point to a need for more interaction time with the test line to generate greater response with the faster flowing saliva samples or perhaps a further look into normalizing sample flow in order to reduce interaction with the test line for slower flowing samples.

Yet another experiment was designed to test eight new samples. Results show little to no correlation with the value assignment method. The majority of samples show test line formation which is likely a combination of sample variability along with the more active polystreptavidin lot even after 10% reduction in concentration at the test line. Sample P0041 did not flow to the wick even after an additional 20 of sample was added followed by 30 μL of chase buffer; suggesting that the system itself was clogged up to the point that fluid could no longer flow.

### Example 20. Further Characterization of LFA by Visual Grade and Automated Platform

We performed further characterization of our lateral flow device (LFA) that measures plasma sFLT1 and correlated with measurement of plasma sFLT1 on automated platform. Lateral flow device sFLT1 was measured by both visual grade and using Axxin Densitometry.

The test procedure included:

1) Pipette 15 μL of plasma sample into a borosilicate glass tube.
2) Press down the wick pad, the conjugate pad (also called “development region”), and sample pads, avoiding the membrane. This is vital to ensure that the strip runs properly, see FIG. 23A and FIG. 1A. For example, this includes pressing down on the overlap between the pick pad and the conjugate pad, pressing down on the overlap between the conjugate pad and the underlying membrane, and pressing down on the overlap between wick pad and the underlying membrane, avoiding directly contacting the membrane.
3) Drop the test strip into the glass tub so that the sample pad is in direct contact with the plasma sample.
4) Wait 30 seconds to allow sample to absorb into the strip.
5) Pipette 100 μL of chase solution into the bottom of each tube, being careful not to touch the strip with the end of the tip.
6) Allow strip to run for 14.5 additional minutes; see FIG. 23B.
7) Read the result by assigning a visual grad based on the DCN Visual Grade Chart (FIG. 11).
8) Remove the sample and conjugate pads.
9) Immediately read the strip with the Axxin (when using the Axxin, samples were spaced out by at least 30 seconds/strip to compensate for the amount of time the reader takes to read each strip).

Operation of Axxin is as follows:

a. Factory Option→Test Setup: Timer=On, Rapid Test=On, Test ID (auto number)=On.
b. Home screen: select “TEST” to begin testing strips→select “Test” option without Timer→select test type “1348” →Enter desired Test ID in the prompted screen→press the check mark icon.
c. The user may now insert the test into the reader and close the drawer. The strip should be inserted into the black holder wick pad first and pushed flush to the end. The strip should be centered and held in place with tape. The alignment of the black holder in the drawer will have the final orientation of the strip with the wick pad facing the operator. Once the drawer is closed, the reading will begin automatically. (FIG. 23C)
d. Once the reading is complete, a “Result” screen will be displayed. To ensure the test strip ran and was read properly, the screen should show “Valid” next to the word “Control”. The diagnosis of the test strip (positive/negative) will be displayed next to the “Result” Icon. If the user wishes to view the results, click on the Result button.
e. Results will be displayed as Area, Peak and Ratio for control and test line values. An image of the actual test strip will also be displayed.
f. To read the next strip, select the “test strip” icon on the bottom right of the screen.

Data can be exported to the Kinetic Viewer Software, which can export both the images of the test strip as well as data set. The AnalysisData data sheet will be organized in the order in which the test strips were run. The pertinent data to pay attention to are the Peak and Area columns for each strip. Reading from left to right, the first set of Peak and Area columns will be for the control line. The second set are for the test line. Typically the “Peak” values are used to quantify each line being analyzed, e.g., to calculate Control/Test peak data, or Test/Control peak data.

TABLE 13
Readings in Axxin and via visual grade of 11 samples.

| Sample ID | Sflt-1 | LFA Axxin | LFA Visual |
|---|---|---|---|
| 1 | 22576 | 2320 | 6.5 |
| 2 | 26904 | 2980 | 6.5 |
| 3 | 12603 | 1370 | 4.5 |
| 4 | 10591 | 1590 | 4.5 |
| 5 | 4024 | 450 | 0.5 |
| 6 | 4475 | 610 | 0.5 |
| 7 | 25422 | 2150 | 6.5 |
| 8 | 16307 | 1670 | 5.5 |
| 9 | 8782 | 760 | 2.5 |
| 10 | 6963 | 700 | 1.5 |
| 11 | 1837 | 320 | 0.5 |

These results above and FIG. 21A, 21B shows that LFA-Axxin densitometry through its automated measurement correlated with the sFLT1 significantly better than that noted with LFA-visual grade.

### Example 21. Evaluation of sFLT1 Measurement by LFA-Axxin

We then performed inter-assay coefficient of variations (CV) in 3 well-defined plasma samples (close to the limit of detection) to evaluate precision of sFLT1 measured by LFA-Axxin on six independent strips.

a. Sample A (LFA-Axxin value of 730)=Interassay CV was 13.2%
b. Sample B (LFA-Axxin value of 325)=interassay CV was 11.3%
c. Sample C (LFA-Axxin value of 256)=interassay CV was 14.6%

Summary: These results show that the LFA-Axinn device has CVs that are <15% which would be in the acceptable range for clinical use of this product.

### Example 22. Evaluation of Clinical Utility of LFA Device with 190 Patients' Samples

To evaluate clinical utility of LFA device, we studied 190 patients who presented with a suspicion of preeclampsia (PE) in the outpatient setting. Frozen serum samples were thawed; and sFLT1 levels were measured using the LFA-Axxin device and correlated with the development of preeclampsia with severe features (PE) over a 2-week period.

Among pregnant women presenting in the third trimester with suspicion of preeclampsia, sFLT1 measured using LFA-Axxin method had 97% negative predictive value (NPV) to rule out severe preeclampsia occurring over the subsequent 2 weeks. Positive predictive value (PPV), Sensitivity (SENS) and specificity (SPEC) are included in FIG. 22. Current standard of care is to admit patients with suspicion of severe disease. However as shown FIG. 22, of the 169 patients with an G1 value <1000, 49 were admitted, and we could have prevented 44 unnecessary admissions using the LFA-Axxin test. These data provide utility of this LFA device in the clinical situation to rule out preeclampsia with severe features in women with suspicion of preeclampsia.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

As used herein the term “comprising” or “comprises” is used in reference to compositions, methods, and respective component(s) thereof, that are useful to an embodiment, yet open to the inclusion of unspecified elements, whether useful or not. Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

1. A lateral flow device for detection of an analyte comprising one or more circulating fms-like tyrosine kinase 1 (Flt-1) protein isoforms in a biological sample, wherein the lateral flow device comprises:
a substrate comprising
a sample receiving region,
a development region,
an indication region, and
a quality control region,
wherein the substrate comprises a porous material and each region are in capillary contact with at least one other region thereby permitting a sample fluid to wick from the sample receiving region to the indication region;
a first capture reagent or a modification capable of immobilizing the first capture reagent at a first location of the indication region, wherein the first capture reagent is capable of forming a complex binding a first epitope of the Flt-1; and
a first detection reagent comprising a detectable label attached to an anti-Flt-1 antibody, wherein the first detection reagent is capable of forming a complex binding a second epitope of the Flt-1 and being transported from the development region to the indication region.

2. The lateral flow device of claim 1, wherein the indication region further comprises a second capture reagent or a modification capable of immobilizing the first capture reagent at a second location of the indication region.

3. The lateral flow device of claim 1, further comprising a second capture reagent comprising an antibody capable of binding the first detection reagent, the first detection reagent is capable of being transported from the development region to the quality control region, and the second capture reagent binds to a non-epitope binding domain of the first capture reagent but is not cross-reactive with the analyte.

4. The lateral flow device of claim 1, wherein the first detection reagent, the analyte, and the first capture reagent form a complex and give off a signal to indicate the presence and/or level of the analyte in the biological sample based on the presence and/or intensity of the detectable label.

5. The lateral flow device of claim 2, wherein the first detection reagent, the analyte, and the first capture reagent form a first complex and give off a first signal at the first location in the indication region, and the first detection reagent and the second capture reagent form a second complex and give off a second signal at the quality control region, and wherein the presence of both the first signal at the first location in the indication region and the second signal at the quality control indicates the presence of the analyte in the sample.

6. The lateral flow device of claim 1, wherein the sample receiving region when contacted with the biological sample takes up the biological sample and permits release of the biological sample towards the indication region.

7. The lateral flow device of claim 1, wherein the biological sample is obtained from an oral mucosa, and wherein the sample receiving region when contacted with the oral mucosa takes up oral fluid and is saturated with the oral fluid to permit release of the oral fluid towards the indication region.

8. The lateral flow device of claim 1, further comprising an end flow region comprising a porous material which conducts flow of the biological sample in the lateral flow device.

9. The lateral flow device of claim 1, wherein the analyte is all isoforms of soluble Flt-1, membrane-bound Flt-1, or a combination of both; and the first mobile detection reagent is a monoclonal or polyclonal antibody immunoreactive with the Flt-1.

10. The lateral flow device of claim 1, wherein the first detectable label is selected from a group consisting of a colloidal metal, colored particles, a liposome filled with a colored substance, an enzyme, a radiolabel, a chromophore and a fluorophore.

11. The lateral flow device of claim 1, wherein the sample receiving region comprises an Ahlstrom Grade 1281 pad, the development region comprises an Ahlstrom Grad 6614 pad, the lateral flow device comprises a nitrocellulose CN95 membrane as part of the indication region, the detectable label of the first detection reagent comprises colored latex bead, and the end flow region if present comprises an Ahlstrom Grade 243 pad.

12. The lateral flow device of claim 2, further comprising a housing having a cavity and an inspection site on the housing, wherein the indication region extends into the cavity along the housing to the inspection site to enable visual inspection of the first location and/or the second location of the indication region.

13. The lateral flow device of claim 1, wherein the sample receiving region comprises an Ahlstrom Grade 1281 pad, and the Ahlstrom Grade 1281 pad contains a detergent and magnesium chloride to reduce nonspecific binding; optionally the Ahlstrom Grade 1281 pad is presaturated with 0.5% Pluronic F127 detergent and 1M magnesium chloride as matrix reagents to block nonspecific binding within the development region and/or the indication region.

14. The lateral flow device of claim 1, wherein the indicator region comprises an Ahlstrom Grade 1281 pad, and the Ahlstrom Grade 1281 pad contains a detergent and magnesium chloride to reduce nonspecific binding; optionally the Ahlstrom Grade 1281 pad is presaturated with 0.5% Pluronic F127 detergent and 1M magnesium chloride as matrix reagents to block nonspecific binding within the development region and/or the indication region.

15. The lateral flow device of claim 1, wherein the sample receiving region, the development region and the indication region are in the form of a strip positioned above a base.

16. The lateral flow device of claim 1, configured for collecting the biological sample, wherein the biological sample is plasma, serum, whole blood, saliva, and/or urine from a pregnant woman.

17. A method of assaying a biological sample, or detecting a level, or a presence or absence, of an analyte in the biological sample, the analyte comprising soluble fms-like tyrosine kinase 1 (sFlt-1), bound Flt-1, or both, with a lateral flow device of claim 1, the method comprising:
applying the biological sample to the sample receiving region of the lateral flow device, so as to permit the biological sample to flow to the indication region, and
detecting the level, or the presence or absence, of the first detectable label at the first location in the indication region,
wherein, in the presence of the analyte, the first detection reagent, the analyte, and the first capture reagent form a complex.

18. The method of claim 17,
wherein the first detectable label is at a detectable level within 15 minutes from the application of the biological sample, or
wherein the biological sample is plasma, serum or whole blood from a pregnant woman; or wherein sFlt-1 or bound Flt-1 comprises an isoform encoded by mRNA of sFlt il3-short, sFlt1-il3-long, sFlt1-il4, sFlt1-e15a, sFlt1-e15b, or mFlt-1, or
wherein the analyte comprises sFlt-1, and the biological sample is obtained from a pregnant woman 18 weeks or later of pregnancy or until 20 weeks postpartum, or any combination thereof.

19. (canceled)

20. (canceled)

21. The method of claim 17, further comprising selecting a pregnant woman 18 weeks or later of pregnancy, before applying the biological sample obtained from the pregnant woman to the lateral flow device, wherein the analyte comprises sFlt-1.

22. A method of assaying a biological fluid sample of a pregnant woman, or determining the presence or absence of circulating fms-like tyrosine kinase 1 (Flt-1) protein isoforms in the biological fluid sample, with a lateral flow device of any of claims 2 16 claim 2, the method comprising:
applying the biological fluid sample to the sample receiving region of the lateral flow device, whose first immobile capture reagent is a monoclonal or polyclonal antibody only immunoreactive to Flt-1, so as to permit the biological fluid sample to flow to the indication region, and
detecting the presence or absence of the detectable label of the first detection reagent at the first location in the indication region,
wherein, in the presence of Flt-1, the first detection reagent, the analyte, and the first capture reagent form a complex that is deposited at the first location in the indication region, and
wherein the first detection reagent and the second capture reagent form another complex that is deposited at the quality control region.

23. The method of claim 22, further comprising detecting an amount of the detectable label of the first detection reagent at the first location in the indication region to indicate a quantity of the Flt-1 protein fragments in the biological fluid sample.

24. The method of claim 22, wherein the biological fluid sample comprises plasma, serum or whole blood from a subject.

25. A method of manufacturing a lateral flow device for detecting circulating fms-like tyrosine kinase 1 (Flt-1) protein fragments, the method comprising the steps of:
providing a base and providing a substrate positioned above the base, the substrate defining:
a sample receiving region,
an indication region, and
optionally a development region positioned between the sample receiving region and the indication region, or said optional development region overlapping with the sample receiving region and/or overlapping with the indication region,
each region comprises a porous material and is in capillary contact with at least one other region, thereby permitting a fluid to wick from the sample receiving region to the indication region;
immobilizing a first capture reagent or a modification capable of binding the first capture reagent at a first location in the indication region, wherein the first capture reagent comprises a monoclonal or polyclonal antibody specifically immunoreactive with Flt-1, or an antigen-binding fragment thereof;
providing a first detection reagent comprising a detectable label and an antibody or fragment thereof capable of binding the Flt-1, wherein the first detection reagent is capable of being transported from the development region to the indication region,
whereby the sample receiving region is configured to receive a fluid sample containing one or more analytes and to permit the fluid sample to wick to the indication region, and when the analytes comprise Flt-1, a complex is formed comprising the first detection reagent, the Flt-1, and the first capture reagent, and the complex indicates the presence of the Flt-1 through the detectable label at the first location in the indication region,
and optionally, further comprising:
providing at a second location in the indication region a second capture reagent or a modification capable of immobilizing the second capture reagent,
providing a second detection reagent capable of being transported to the indication region, the second detection reagent is capable of binding a house-keeping molecule in the fluid sample, and the second detection reagent, the house-keeping molecule and the second capture reagent form a complex to indicate a presence of the house-keeping molecule through the second detection reagent, wherein the second mobile detection reagent is not cross-reactive with Flt-1, with the first detection reagent or with the first capture reagent, and
optionally further providing an end flow region comprising a porous material and positioned such that a fluid is conducted from the sample receiving region through the indication region.

26. (canceled)

27. A method of determining a predisposition to preeclampsia or its related disorder, diagnosing preeclampsia or its related disorder, determining the likelihood of recurrence of preeclampsia or its related disorder, providing a prognosis for a subject with preeclampsia or its related disorder in a subject, or selecting a subject with preeclampsia or its related disorder for treatment with a therapy, comprising:
contacting a sample obtained from the subject with the sample receiving region of the lateral flow device of claim 5, and
detecting an amount of fms-like tyrosine kinase 1 (Flt-1) in the sample above a reference level,
wherein the sample obtained from the subject comprises a salivary sample, serum, plasma, whole blood, or urine.

28. The method of claim 27,
wherein the preeclampsia or its related disorder is preeclampsia or eclampsia with severe features, wherein the severe features comprises severe hypertension, or hypertension with one or more of thrombocytopenia, renal insufficiency, cerebral or visual symptoms, impaired liver function, and pulmonary edema, or
wherein the preeclampsia or its related disorder is an adverse outcome related to preeclampsia, wherein the adverse outcome related to preeclampsia comprises elevated liver function test, low platelet count, placental abruption, pulmonary edema, cerebral hemorrhage, convulsion, acute renal insufficiency, or maternal death, or
wherein the preeclampsia-related disorder comprises eclampsia, idiopathic fetal growth restriction, or hemolysis, elevated liver enzymes, low platelet count (HELLP) syndrome, or
both.

29. (canceled)

30. The method of claim 27, wherein the reference level is an amount of sFlt-1 from a sample of a non-pregnant woman or a woman not having preeclampsia, or an average amount of sFlt-1 from samples of a group of non-pregnant women or women not having preeclampsia.

31. (canceled)

32. The method of claim 27, further comprising digitally measuring an amount of the first signal and the amount of the second signal, wherein the digital measurement includes operating an application on a mobile or portable device.

33. The method of claim 27, wherein the detection comprises operating a densitometry instrument to obtain the amount of the Flt-1 protein fragments.

34. A method of administering a therapy for treatment and/or management of preeclampsia or eclampsia to a patient in need thereof, or selecting a patient for the therapy, comprising:
detecting an amount of fms-like tyrosine kinase 1 (Flt-1) protein fragments with a lateral flow device of claim 1, and
administering an effective amount of a steroid, magnesium sulfate, an anti-Flt-1 antibody and/or therapeutic apheresis to lower the sFlt1 in the patient in need thereof for treating and/or reducing the progression of preeclampsia or eclampsia.
